(19) United States
(12) Patent Application Publication
(10) Pub. No.: US 2026/0242365 A1
(43) Pub. Date: Aug. 20, 2026

(54) USES OF CERTAIN 1,4-OXAZEPANE-2-CARBOXAMIDES AS DPP1 INHIBITORS

(71) Applicant: Insmed Incorporated, Bridgewater, NJ (US)

(72) Inventor: Adam J. PLAUNT, Bridgewater, NJ (US)

(73) Assignee: Insmed Incorporated, Bridgewater, NJ (US)

(21) Appl. No.: 19/156,134

(22) PCT Filed: Feb. 14, 2024
(86) PCT No.: PCT/US2024/015802

§ 371 Date: Aug. 13, 2025

Related U.S. Application Data
(60) Provisional application No. 63/445,955, filed on Feb. 15, 2023.
(60) Provisional application No. 63/599,868, filed on Nov. 16, 2023.

(51) Int. Cl. C07D 413/12; A61K 31/553; C07D 267/10; C07D 417/12

(52) U.S. Cl. CPC C07D 413/12; A61K 31/553; C07D 267/10; C07D 417/12

(57) ABSTRACT

Provided herein are compositions comprising an effective amount of a DPP1 inhibitor of Formula (I), or a pharmaceutically acceptable salt thereof, and their use in treating disorders mediated by dipeptidyl peptidase 1 (DPP1). In embodiments of the methods provided herein, the composition is administered orally, once-a-day.

### CROSS REFERENCE TO RELATED APPLICATIONS

This application claims priority to U.S. Provisional Application No. 63/445,955 filed Feb. 15, 2023, and U.S. Provisional Application No. 63/599,868, filed Nov. 16, 2023, the contents of which are hereby incorporated by reference in their entities for all purposes.

### BACKGROUND OF THE INVENTION

Dipeptidyl peptidase 1 (DPP1), also known as cathepsin C, is a type of lysosome cysteine protease capable of removing the dipeptide from the amino terminal of protein substrates. The earliest discovery of DPP1 was by Gutman and Fruton in 1948 (J Biol Chem, 174, 851-858); subsequently, its earliest description in human cDNA occurred in 1995 (FEBS Lett, 369, 326-330). DPP1 is the only member of the papain-like protease family that has a tetramer effect, and is composed of four identical subunits. Each subunit is composed of an N-terminal fragment, a heavy chain and a light chain (J Biol Chem, 270, 21626-21631).

High levels of DPP1 are expressed by many tissues of the lungs, kidneys, liver and spleen (Biol. Chem. Hoppe Seyler 373:367-373, 1992). As such it has the same role in activating serine protease in haematopoietic stem cells, and there is also relatively high expression of DPP1 in neutrophils, cytotoxic lymphocytes, natural killer cells, alveolar macrophages and mastocytes. The latest data has shown that, apart from being an important enzyme in lysosomal protein degradation, DPP1 also plays a role as a key enzyme in the activation of the following cell serine protease particles: cytotoxic T lymphocytes and natural killer cells (granzymes A and B; Proc. Nat. Acad. Sci. 96:8627-8632, 1999), mastocytes (chymotrypsin and fibrinogenase; J Biol. Chem. 276:18551-18556, 2001), and neutrophils (cathepsin G, elastinase and protease hydrolase 3; J Clin. Invest. 109:363. 371, 2002). Once activated, these proteases can cause degradation of multiple extracellular matrix components, resulting in tissue damage and chronic inflammation. Due to DPP1 playing a key role in the activation of these proteases, it has become considered to be a type of effective therapeutic target (J Clin Invest, 2002, 109, 363-271; J Immunol, 2004, 173, 7277-7281).

In light of this, cathepsin C inhibitors exhibit potential for use in treatment in various inflammatory diseases. In view of the effect of DPP1 on certain pro-inflammatory serine proteases, clinical applications that suppress its activity and therefore suppress downstream serine protease activity may have favorable prospects. Currently, there are a number of related patents that give reports of synthesis of DPP1 inhibitors, for example, WO2004/110988 relates to a nitrile derivative and its use as a DPP1 inhibitor. WO2009/074829 relates to peptidyl nitriles and their use as DPP1 inhibitors. WO2010/128324 relates to α-aminonitriles and their use as DPP1 inhibitors. WO2012/119941 relates to peptidyl nitriles and their use as DPP1 inhibitors. WO2013/041497 relates to N-[−1-cyano-2-(phenyl)ethyl)-2-azabicyclo[2.2.1]heptane-3-carboxamide and its use as a DPP1 inhibitor. WO2001/096285 and WO2003/048123 relate to β-aminonitriles and their cysteine protease inhibitory activity.

Because DPP1 has been implicated in a number of disease states, and because no DPP1 inhibitors have been approved by a regulatory authority, there remains a need for novel DPP1 inhibitors.

### SUMMARY OF THE INVENTION

In one aspect, the present invention provides a method of treating a disorder associated with altered DPP1 activity in a subject in need of treatment, comprising administering an effective amount of a compound of formulae (I), or a pharmaceutically acceptable salt thereof, to the subject for an administration period:

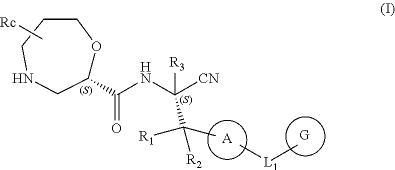

wherein,

G is a 5-12 membered carbon ring, a 5-12 membered monocyclic heterocycle containing 1-3 heteroatoms selected from N, S or O or the fused ring of formula (I-1)

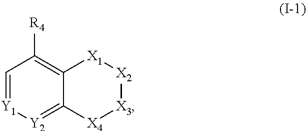

L₁ bonds G by replacing any hydrogen atom on ring G, and is a bond, C₁₋₃ alkylene, —NH—, —N(C₁₋₄alkyl)-, —O—, —S—, C₂₋₆ortho-alkenyl, C₂₋₆ortho-alkynyl, —CO— or —CONH—, wherein the alkylene, ortho-alkenyl or ortho-alkynyl is optionally substituted with 1-3 halogen, C₁₋₄ alkyl, cyano, hydroxyl, NH₂ and —COOH groups;

R₁, R₂ and R₃ are independently selected from H, deuterium, halogen, C₁₋₄alkyl, C₁₋₄alkoxy, C₂₋₆alkenyl, C₂₋₆alkenyl, C₂₋₆alkynyl, C₃₋₆cycloalkyl and a 4-7 membered heterocycle containing 1-3 heteroatoms selected from N, S or O, the alkyl, alkoxy, alkenyl, alkynyl, cycloalkyl and heterocycle having arbitrarily undergone substitution with 1-3 groups selected from halogen, C₁₋₄alkyl, cyano, hydroxyl, NH₂ and COOH; alternatively, R₁ and R₂ form a C₃₋₆cycloalkyl or a 4-7 membered heterocycle containing 1-3 heteroatoms selected from N, S or O, wherein the cycloalkyl or heterocycle is optionally substituted with 1-3 groups selected from ═O, halogen, cyano, hydroxyl, NH₂, COOH, C₁₋₄ alkyl, C₂₋₆ alkenyl, C₂₋₆alkynyl and C₃₋₆cycloalkyl;

Y₁ and Y₂ are both independently selected from CR₄ or N;

each R₄ is independently selected from H, deuterium, halogen, C₁₋₄ alkyl, C₁₋₄ alkoxy, C₂₋₆ alkenyl, C₂₋₆ alkynyl, C₃₋₆cycloalkyl, cyano, hydroxyl, NH₂, NHC₁₋₄ alkyl, N(C₁₋₄ alkyl) 2, COOH, COC₁₋₄ alkyl, COOC₁₋₄ alkyl, CONHC₁₋₄ alkyl, CON(C₁₋₄ alkyl) 2, NHCOC₁₋₄ alkyl, and a 4-7 membered heterocycle containing 1-3 heteroatoms selected from N, S or O; wherein the alkyl, alkenyl, alkynyl, cycloalkyl and heterocycle groups within R₄ are optionally substituted with 1-3 groups selected from deuterium, halogen, cyano, hydroxyl, NH₂ and COOH;

X₁, X₂, X₃ and X₄ are each independently selected from a bond, NR₅, O, CR₆R₇, S, S(O) and S(O) 2, and wherein at most one of X₁, X₂, X₃ and X₄ is a bond;

Rc is H, ═O, halogen, cyano, hydroxyl, NH₂, —COOH, C₁₋₄ alkyl, halogenated C₁₋₄ alkyl, hydroxyl C₁₋₄ alkyl, C₁₋₄ alkoxy, C₁₋₆ alkoxyalkyl or halogenated C₁₋₄ alkoxy;
each R₅ is independently selected from H, C₁₋₄ alkyl, —COC₁₋₄ alkyl, C₂₋₆ alkenyl, C₂₋₆ alkynyl and C₃₋₆cycloalkyl; wherein the alkyl, alkenyl, alkynyl and cycloalkyl is optionally substituted with 1-3 groups selected from deuterium, halogen, cyano, hydroxyl, NH₂ and COOH;
R₆ and R₇ are each independently selected from H, deuterium, halogen, C₁₋₄ alkyl, C₁₋₄alkoxy, C₂₋₆ alkenyl, C₂₋₆ alkynyl, C₃₋₆cycloalkyl, cyano, hydroxyl, NH₂, COOH, and a 4-7 membered heterocycle containing 1-3 heteroatoms selected from N, S or O, wherein the alkyl, alkoxy, alkenyl, alkynyl, cycloalkyl and heterocycle is optionally substituted with 1-3 groups arbitrarily selected from deuterium, halogen, cyano, hydroxyl, NH₂ and —COOH;
alternatively, R₆ and R₇ form ═O;
alternatively, two R₅ on the atoms in adjacent rings or two R₆ on the atoms in adjacent rings within X₁, X₂, X₃ and X₄, or the R₅ and R₆ of atoms on adjacent rings and the atoms binding to them form double-bonds;
alternatively, the R₆ and R₇ on the same carbon atom form a C₃₋₁₂ carbon ring with the carbon atoms linked to them or a 4-7 membered heterocycle containing 1-3 heteroatoms selected from N, S or O, said carbon ring or heterocycle having arbitrarily undergone substitution with 1-3 groups selected from ═O, halogen, cyano, hydroxyl, NH₂, —COOH, C₁₋₄ alkyl, C₂₋₆ alkenyl, C₂₋₆ alkynyl and C₃₋₆ cycloalkyl;
Z is CH or N;

A is

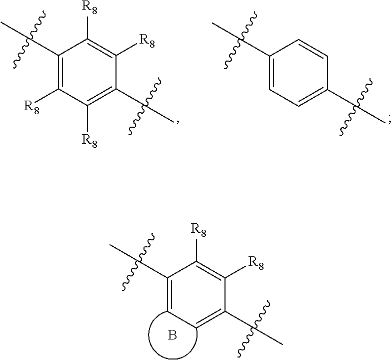

a hexatomic heteroaryl or 5-7 membered non-aromatic monocyclic heterocycle, wherein the heteroaryl and non-aromatic monocyclic heterocycle contains 1-3 heteroatoms selected from N, S or O; and wherein “*” represents the terminal binding with the alkyl carbon;
B is a C₄₋₆ carbon ring or a 5-6 membered heterocycle containing 1-3 heteroatoms selected from N, S or O;
each R₅ is independently selected from H, —O, deuterium, halogen, C₁₋₄ alkyl, C₁₋₄ alkoxy, SC₁₋₄ alkyl, C₂₋₆ alkenyl, C₂₋₆ alkynyl, cyano, hydroxyl, —COOH, NH₂ and C₃₋₆ cycloalkyl; said alkyl, alkoxy, alkenyl, alkynyl or cycloalkyl having arbitrarily undergone substitution with 1-3 groups selected from deuterium, halogen, cyano, hydroxyl, NH₂ and —COOH.

In one embodiment of a compound of Formula (I), or a pharmaceutically acceptable salt thereof, the compound is a compound of Formula (Ia), or a pharmaceutically acceptable salt thereof:

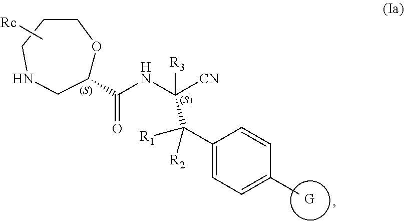

wherein,
R₁, R₂, R₃ and G are defined above for Formula (I); and
Rc is ═O, halogen, cyano, hydroxyl, NH₂, —COOH, C₁₋₄ alkyl, halogenated C₁₋₄ alkyl, hydroxyl C₁₋₄ alkyl, C₁₋₄ alkoxy, C₁₋₆ alkoxyalkyl or halogenated C₁₋₄ alkoxy.

In one embodiment of a compound of Formula (I), or a pharmaceutically acceptable salt thereof, the compound is a compound of Formula (Ib), or a pharmaceutically acceptable salt thereof:

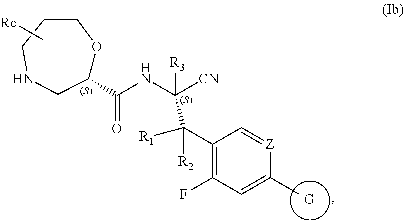

wherein,
R₁, R₂, R₃, Z and G are defined above for Formula (I); and
Rc is ═O, halogen, cyano, hydroxyl, NH₂, —COOH, C₁₋₄ alkyl, halogenated C₁₋₄ alkyl, hydroxyl C₁₋₄ alkyl, C₁₋₄ alkoxy, C₁₋₆ alkoxyalkyl or halogenated C₁₋₄ alkoxy.

In one embodiment, the compound for use in one of the methods provided herein, or a pharmaceutically acceptable salt thereof, is selected from one of the following:

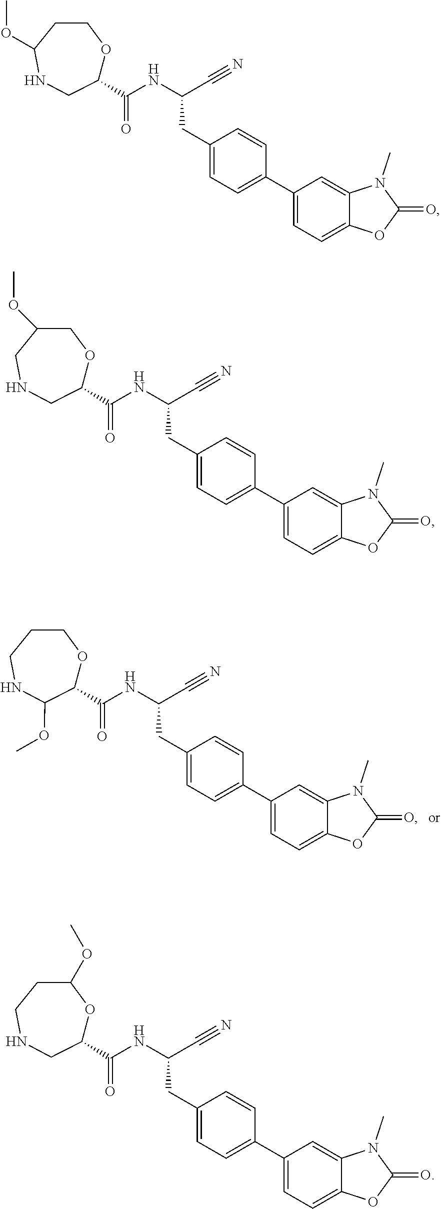 [SMILES: COC1CCO[C@H](C(=O)N[C@H](C#N)Cc2ccc(-c3ccc4oc(=O)n(C)c4c3)cc2)CN1 ; COC1CNC[C@@H](C(=O)N[C@H](C#N)Cc2ccc(-c3ccc4oc(=O)n(C)c4c3)cc2)OC1 ; COC1NCCCO[C@@H]1C(=O)N[C@H](C#N)Cc1ccc(-c2ccc3oc(=O)n(C)c3c2)cc1 ; COC1CCNC[C@@H](C(=O)N[C@H](C#N)Cc2ccc(-c3ccc4oc(=O)n(C)c4c3)cc2)O1]

In one embodiment, the compound for use in one of the methods provided herein, or a pharmaceutically acceptable salt thereof, is selected from one of the following:

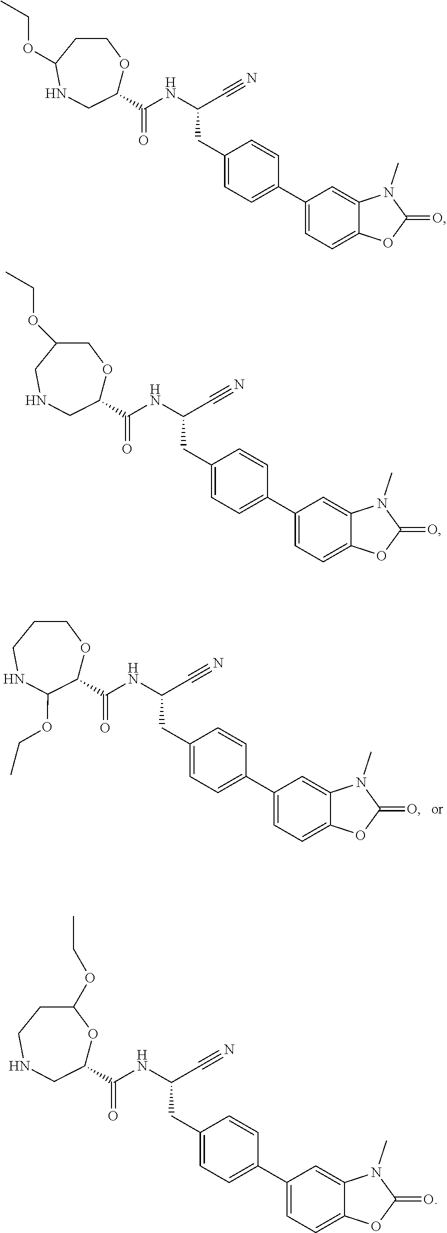 [SMILES: CCOC1CCO[C@H](C(=O)N[C@H](C#N)Cc2ccc(-c3ccc4oc(=O)n(C)c4c3)cc2)CN1 ; CCOC1CNC[C@@H](C(=O)N[C@H](C#N)Cc2ccc(-c3ccc4oc(=O)n(C)c4c3)cc2)OC1 ; CCOC1NCCCO[C@@H]1C(=O)N[C@H](C#N)Cc1ccc(-c2ccc3oc(=O)n(C)c3c2)cc1 ; CCOC1CCNC[C@@H](C(=O)N[C@H](C#N)Cc2ccc(-c3ccc4oc(=O)n(C)c4c3)cc2)O1]

In another embodiment, the compound for used in one of the methods provided herein,
or a pharmaceutically acceptable salt thereof, is

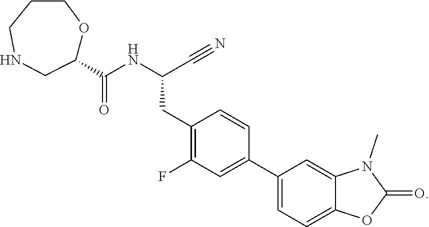 [SMILES: Cn1c(=O)oc2ccc(-c3ccc(C[C@@H](C#N)NC(=O)[C@@H]4CNCCCO4)c(F)c3)cc21]

In one embodiment, the compound for use in one of the methods provided herein, or a pharmaceutically acceptable salt thereof, is selected from one of the following:

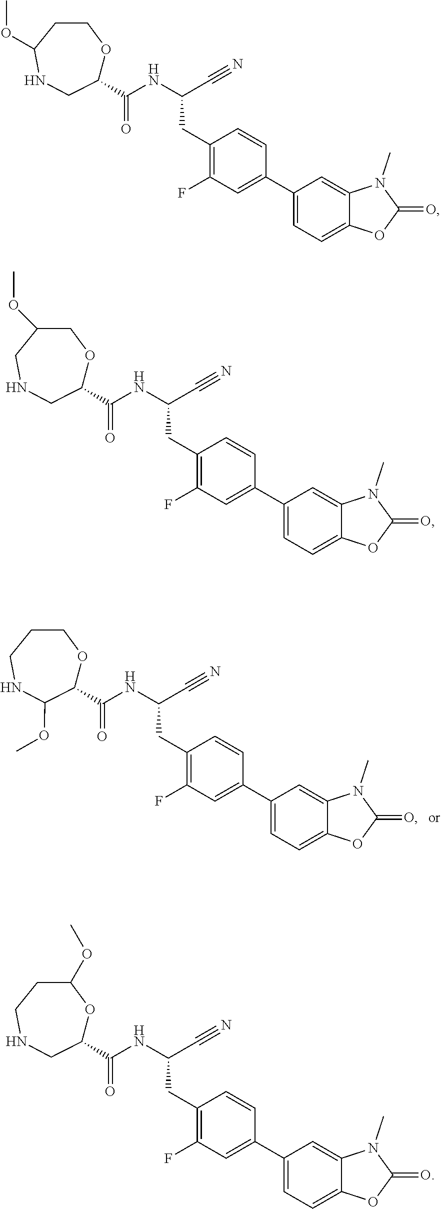 [SMILES: COC1CCO[C@H](C(=O)N[C@H](C#N)Cc2ccc(-c3ccc4oc(=O)n(C)c4c3)cc2F)CN1 ; COC1CNC[C@@H](C(=O)N[C@H](C#N)Cc2ccc(-c3ccc4oc(=O)n(C)c4c3)cc2F)OC1 ; COC1NCCCO[C@@H]1C(=O)N[C@H](C#N)Cc1ccc(-c2ccc3oc(=O)n(C)c3c2)cc1F ; COC1CCNC[C@@H](C(=O)N[C@H](C#N)Cc2ccc(-c3ccc4oc(=O)n(C)c4c3)cc2F)O1]

In one embodiment, the compound for use in one of the methods provided herein, or a pharmaceutically acceptable salt thereof, is selected from one of the following:

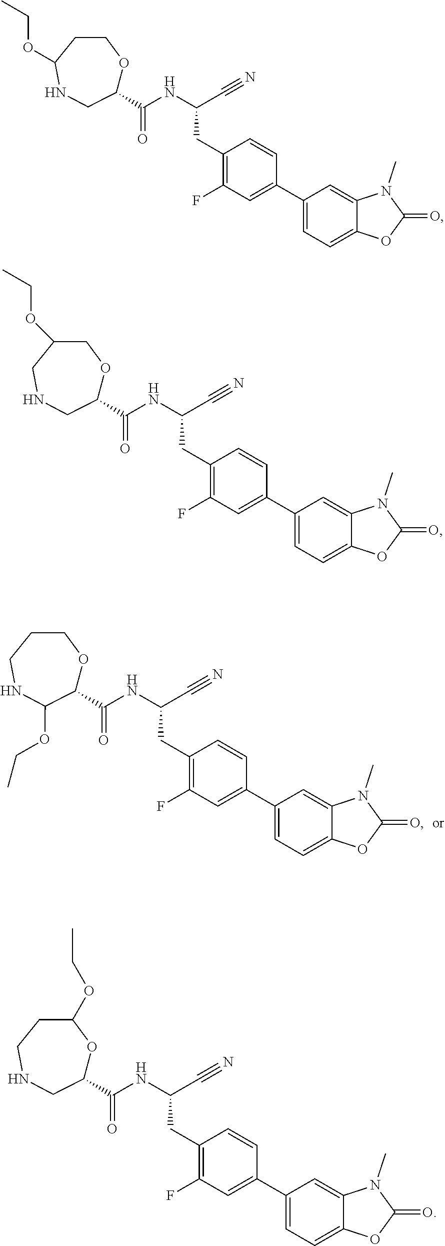 [SMILES: CCOC1CCO[C@H](C(=O)N[C@H](C#N)Cc2ccc(-c3ccc4oc(=O)n(C)c4c3)cc2F)CN1 ; CCOC1CNC[C@@H](C(=O)N[C@H](C#N)Cc2ccc(-c3ccc4oc(=O)n(C)c4c3)cc2F)OC1 ; CCOC1NCCCO[C@@H]1C(=O)N[C@H](C#N)Cc1ccc(-c2ccc3oc(=O)n(C)c3c2)cc1F ; CCOC1CCNC[C@@H](C(=O)N[C@H](C#N)Cc2ccc(-c3ccc4oc(=O)n(C)c4c3)cc2F)O1]

In yet another embodiment, the compound for use in one of the methods provided herein, or a pharmaceutically acceptable salt thereof, is selected from one of the following:

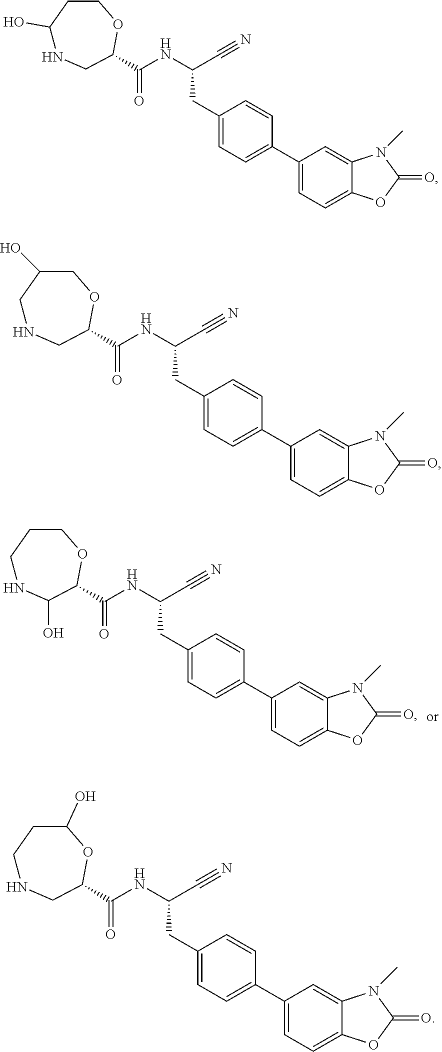 [SMILES: Cn1c(=O)oc2ccc(-c3ccc(C[C@@H](C#N)NC(=O)[C@@H]4CNC(O)CCO4)cc3)cc21 ; Cn1c(=O)oc2ccc(-c3ccc(C[C@@H](C#N)NC(=O)[C@@H]4CNCC(O)CO4)cc3)cc21 ; Cn1c(=O)oc2ccc(-c3ccc(C[C@@H](C#N)NC(=O)[C@H]4OCCCNC4O)cc3)cc21 ; Cn1c(=O)oc2ccc(-c3ccc(C[C@@H](C#N)NC(=O)[C@@H]4CNCCC(O)O4)cc3)cc21]

In yet even another embodiment, the compound for use in one of the methods provided herein, or a pharmaceutically acceptable salt thereof, is selected from one of the following:

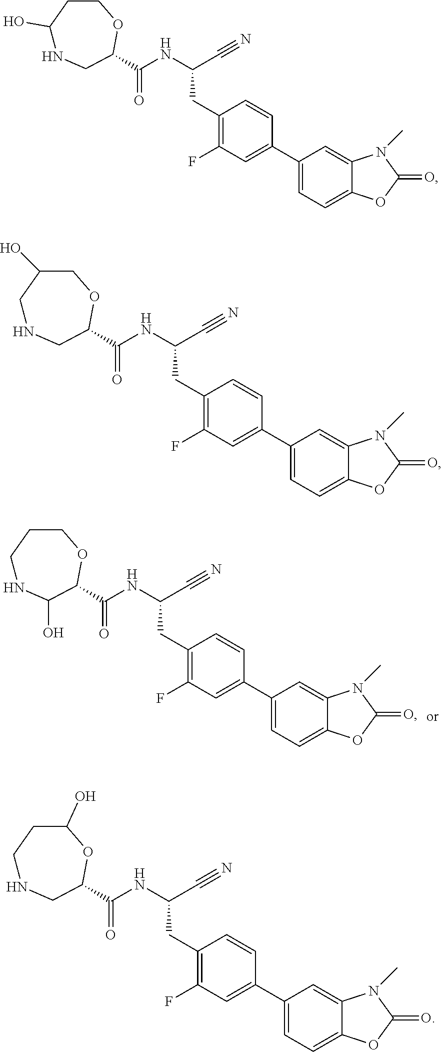 [SMILES: Cn1c(=O)oc2ccc(-c3ccc(C[C@@H](C#N)NC(=O)[C@@H]4CNC(O)CCO4)c(F)c3)cc21 ; Cn1c(=O)oc2ccc(-c3ccc(C[C@@H](C#N)NC(=O)[C@@H]4CNCC(O)CO4)c(F)c3)cc21 ; Cn1c(=O)oc2ccc(-c3ccc(C[C@@H](C#N)NC(=O)[C@H]4OCCCNC4O)c(F)c3)cc21 ; Cn1c(=O)oc2ccc(-c3ccc(C[C@@H](C#N)NC(=O)[C@@H]4CNCCC(O)O4)c(F)c3)cc21]

The compounds and methods provided herein can be used to treat any disease where altered DPP1 activity is thought to play a role. For example, in one embodiment, the method of treatment is a method of treating an obstructive disease of the airway, e.g., cystic fibrosis (CF), asthma or bronchiectasis (e.g., non-CF bronchiectasis).

In another embodiment, the method of treatment is a method for treating chronic rhinosinusitis (CRS). In some embodiments, the method of treatment is a method for treating hidradenitis suppurativa (HS). In some embodiments, the method of treatment is a method for treating cancer. In some embodiments, the method of treatment is a method of treating lupus nephritis. In some embodiments, the method of treatment is a method of treating rheumatoid arthritis. In some embodiments, the method of treatment is a method of treating inflammatory bowel disease (IBD). In some embodiments, the method of treatment is a method of treating Granulomatosis with polyangiitis (GPA).

In yet another embodiment, the method of treatment is a method for giant cell arteritis, polyarteritis nodosa, anti-GBM disease (Goodpasture's), systemic scleroderma, diabetic nephropathy, diabetic neuropathy, diabetic retinopathy, diabetic ulcers, Duchenne muscular dystrophy, bronchiolitis obliterans, atopic dermatitis, pyoderma gangrenosum, sweet's syndrome, dermatomyositis/polymyositis, neutrophilic dermatoses, thrombosis, bronchopulmonary dysplasia, amyotrophic lateral sclerosis, sickle cell anemia, psoriasis, or a ventilator-induced lung injury.

### BRIEF DESCRIPTION OF THE FIGURES

FIG. 1 is a graph of percent inhibition of human DPP1 as a function of INSM-201 concentration, with the IC50 shown at the dashed line.

FIG. 2 is a graph is a graph of percent inhibition of mouse DPP1 as a function of INSM-201 concentration, with the IC50 shown at the dashed line.

FIG. 3 is a graph of percent inhibition of DPP1 in HL-60 cells as a function of INSM-201 concentration, with the IC50 shown at the dashed line.

FIG. 4 is a graph of percent inhibition of human DPP1 as a function of INSM-202 concentration, with the IC50 shown at the dashed line.

FIG. 5 is a graph of percent inhibition of mouse DPP1 as a function of INSM-202 concentration, with the IC50 shown at the dashed line.

FIG. 6 is a graph of percent inhibition of DPP1 in HL-60 cells as a function of INSM-202 concentration, with the IC50 shown at the dashed line.

FIG. 7 is a graph of percent inhibition of human DPP1 as a function of INSM-202 concentration, with the IC50 shown at the dashed line.

FIG. 8 is a graph of percent inhibition of mouse DPP1 as a function of INSM-203 concentration, with the IC50 shown at the dashed line.

FIG. 9 is a graph of percent inhibition of DPP1 in HL-60 cells as a function of INSM-203 concentration, with the IC50 shown at the dashed line.

### DETAILED DESCRIPTION OF THE INVENTION

### Terminology

If not specifically described in the present invention, terms of the present invention have the following meanings:

The carbon, hydrogen, oxygen, sulphur, nitrogen or halogen in relation to the groups and compounds mentioned in the present invention all include their isotopes, and, the carbon, hydrogen, oxygen, sulphur, nitrogen or halogen in relation to the groups and compounds mentioned in the present invention more preferably are replaced by one or more of their corresponding isotopes, whereby the isotopes of carbon include ¹²C, ¹³C and ¹⁴C, the isotopes of hydrogen include protium (H), deuterium (deuterium is also referred to as deuterohydrogen), tritium (T, also referred to as heavy hydrogen), the isotopes of oxygen including ¹⁶O, ¹⁷O and ¹⁸O, the isotopes of sulphur including ³²S, ³³S, ³⁴S and ³⁶S, the isotopes of nitrogen including ¹⁴N and ¹⁵N, the isotope of fluorine ¹⁹F, the isotopes of chlorine including ³⁵Cl and ³⁷Cl, and the isotopes of bromine including ⁷⁹Br and ⁸¹Br.

“Halogen” in this document refers to F, Cl, Br, I or their isotopes.

“Halogenated” or “halogen substitution” refers to substitution by one or more of the above F, Cl, Br or I or their isotopes, the upper limit of the number of halogen substitution groups is equal to the total number of substitutable hydrogens, and unless specifically defined, the number of halogen substitution groups is any integer between 1 and that upper limit, if the number of halogen substitution groups is greater than 1, substitution by the same or a different halogen is possible. Customarily, 1-5 halogen substitutions, 1-3 halogen substitutions, 1-2 halogen substitution and 1 halogen substitution are involved.

“Deuterium” refers to an isotope of hydrogen (H).

“Deuteration” refers to the hydrogen atoms on an alkyl, cycloalkyl, alkylene, aryl, heteroaryl, alkenyl or alkynyl group undergoing deuteration by at least one isotope, the upper limit of deuteration is equal to the total number of substitutable hydrogens of the substituted groups, and unless specifically defined, the deuteration number is any integer between 1 and the upper limit, preferably being 1-20 deuterium atom substitutions, more preferably being 1-10 deuterium atom substitutions, yet more preferably being 1-6 deuterium atom substitutions, even more preferably being 1-3 deuterium atoms substitutions.

A “C_{x-y}” group refers to a group which contains×to y carbon atoms, for instance “C₁₋₆ alkyl” refers to an alkyl that contains 1-6 carbon atoms.

“Alkyl” refers to a monovalent straight chain or branch-chain saturated aliphatic hydrocarbon group, and if there is no special explanation, it is an alkyl with 1 to 20 carbon atoms, preferably being an alkyl with 1 to 8 carbon atoms, more preferably being an alkyl with 1 to 6 carbon atoms, and yet more preferably being an alkyl with 1 to 4 carbon atoms. Non-restrictive embodiments include methyl, ethyl, n-propyl, isopropyl, n-butyl, sec-butyl, neobutyl, tert-butyl, n-pentyl, isopentyl, neopentyl, n-hexyl and their various branch-chain isomers.

“Alkylene” refers to a divalent straight chain and branch-chain alkyl. Embodiments of alkylene include but are not limited to methylene and ethylidene etc.

“Halogenated alkyl” refers to the situation where one or more hydrogens in an alkyl are replaced by one or more halogen atoms (i.e. fluorine, bromine, iodine or their isotopes), the upper limit of the number of halogen substitution groups is equal to the total number of hydrogens substitutable in the alkyl, and unless specifically defined, the number of halogen substitution groups is any integer between 1 and the upper limit. Customarily, alkyls undergo 1-5 halogen substitutions, or 1-3 halogen substitutions, or 1-2 halogen substitutions or 1 halogen substitution; if the number of halogen substitution groups is greater than 1, substitution with the same or a different halogen can take place; specific examples include but are not limited to —CF₃, —CH₂Cl, —CH₂CF₃, —CCl₂ and CF₃ etc.

“Alkoxy” or “alkyloxy” refers to —O-alkyl. For instance-O—C₁₋₈ alkyl, —O—C₁₋₆ alkyl, —O—C₁₋₄ alkyl or —O—C₁₋₂ alkyl. Specific non-restrictive embodiments include methoxy, ethoxy, n-propoxy, isopropoxy, n-butoxy, sec-butoxy, tert-butoxy, n-pentyloxy, n-hexaoxy, cyclopropoxy and cyclobutoxy etc.; said alkoxy can be arbitrarily substituted by a substitution group.

“Halogenated alkoxy” refers to —O-halogenated alkyl. For instance-O-halogenated C₁₋₈ alkyl, —O-halogenated C₁₋₆ alkyl, —O-halogenated C₁₋₄ alkyl, or —O-halogenated C₁₋₂ alkyl; the upper limit of the halogen substitution groups is equal to the total number of substitutable hydrogens of the substituted group, and unless specifically defined, the number of halogen substitution groups is any integer between 1 and the upper limit, preferably being 1-5 halogen substitutions, 1-3 halogen substitutions, 1-2 halogen substitutions or 1 halogen substitution; if the number of halogen substitution groups is greater than 1, substitution by the same or a different halogen is possible; non-restrictive embodiments include monofluoromethoxy, difluoromethoxy, trifluoromethoxy and difluoroethoxy etc.

“Alkylamine” or “alkanamine” refers to an ammonia group that undergoes substitution by one or two alkyls, it is also written-N-(alkyl) 2 or—NH-alkyl, the latter also being written as monoalkylamine. Non-restrictive embodiments include dimethylamine, monomethylamine, diethylamine and monoethylamine etc.

“Alkenyl” refers to a straight chain hydroxyl or branch-chain hydroxyl which includes at least one carbon-carbon double bond (C═C), these customarily include 2 to 18 carbon atoms, for instance 2 to 8 carbon atoms, as well as 2 to 6 carbon atoms, and even 2 to 4 carbon atoms, examples include but are not limited to ethenyl, allyl, 1-propenyl, 2-propenyl, 1-butenyl, 2-butenyl, 3-butenyl, 1-pentenyl, 2-pentenyl, 3-pentenyl, 4-pentenyl, 1-methyl-1-butenyl, 2-methyl-1-butenyl, 2-methyl-3-butenyl, 1-hexenyl, 2-hexenyl, 3-hexenyl, 4-hexenyl, 5-hexenyl, 1-methyl-1-pentenyl, 2-methyl-1-pentenyl, 1-heptenyl, 2-heptenyl, 3-heptenyl, 4-heptenyl, 1-octenyl, 3-octenyl, 1-nonenyl, 3-nonenyl, 1-decenyl, 4-decenyl, 1,3-butadiene, 1,3-pentadiene, 1,4-pentadiene and 1,4-hexadiene etc.; said alkenyl may furthermore by arbitrarily substituted by any substitution group.

“Ortho-alkenyl” refers to a straight chain or branch-chain divalent unsaturated hydroxyl containing at least one carbon-carbon double bond (C═C), and unless specifically stated, ortho-alkynyl contains 2-6 carbon atoms, preferably containing 2-4 carbon atoms, unrestrictive embodiments including ortho-ethynyl, and the alkenyl group may undergo substitution arbitrarily with a substitution group.

“Alkynyl” is a straight chain hydroxyl or branch-chain hydroxyl containing at least one carbon-carbon triple-bond (C≡C), customarily consisting of 2 to 18 carbon atoms, preferably consisting of 2 to 8 carbon atoms, preferably consisting of 2 to 6 carbon atoms, and even 2 to 4 carbon atoms, examples include but are not limited to ethynyl, 1-propinyl, 2-propinyl, butynyl, 2-butynyl, 3-butynyl, 1-methyl-2-propinyl, 4-pentynyl, 3-pentynyl, 1-methyl-2-butynyl, 2-hexynyl, 3-hexynyl, 2-heptynl, 3-heptynl, 4-heptynl, 3-octynyl, 3-nonynyl and 4-decynyl etc.; said alkynyl may undergo substitution arbitrarily with a substitution group.

“Ortho-alkenyl” refers to a straight chain or branch-chain divalent unsaturated hydroxyl containing at least one carbon-carbon double bond (C═C), customarily containing 2-6 carbon atoms, preferably containing 2-4 carbon atoms, unrestrictive embodiments including ortho-ethynyl, and the alkenyl group may undergo substitution arbitrarily with a substitution group. “Cycloalkyl” refers to a saturated or partially unsaturated, non-aromatic carbocyclic hydroxyl containing no heterocyclic atoms. Cycloalkyls can be single rings, double rings or multiple rings, double rings or multiple rings can be fused rings, spiro rings, bridged rings or these in a combined form, double rings or multiple rings can be comprised of one or more aromatic rings, but the overall ring system is not aromatic, and the binding sites can be on aromatic rings or non-aromatic rings. Customarily, cycloalkyls contain 3 to 20 carbon atoms, and may contain 3-8 carbon atoms, and furthermore even contain 3-6 carbon atoms; where single monocyclic cycloalkyls are concerned, these contain 3-15 carbon atoms, or 3-10 carbon atoms, or 3-8 carbon atoms, or 3-6 carbon atoms; where double ring or multiple ring cycloalkyls are concerned, these contain 5-12 carbon atoms, or contain 5-11 carbon atoms, or contain 6-10 carbon atoms.

“Carbon ring” or “carbocyclic” refers to a substituted or un-substituted carbon ring group, and includes single carbon rings or double ring bridged rings, double fused rings, double spiro rings and multi-membered rings with three or more rings, customarily having 3 to 14 carbon atoms, preferably having 3-12 carbon atoms, and more preferably having 6-8 carbon atoms or having 3-6 carbon atoms. In the non-restrictive embodiments, single ring carbon rings include cyclopropyl, cyclobutyl, cyclopentyl, cyclohexyl, cycloheptyl or phenyl etc., double ring bridged rings, double ring fused rings, double ring spiro rings rings etc.

“Heterocycle” or “heterocyclyl” refers to a substituted or un-substituted, saturated or unsaturated, aromatic ring or non-aromatic ring, and unless specifically stated, includes 1 to 5 heteroatoms selected from N, O or S, preferably containing 1 to 4 heteroatoms, more preferably containing 1-3 heteroatoms, including monocyclic heterocycles, double ring bridged heterocycles, double ring fused heterocycles and double ring spiro heterocycles, as well as heterocycles with three or more rings etc. Preferably these are 3 to 15 membered heterocycles, more preferably these are 4-14 membered heterocycles, and more preferably these are 4-10 membered heterocycles or 5-12 membered heterocycles, and even more preferably 5-8 membered heterocycles or 5-6 membered heterocycles. Heterocycles are preferably saturated heterocycles, such as 5-12 membered saturated heterocycles, and more preferably are 5-8 membered saturated heterocycles, 7 membered saturated heterocycles or 5-6 membered saturated heterocycles. The N or S ring atom of the heterocyclyl can be oxidised to various oxidised states. The heterocyclyl can bind to a heteroatom or carbon atom, non-restrictive embodiments include glycidyl, azacyclic propyl, oxyheterocyclic butyl, azacyclic butyl, 1,3-dioxypentyl, 1,4-dioxypentyl, 1,3-dioxane, piperazinyl, azacyclic heptyl, pyridyl, furyl, thienyl, pyranyl, N-alkylpyrrolyl, pyrimidyl, pyrazinyl, pyrazolyl, pyridazinyl, imidazolyl, piperidyl, piperidinyl, morpholinyl, thio-morpholinyl, 1,3-dithianyl, dihydrofuryl, dihydropyranyl, dithiopentyl, tetrahydrofuryl, tetrahydropyrrolyl, tetrahydroimidazolyl, oxazolyl, dihydrooxazolyl, tetrahydrooxazolyl, tetrahydrothiazolyl, tetrahydropyranyl, benzoimidazolyl, benzopyridyl, pyrrolopyridyl, benzodihydrofuryl, azabicyclo[3.2.1]octyl, azabicyclo[5.2.0]nonyl, oxatricyclo[5.3.1.1] dodecyl, azaadamantane and oxaspiro[3.3]heptane,

“Ortho-heterocyclyl” refers to a substituted or un-substituted, saturated or unsaturated, aromatic or non-aromatic divalent heterocyclylic group.

“Aryl” refers to an aromatic group, including 5- and 6-membered monocyclic aromatic groups containing 0 to 4 N, S or O atoms, and a multiple ring system possessing at least one aromatic ring. In concept, this includes aromatic carbon rings and heteroaromatic rings, such as phenyl, pyrrole, furan, thiophene, thiazole, isothiazole, imidazole, triazole, tetrazole, pyrazol, oxazole, isooxazole, pyridine, pyrazine, pyridazine and pyridine etc. multiple ring aromatic groups (tricyclo or bicyclo) such as naphthalene, benzoxazole, benzodioxazole, benzothiazole, benzoimidazole, benzothiophene, methylenedioxybenzene, quinoline, isoquinoline, naphthyridine, indole, benzofuran, purine, benzofuran, deazapurine or indolizine. The aryls in a ring structure that possess heteroatoms are also known as “aromatic heterocycles”, “heteroaryls” or “heteroaromatic rings”.

“Spiro ring” refers to multiple ring groups where there is a shared carbon atom (referred to as the spiro atom) between the rings, these can include 0 to 1 or more double-bonds or triple-bonds, they can contain 0 to 5 heteroatoms selected from N, O, S, P or Si. Customarily, spiro rings are 6 to 14 membered rings, or 6 to 12 membered rings, or 6 to 10 membered rings. Customarily, spiro rings are spiro[3.3] (representing 3 membered ring spiro 3 membered ring), spiro[3.4], spiro[3.5], spiro[3.6], spiro[4.4], spiro[4.5], spiro[4.6], spiro[5.5], or spiro[5.6]

“Fused ring” refers to a multiple ring group where two adjacent atoms and one chemical bond are shared by rings, and these may contain one or more double-bonds or triple-bonds, fused rings can contain 0 to 5 heteroatoms of N, S, O, P or Si and their oxidised states. Customarily fused rings are 5 to 30 membered rings, or 5 to 14 membered rings, or 5 to 12 membered rings, or 5 to 10 membered rings. Customarily, fused rings are 3,4 cyclo (indicating a three membered ring and a four membered ring forming a fused ring, which according to the IUPC naming rules may consist of a fused ring with a three membered ring as the base ring or a four membered ring as the base ring, this also applies to the following) 3,5 cyclo, 3,6 cyclo, 4,4 cyclo, 4,5 cyclo, 4,6 cyclo, 5,5 cyclo, 5,6 cyclo and 6,6 cyclo. Non-restrictive examples of fused rings include purine, quinoline, isoquinoline, benzopyran, benzofuran, benzothiophene, said fused rings optionally substituted by any substitution group.

“Bridge ring” refers to two rings sharing two non-adjacent ring atoms, these can contain 1 or more double-bonds or triple-bonds. Bridge rings can contain 0 to 5 heteroatoms selected from N, S, O, P or Si and their oxidised states. Customarily bridged rings have 5 to 20 ring atoms, or 5 to 14, or 5 to 12 or 5 to 10.

“Substitution” or “substitution group” unless specifically stated otherwise, refers to arbitrary substitution occurring at any chemically feasible position, the number of substitution groups satisfying chemical bonding rules. Examples of substitution groups include but are not limited to: C₁₋₆alkyl, C₂₋₆alkenyl, C₂₋₆alkynyl, C₃₋₈heteroalkyl, C₅₋₁₂aryl, 5-12 membered heteroaryl, hydroxyl, C₁₋₆alkoxy, C₅₋₁₂aryloxy, thiol, C₁₋₆alkylthio, cyano, halogen, C₁₋₆alkylthio carbonyl, C₁₋₆alkanamine formyl, N-carbazochrome, nitryl, methylsilyl, ortho-sulfonyl, sulfonyl, sulfoxide, halogenated C₁₋₆alkyl, halogenated C₁₋₆alkoxy, azyl, phosphonic acid, —CO₂ (C₁₋₆alkyl), —OC(═O) (C₁₋₆alkyl), —OCO₂ (C₁₋₆ alkyl), —C(═O) NH₂, —C(═O) N(C₁₋₆alkyl) 2, —OC(═O) NH(C₁₋₆alkyl), —NHC(═O) (C₁₋₆ alkyl), —N(C₁₋₆alkyl) C(═O) (C₁₋₆alkyl), —NHCO₂ (C₁₋₆alkyl), —NHC(═O) N(C₁₋₆ alkyl) 2, —HC(═O) NH(C₁₋₆alkyl), —NHC(═O) NH₂, —NHSO₂ (C₁₋₆alkyl), —SO₂N(C₁₋₆alkyl) 2, —SO₂NH(C₁₋₆alkyl), —SO₂NH₂ and —SO₂C₁₋₆alkyl etc.

“Pharmaceutically acceptable salt” refers to compounds of the present invention maintaining free acid or free alkali bioavailability and characteristics, and said free acid is obtained by reaction of a non-toxic inorganic alkali or organic alkali, or said free alkali is obtained by reaction of a non-toxic inorganic acid or organic acid.

“Stereoisomer” refers to an isomer occurring in molecule atoms due to different spatial orientation modes, including cis-trans isomers, enantiomers and conformational isomers.

“Solvate” refers to stoichiometric or non-stoichiometric substances resulting from intermolecular combining of compounds of the present invention or their salts due to noncovalent forces. If the solvent is water, these are hydrates.

“Co-crystal” refers to crystals formed due to the effects of hydrogen bonds or noncovalent bonds forming due to binding between active pharmaceutical ingredients (API) and a co-crystal former (CCF), whereby the pure state of the API and CCF is a solid at room temperature, and there is a fixed stoichiometric ratio between each ingredient. Co-crystals are a type of multi-component crystal, and as such they include a binary co-crystal formed between the neutral solid and the salt or solvate.

As used herein, the symbol

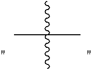

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

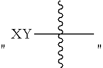

indicates that the chemical entity “XY” is bonded to another chemical entity via the point of attachment bond. Furthermore, the specific point of attachment to the non-depicted chemical entity can be specified by inference. For example, the compound CH₃—R^{L}, wherein R_{L} is H or

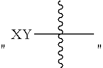

infers that when R^{L} is “XY”, the point of attachment bond is the same bond as the bond by which R^{L} is depicted as being bonded to CH₃.

The compounds of Formula (I), (Ia) and (Ib), and their pharmaceutically acceptable salts are dipeptidyl peptidase 1 (DPP1 or cathepsin C) inhibitors, and thus may be used in any disease area where DPP1 plays a role. As such, in one aspect of the invention, a method of treatment is provided. The method of treatment, in one embodiment, comprises, administering to a subject in need of, for an administration period, a composition comprising an effective amount of a compound of Formula (I), (Ia) or (Ib), or a pharmaceutically acceptable salt thereof. In one embodiment, a subject's symptom(s) or clinical outcome are improved during the administration period or subsequent to the administration period, as compared to the respective symptom(s) or clinical outcome measured prior to the administration period.

“Prior to the administration period”, as used herein, refers to a time period of from about 28 days prior to the initial administration of the pharmaceutical composition or compound of Formula (I) provided herein, to immediately prior to the initial administration of the pharmaceutical composition or compound of Formula (I). “Immediately prior to the administration period” in one embodiment, is from about 24 hours prior to about 1 minute prior to the initial administration of the pharmaceutical composition or compound of Formula (I) provided herein.

In one embodiment, prior to the administration period is from about 28 days prior to immediately prior to the administration period. In another embodiment, prior to the administration period is from about 21 days prior to immediately prior to the administration period. In another embodiment, prior to the administration period is from about 14 days prior to immediately prior to the administration period. In even another embodiment, prior to the administration period is from about 10 days prior to immediately prior to the administration period. In yet even another embodiment, prior to the administration period is from about 7 days prior to immediately prior to the administration period. In even yet another embodiment, prior to the administration period is from about 4 days prior to immediately prior to the administration period.

In one embodiment, “prior to the administration period” is from about 28 days prior to about 1 day prior to the administration period. In another embodiment, prior to the administration period is from about 21 days prior to about 1 day prior to the administration period. In another embodiment, prior to the administration period is from about 14 days prior to about 1 day prior to the administration period. In even another embodiment, prior to the administration period is from about 10 days prior to about 1 day prior to the administration period. In yet even another embodiment, prior to the administration period is from about 7 days prior to about 1 day prior to the administration period. In even yet another embodiment, prior to the administration period is from about 4 days prior to about 1 day prior to the administration period.

As used herein, the terms “treatment”, “treating,” “ameliorating” and variations thereof, are used interchangeably. These terms refer to an approach for obtaining beneficial or desired results including but not limited to a therapeutic benefit and/or a prophylactic benefit. Therapeutic benefit refers to any therapeutically relevant improvement in or effect on one or more diseases, conditions, or symptoms under treatment. The term “treating” in one embodiment, includes: (1) preventing or delaying the appearance of clinical symptoms of the state, disorder or condition developing in the patient that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); (3) relieving the condition (for example, by causing regression, or reducing the severity of the state, disorder or condition or at least one of its clinical or subclinical symptoms).

The term “effective amount” or “therapeutically effective amount” refers to the amount of an agent that is sufficient to achieve an outcome, for example, to effect beneficial or desired results. The therapeutically effective amount may vary depending upon one or more of: the subject and disease condition being treated, the weight and age of the subject, the severity of the disease condition, the manner of administration and the like.

The terms “subject,” “individual,” and “patient” are used interchangeably herein to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human. A subject's tissues, cells, or derivatives thereof, obtained in vivo or cultured in vitro are also encompassed. A human subject may be an adult, a teenager, a child (2 years to 14 years of age), an infant (1 month to 24 months), or a neonate (up to 1 month). In some embodiments, the adults are seniors about 65 years or older, or about 60 years or older.

In one aspect, the present invention provides a method of treating a disorder associated with altered DPP1 activity in a subject in need of treatment, comprising administering to the subject for an administration period, a composition comprising an effective amount of a compound of Formula (I), or a pharmaceutically acceptable salt thereof:

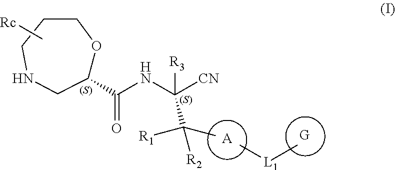

wherein,

G is a 5-12 membered carbon ring, a 5-12 membered monocyclic heterocycle containing 1-3 heteroatoms selected from N, S or O or a fused ring of formula

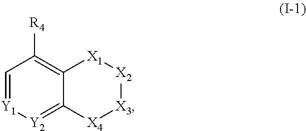

L₁ bonds G by replacing any hydrogen atom on G, and is a bond, C₁₋₃ alkylene, —NH—, —N(C₁₋₄alkyl)-, —O—, —S—, C₂₋₆ortho-alkenyl, C₂₋₆ortho-alkynyl, —CO— or —CONH—, wherein the alkylene, ortho-alkenyl or ortho-alkynyl is optionally substituted with 1-3 halogen, C₁₋₄ alkyl, cyano, hydroxyl, NH₂ and —COOH groups;
R₁, R₂ and R₃ are independently selected from H, deuterium, halogen, C₁₋₄alkyl, C₁₋₄alkoxy, C₂₋₆alkenyl, C₂₋₆alkenyl, C₂₋₆alkynyl, C₃₋₆cycloalkyl and a 4-7 membered heterocycle containing 1-3 heteroatoms selected from N, S or O, the alkyl, alkoxy, alkenyl, alkynyl, cycloalkyl and heterocycle having arbitrarily undergone substitution with 1-3 groups selected from halogen, C₁₋₄alkyl, cyano, hydroxyl, NH₂ and COOH; alternatively, R₁ and R₂ form a C₃₋₆cycloalkyl or a 4-7 membered heterocycle containing 1-3 heteroatoms selected from N, S or O, wherein the cycloalkyl or heterocycle is optionally substituted with 1-3 groups selected from ═O, halogen, cyano, hydroxyl, NH₂, COOH, C₁₋₄ alkyl, C₂₋₆ alkenyl, C₂₋₆alkynyl and C₃₋₆cycloalkyl;
Y₁ and Y₂ are each independently selected from CR₄ or N;
each R₄ is independently selected from H, deuterium, halogen, C₁₋₄ alkyl, C₁₋₄ alkoxy, C₂₋₆ alkenyl, C₂₋₆ alkynyl, C₃₋₆cycloalkyl, cyano, hydroxyl, NH₂, NHC₁₋₄ alkyl, N(C₁₋₄ alkyl) 2, COOH, COC₁₋₄ alkyl, COOC₁₋₄ alkyl, CONHC₁₋₄ alkyl, CON(C₁₋₄ alkyl) 2, NHCOC₁₋₄ alkyl, and a 4-7 membered heterocycle containing 1-3 heteroatoms selected from N, S or O; wherein the alkyl, alkenyl, alkynyl, cycloalkyl and heterocycle groups within R₄ are optionally substituted with 1-3 groups selected from deuterium, halogen, cyano, hydroxyl, NH₂ and COOH;
X₁, X₂, X₃ and X₄ are each independently selected from a bond, NR₅, O, CR₆R₇, S, S(O) and S(O) 2, and wherein at most one of X₁, X₂, X₃ and X₄ is a bond;
Rc is ═O, halogen, cyano, hydroxyl, NH₂, —COOH, C₁₋₄ alkyl, halogenated C₁₋₄ alkyl, hydroxyl C₁₋₄ alkyl, C₁₋₄ alkoxy, C₁₋₆ alkoxyalkyl or halogenated C₁₋₄ alkoxy;
each R₅ is independently selected from H, C₁₋₄ alkyl, —COC₁₋₄ alkyl, C₂₋₆ alkenyl, C₂₋₆ alkynyl and C₃₋₆cycloalkyl; wherein the alkyl, alkenyl, alkynyl and cycloalkyl is optionally substituted with 1-3 groups selected from deuterium, halogen, cyano, hydroxyl, NH₂ and COOH;
R₆ and R₇ are each independently selected from H, deuterium, halogen, C₁₋₄ alkyl, C₁-₄alkoxy, C₂₋₆ alkenyl, C₂₋₆ alkynyl, C₃₋₆cycloalkyl, cyano, hydroxyl, NH₂, COOH, and a 4-7 membered heterocycle containing 1-3 heteroatoms selected from N, S or O, wherein the alkyl, alkoxy, alkenyl, alkynyl, cycloalkyl and heterocycle is optionally substituted with 1-3 groups arbitrarily selected from deuterium, halogen, cyano, hydroxyl, NH₂ and —COOH;
alternatively, R₆ and R₇ form ═O;
alternatively, two R₅ on the atoms in adjacent rings or two R₆ on the atoms in adjacent rings within X₁, X₂, X₃ and X₄, or the R₅ and R₆ of atoms on adjacent rings and the atoms binding to them form double-bonds;
alternatively, the R₆ and R₇ on the same carbon atom form a C₃₋₁₂ carbon ring with the carbon atoms linked to them or a 4-7 membered heterocycle containing 1-3 heteroatoms selected from N, S or O, said carbon ring or heterocycle having arbitrarily undergone substitution with 1-3 groups selected from ═O, halogen, cyano, hydroxyl, NH₂, —COOH, C₁₋₄ alkyl, C₂₋₆ alkenyl, C₂₋₆ alkynyl and C₃₋₆ cycloalkyl;
Z is CH or N;

A is

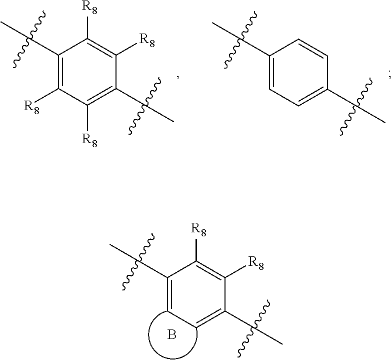

a hexatomic heteroaryl or 5-7 membered non-aromatic monocyclic heterocycle, wherein the heteroaryl and non-aromatic monocyclic heterocycle contains 1-3 heteroatoms selected from N, S or O;
B is a C₄₋₆ carbon ring or a 5-6 membered heterocycle containing 1-3 heteroatoms selected from N, S or O; and
each R₈ is independently selected from H, ═O, deuterium, halogen, C₁₋₄ alkyl, C₁₋₄ alkoxy, SC₁₋₄ alkyl, C₂₋₆ alkenyl, C₂₋₆ alkynyl, cyano, hydroxyl, —COOH, NH₂ and C₃₋₆ cycloalkyl; said alkyl, alkoxy, alkenyl, alkynyl or cycloalkyl having arbitrarily undergone substitution with 1-₃ groups selected from deuterium, halogen, cyano, hydroxyl, NH₂ and —COOH.

In one embodiment of a compound of Formula (I), or a pharmaceutically acceptable salt thereof, the compound is a compound of Formula (Ia), or a pharmaceutically acceptable salt thereof:

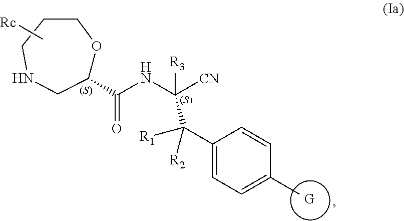

wherein,
R₁, R₂, R₃ and G are defined above for Formula (I); and
Rc is ═O, halogen, cyano, hydroxyl, NH₂, —COOH, C₁₋₄ alkyl, halogenated C₁₋₄ alkyl, hydroxyl C₁₋₄ alkyl, C₁₋₄ alkoxy, C₁₋₆ alkoxyalkyl or halogenated C₁₋₄ alkoxy.

In one embodiment of a compound of Formula (I), or a pharmaceutically acceptable salt thereof, the compound is a compound of Formula (Ib), or a pharmaceutically acceptable salt thereof:

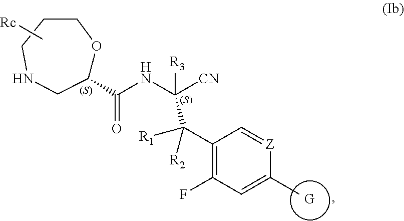

wherein,
R₁, R₂, R₃, Z and G are defined above for Formula (I); and
Rc is ═O, halogen, cyano, hydroxyl, NH₂, —COOH, C₁₋₄ alkyl, halogenated C₁₋₄ alkyl, hydroxyl C₁₋₄ alkyl, C₁₋₄ alkoxy, C₁₋₆ alkoxyalkyl or halogenated C₁₋₄ alkoxy.

A person skilled in the art would be capable of preparing the compounds of Formula (I) based on known organic synthesis technology, and the starting materials are commercially available chemicals and (or) compounds mentioned in chemical documents. “Commercially available chemicals” are those that can be obtained from actual commercial sources, such suppliers including: Titan, Energy Chemical, Shanghai Demo, Chengdu Chron Chemicals, Accela ChemBio Co., Ltd., Nanjing PharmaBlock, WuXi AppTec and J&K Scientific etc.

Reference publications and monographs in this field have described in detail the synthesis of the reagents used in preparation of the compounds described in this text, or provide articles that describe such preparation methods for reference. These reference books and monographs include: “Synthetic Organic Chemistry”, John Wiley & Sons, Inc., New York; S. R. Sandler et al., “Organic Functional Group Preparations,” 2^{nd} Ed., Academic Press, New York, 1983; H. O. House, “Modern Synthetic Reactions”, 2^{nd} Ed., W. A. Benjamin, Inc. Menlo Park, Calif. 1972; T. L. Gilchrist, “Heterocyclic Chemistry”, 2^{nd} Ed., John Wiley & Sons, New York, 1992; J. March, “Advanced Organic Chemistry: Reactions, Mechanisms and Structure”, 4th Ed., Wiley-Interscience, New York, 1992; Fuhrhop and Penzlin. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2^{nd} Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2^{nd} Edition (1993) Wiley-Interscience, ISBN: 0-471-57456-2; “Industrial Organic Chemicals: Starting Materials and Intermediates: An Ullmann's Encyclopaedia” (1999) John Wiley & Sons, ISBN: 3-527-₂₉₆₄₅-X, in 8 volumes; “Organic Reactions” (1942-2000) John Wiley & Sons, in over 55 volumes; and “Chemistry of Functional Groups” John Wiley & Sons, in 73 volumes.

By accessing the CAS index of known chemical substances prepared by the American Chemical Society, it is possible to selectively identify specific and similar reagents, these indexes are accessible in the majority of public libraries, university libraries and online. Known chemicals in the list that cannot be purchased commercially can alternatively be tailor-made by chemical synthesis laboratories, and many of the standard chemical suppliers (for instance, those listed above) provide a tailor-made synthesis service. The reference publication for preparation and selection of the pharmaceutical salts of the compounds described in this text is P. H. Stahl & C. G. Wermuth “Handbook of Pharmaceutical Salts”, Verlag Helvetica Chimica Acta, Zurich, 2002.

In one embodiment of a compound of Formula (I), (Ia) or (Ib), for use in a method provided herein, G is

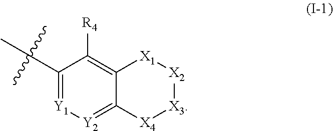

In one embodiment of a compound of Formula (I), (Ia) or (Ib), G is

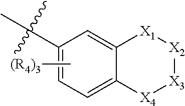

In one embodiment of a compound of Formula (I), (Ia) or (Ib), G is

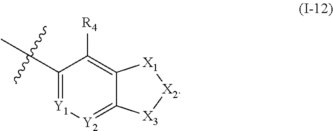

In a preferred embodiment, G is

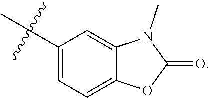 [SMILES: *c1ccc2oc(=O)n(C)c2c1]

In a further embodiment, Rc is halogen, C₁₋₂alkyl or C₁₋₂ alkoxy. In a further embodiment, Rc is methoxy or ethoxy. In yet another embodiment, Rc is hydroxyl.

In one embodiment, G is a substituted cyclopentane, cyclohexane, cycloheptane or benzene,

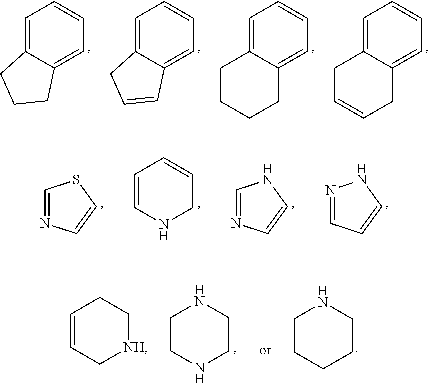

In one embodiment, G is

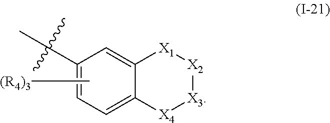

In one embodiment of a method of treatment provided herein, in the compound of Formula (I), (Ia) or (Ib), or a pharmaceutically acceptable salt thereof, G is

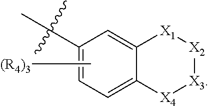

In a further embodiment, G is one of the following:

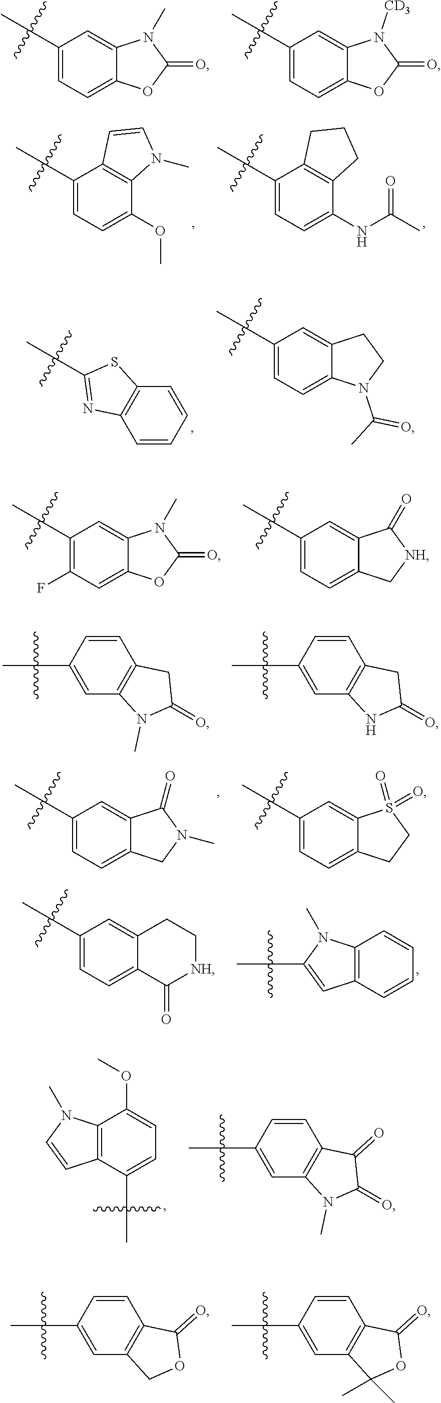

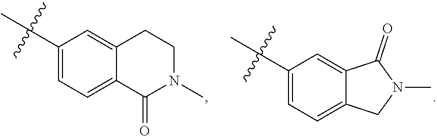 [SMILES: *c1ccc2c(c1)CCN(C)C2=O ; *c1ccc2c(c1)C(=O)N(C)C2]

In one embodiment, G is

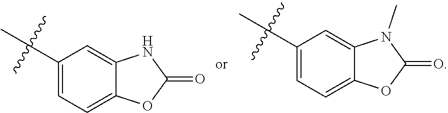 [SMILES: *c1ccc2oc(=O)[nH]c2c1 ; *c1ccc2oc(=O)n(C)c2c1]

In a further embodiment, Rc is halogen, C₁₋₂alkyl or C₁₋₂ alkoxy. In a further embodiment, Rc is methoxy or ethoxy. In yet another embodiment, Rc is hydroxyl.

In another embodiment, G is

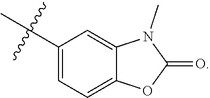 [SMILES: *c1ccc2oc(=O)n(C)c2c1]

In a further embodiment, R₁, R₂ and R₃ are each H. In even a further embodiment, Rc is hydroxyl, C₁₋₄ alkyl, or C₁₋₄ alkoxy.

In some embodiments of the method of treatment provided herein, the G moiety of Formula (I), (Ia) or (Ib) is substituted by one or more R_{G} groups. In a further embodiment, each R_{G} is independently selected from deuterium, SF₅, ═O, halogen, cyano, hydroxyl, NH₂, —COOH, C₁₋₄ alkyl, C₁₋₄alkoxy, C₃₋₆cycloalkyloxy, C₃₋₆cycloalkyl, C₂₋₆alkenyl, C₂₋₆alkynyl, —NHC₁₋₄alkyl, —N(C₁₋₄alkyl) 2, —COC₁₋₄ alkyl, —COOC₁₋₄ alkyl, —CONH₂, —CONHC₁₋₄alkyl, —CONHC₃₋₆cycloalkyl, —CON(C₁₋₄alkyl) 2, —NHCOC₁₋₄alkyl, —NHCOC₃₋₆cycloalkyl, —P(O) (C₁₋₄ alkyl) 2, —S(O) C₁₋₄ alkyl, —S(O) 2 C₁₋₄alkyl, —S(O) 2 C₃₋₆cycloalkyl, —S(O) NH₂, —S(O) NHC₁₋₄ alkyl, —S(O) N(C₁₋₄ alkyl) 2, —S(O)₂NH₂ and a 4-7 membered heterocycle containing 1-3 heteroatoms selected from N, S or O, said R_{G} preferably having arbitrarily undergone substitution with 1-3 groups selected from deuterium, C₁₋₄alkyl, halogenated C₁₋₄alkyl, hydroxyl C₁₋₄alkyl, —CONH₂, NH₂, C₁₋₆ alkoxy, hydroxyl, —COOH, halogen and a 5-7 membered ring containing 1-3 heteroatoms selected from N, S or O, and preferably, the 5-7 membered heterocycle has arbitrarily undergone substitution by 1-2-O, halogen, cyano, C₁₋₄ alkyl or halogenated C₁₋₄ alkyl.

In one embodiment, G is substituted by one or more R_{G} groups. In a further embodiment, each R_{G} is independently selected from F, Cl, Br, I, methyl, ethyl, propyl, SF₅ and CN; said methyl, ethyl and propyl optionally being substituted with 1-3 groups selected from F, Cl, Br and I.

In a further embodiment, Rc is halogen, C₁₋₂alkyl or C₁₋₂ alkoxy. In a further embodiment, Rc is methoxy or ethoxy. In yet another embodiment, Rc is hydroxyl.

In one embodiment, R₁, R₂ and R₃ are each independently selected from H, deuterium, halogen, C₁₋₄ alkyl, C₁₋₄ alkoxy, C₂₋₆ alkenyl and C₂₋₆ alkynyl, said alkyl, alkoxy, alkenyl and alkynyl having arbitrarily undergone substitution with 1-3 groups selected from halogen, C₁₋₄alkyl, cyano, hydroxyl, NH₂ and COOH.

In a preferred embodiment, each of R₁, R₂ and R₃ are H. In a further embodiment, where a compound of Formula (Ib) is employed, Z is CH. In even a further embodiment, Rc is halogen, C₁₋₂alkyl or C₁₋₂ alkoxy. In yet even a further embodiment, G is

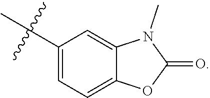 [SMILES: *c1ccc2oc(=O)n(C)c2c1]

In another embodiment, R₁ and R₂ form a C₃₋₆ cycloalkyl or a 4-7 membered heterocycle containing 1-3 heteroatoms selected from N, S or O with the carbon atoms they are linked with, said cycloalkyl or heterocycle having arbitrarily undergone substitution with 1-3 groups selected from halogen, cyano, hydroxyl, NH₂, COOH and C₁₋₄ alkyl.

In yet another embodiment, R₁, R₂ and R₃ are each independently selected from H, deuterium, halogen, C₁₋₄alkyl or C₁₋₄alkoxy, said alkyl and alkoxy having arbitrarily undergone substitution with 1-3 groups selected from halogen, C₁₋₄alkyl, cyano, hydroxyl, NH₂ and COOH.

In even another embodiment, R₁ and R₂ form a C₃₋₆cycloalkyl with the carbon atoms they are linked with, said cycloalkyl having arbitrarily undergone substitution with 1-3 groups selected from halogen, cyano, hydroxyl, NH₂, COOH and C₁₋₄ alkyl;
in certain specific embodiments R₁, R₂ and R₃ are each independently selected from H, deuterium, F, Cl, Br, methyl, ethyl, methoxy or ethoxy, said methyl, ethyl, methoxy or ethoxy having arbitrarily undergone substitution with 1-3 groups selected from F, Cl, Br, cyano, hydroxyl and NH₂;
In one embodiment of a compound of Formula (I), or a pharmaceutically acceptable salt thereof, for use in one of the methods provided herein, L₁ is a bond. In a further embodiment, Rc is hydroxyl, C₁₋₄ alkyl, or C₁₋₄ alkoxy. In even a further embodiment, Rc is hydroxyl, methoxy, ethoxy, methyl, ethyl or propyl.

In one embodiment of a compound of Formula (I), or a pharmaceutically acceptable salt thereof, for use in one of the methods provided herein, L₁ is a bond; G is

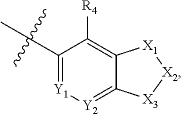

Y₁ and Y₂ are both independently selected from CR₄ or N; X₁ is N, X₂ is C(O) and X₃ is O.

In one embodiment of a compound of Formula (I), or a pharmaceutically acceptable salt thereof, for use in one of the methods provided herein, A is

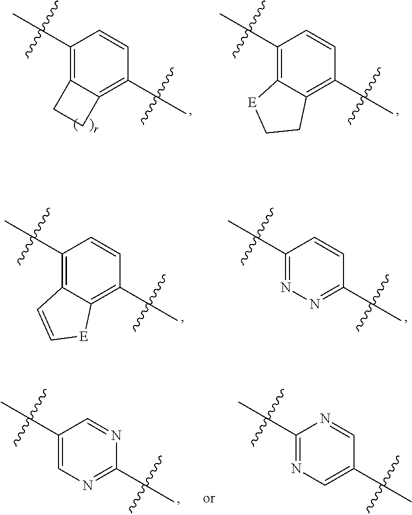

wherein, r is an integer from 1-3 and E is NH, S or O. In a further embodiment, A is

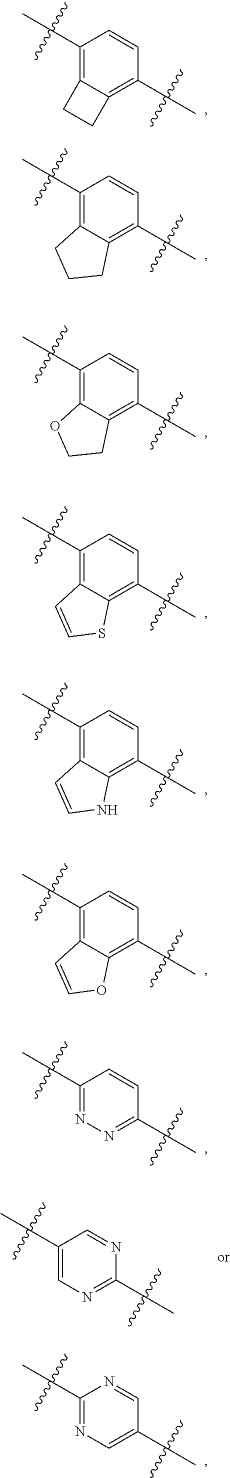

In one preferred embodiment, A is

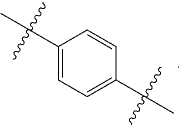 [SMILES: *c1ccc(*)cc1]

In one embodiment of a compound of Formula (I), or a pharmaceutically acceptable salt thereof, for use in one of the methods provided herein, A is

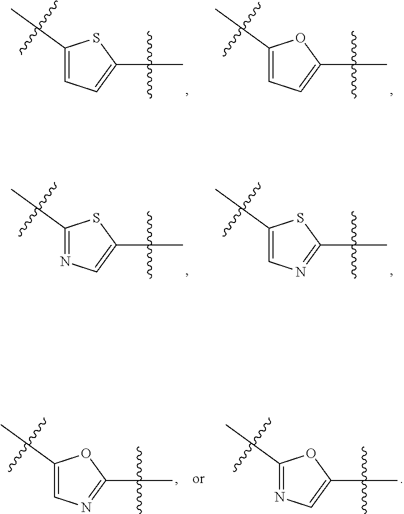 [SMILES: *c1ccc(*)s1 ; *c1ccc(*)o1 ; *c1cnc(*)s1 ; *c1cnc(*)s1 ; *c1cnc(*)o1 ; *c1cnc(*)o1]

In one embodiment, the compound for use in one of the methods provided herein is selected from one of the following structures:

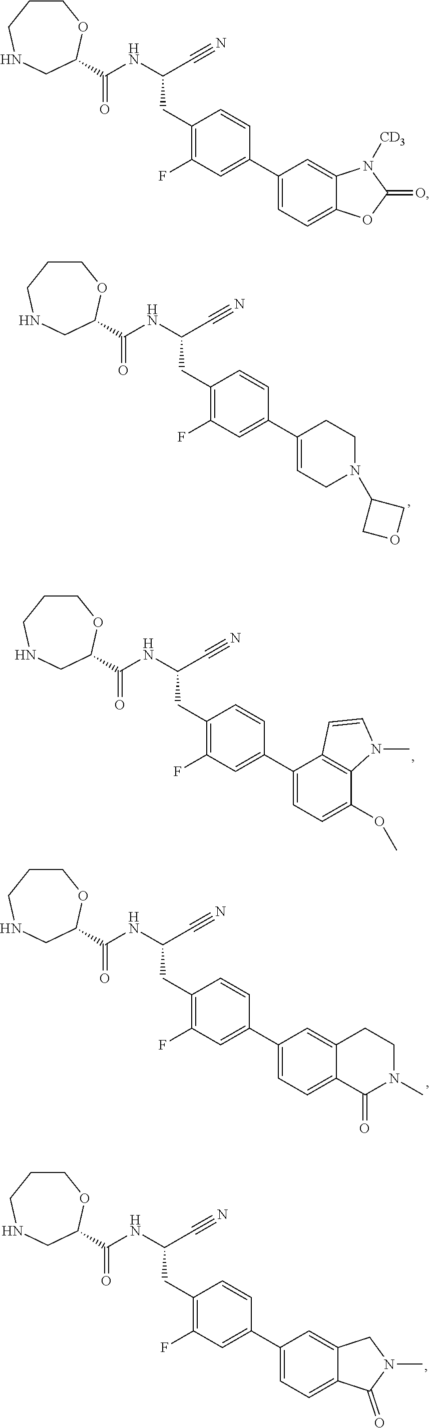 [SMILES: [2H]C([2H])([2H])n1c(=O)oc2ccc(-c3ccc(C[C@@H](C#N)NC(=O)[C@@H]4CNCCCO4)c(F)c3)cc21 ; N#C[C@H](Cc1ccc(C2=CCN(C3COC3)CC2)cc1F)NC(=O)[C@@H]1CNCCCO1 ; COc1ccc(-c2ccc(C[C@@H](C#N)NC(=O)[C@@H]3CNCCCO3)c(F)c2)c2ccn(C)c12 ; CN1CCc2cc(-c3ccc(C[C@@H](C#N)NC(=O)[C@@H]4CNCCCO4)c(F)c3)ccc2C1=O ; CN1Cc2cc(-c3ccc(C[C@@H](C#N)NC(=O)[C@@H]4CNCCCO4)c(F)c3)ccc2C1=O]

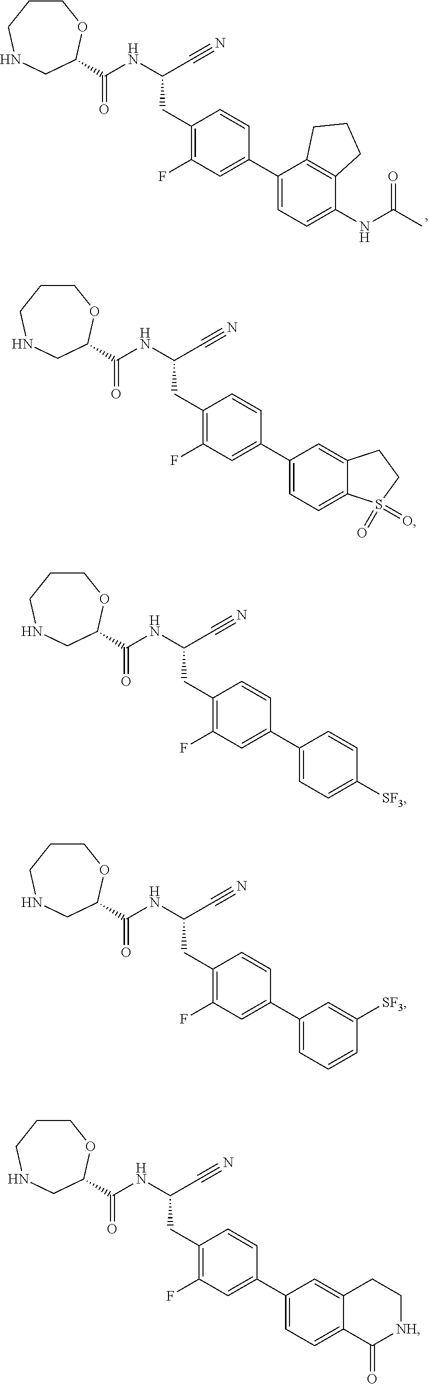

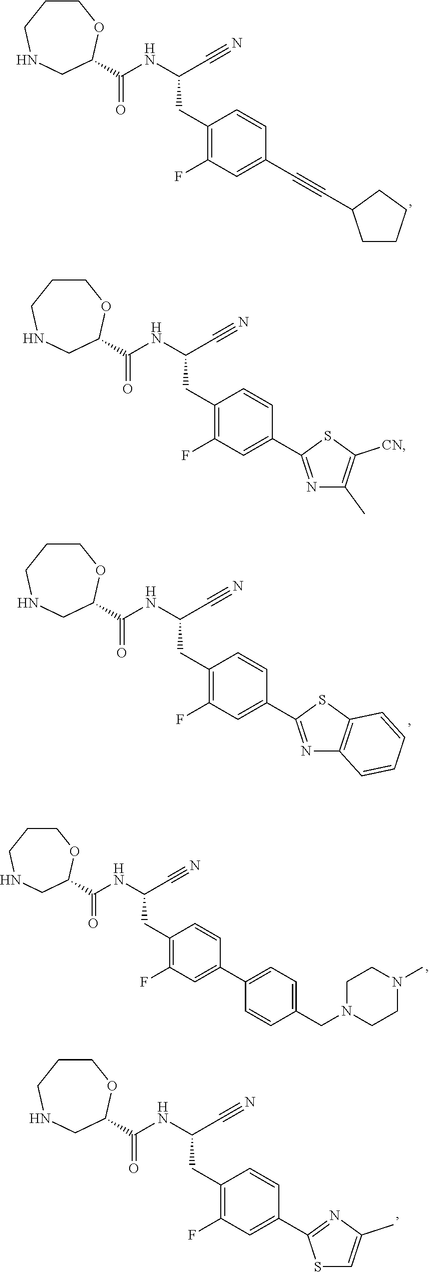 [SMILES: N#C[C@H](Cc1ccc(C#CC2CCCC2)cc1F)NC(=O)[C@@H]1CNCCCO1 ; Cc1nc(-c2ccc(C[C@@H](C#N)NC(=O)[C@@H]3CNCCCO3)c(F)c2)sc1C#N ; N#C[C@H](Cc1ccc(-c2nc3ccccc3s2)cc1F)NC(=O)[C@@H]1CNCCCO1 ; CN1CCN(Cc2ccc(-c3ccc(C[C@@H](C#N)NC(=O)[C@@H]4CNCCCO4)c(F)c3)cc2)CC1 ; Cc1csc(-c2ccc(C[C@@H](C#N)NC(=O)[C@@H]3CNCCCO3)c(F)c2)n1]

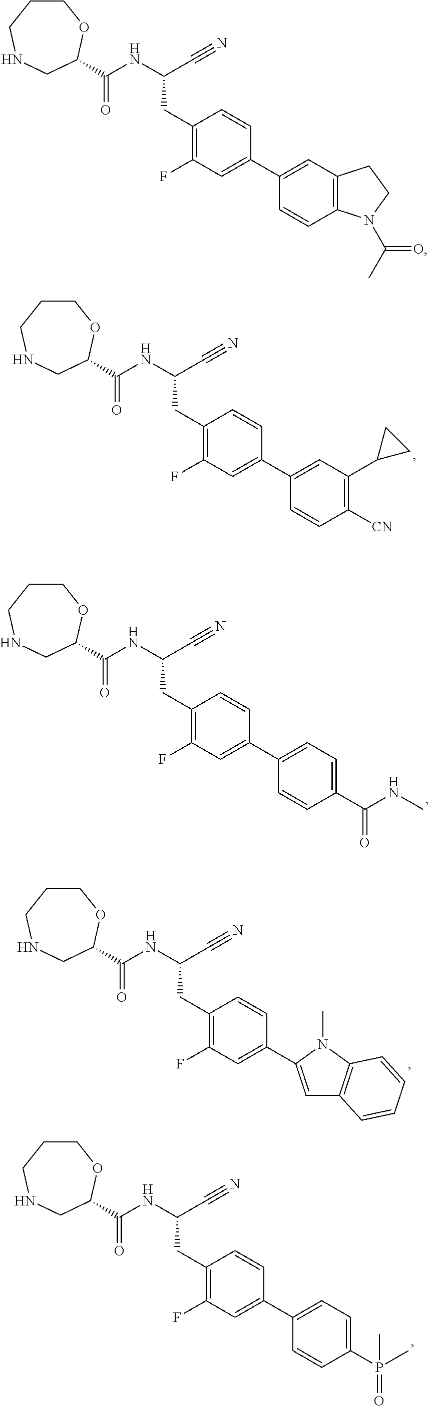 [SMILES: CC(=O)N1CCc2cc(-c3ccc(C[C@@H](C#N)NC(=O)[C@@H]4CNCCCO4)c(F)c3)ccc21 ; N#Cc1ccc(-c2ccc(C[C@@H](C#N)NC(=O)[C@@H]3CNCCCO3)c(F)c2)cc1C1CC1 ; CNC(=O)c1ccc(-c2ccc(C[C@@H](C#N)NC(=O)[C@@H]3CNCCCO3)c(F)c2)cc1 ; Cn1c(-c2ccc(C[C@@H](C#N)NC(=O)[C@@H]3CNCCCO3)c(F)c2)cc2ccccc21 ; CP(C)(=O)c1ccc(-c2ccc(C[C@@H](C#N)NC(=O)[C@@H]3CNCCCO3)c(F)c2)cc1]

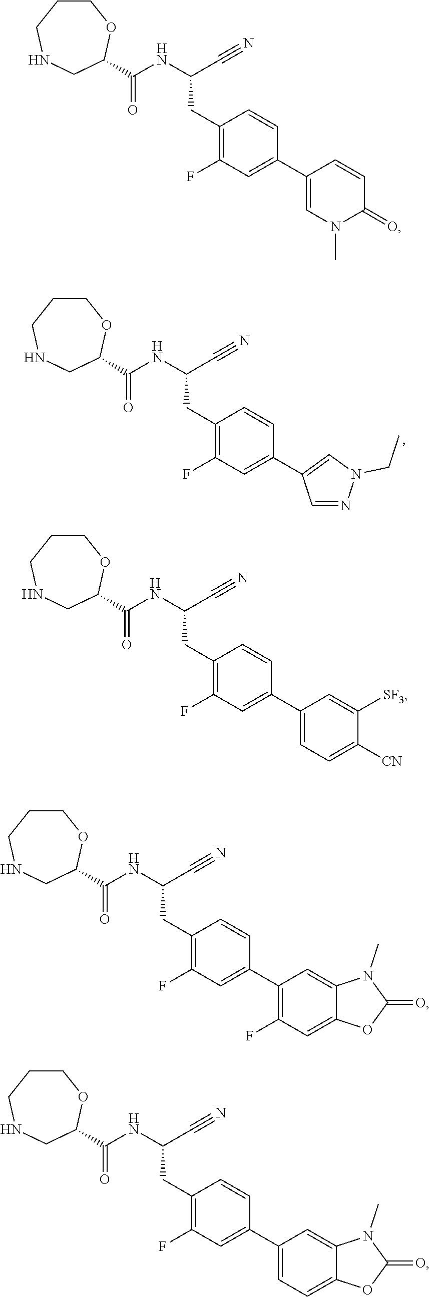

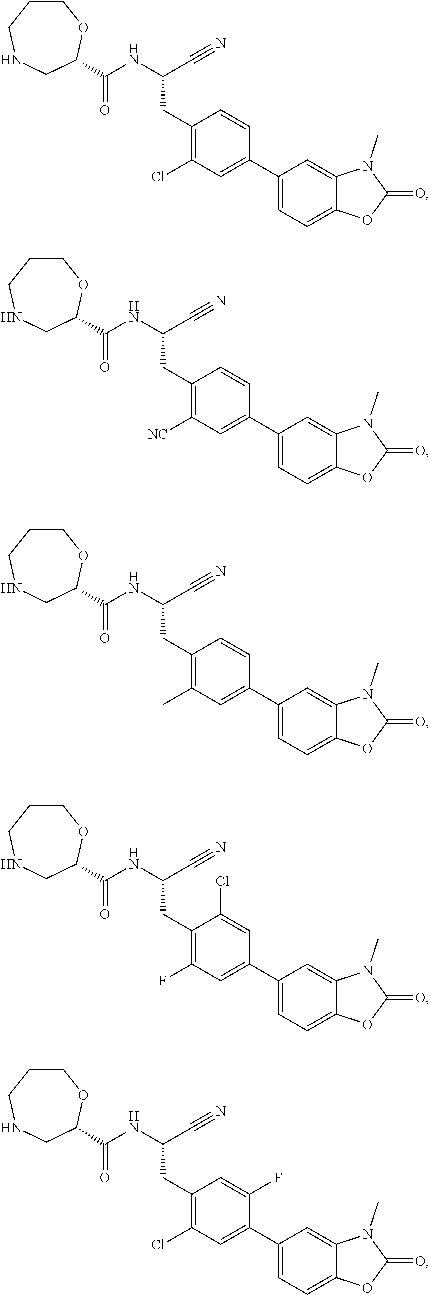 [SMILES: Cn1c(=O)oc2ccc(-c3ccc(C[C@@H](C#N)NC(=O)[C@@H]4CNCCCO4)c(Cl)c3)cc21 ; Cn1c(=O)oc2ccc(-c3ccc(C[C@@H](C#N)NC(=O)[C@@H]4CNCCCO4)c(C#N)c3)cc21 ; Cc1cc(-c2ccc3oc(=O)n(C)c3c2)ccc1C[C@@H](C#N)NC(=O)[C@@H]1CNCCCO1 ; Cn1c(=O)oc2ccc(-c3cc(F)c(C[C@@H](C#N)NC(=O)[C@@H]4CNCCCO4)c(Cl)c3)cc21 ; Cn1c(=O)oc2ccc(-c3cc(Cl)c(C[C@@H](C#N)NC(=O)[C@@H]4CNCCCO4)cc3F)cc21]

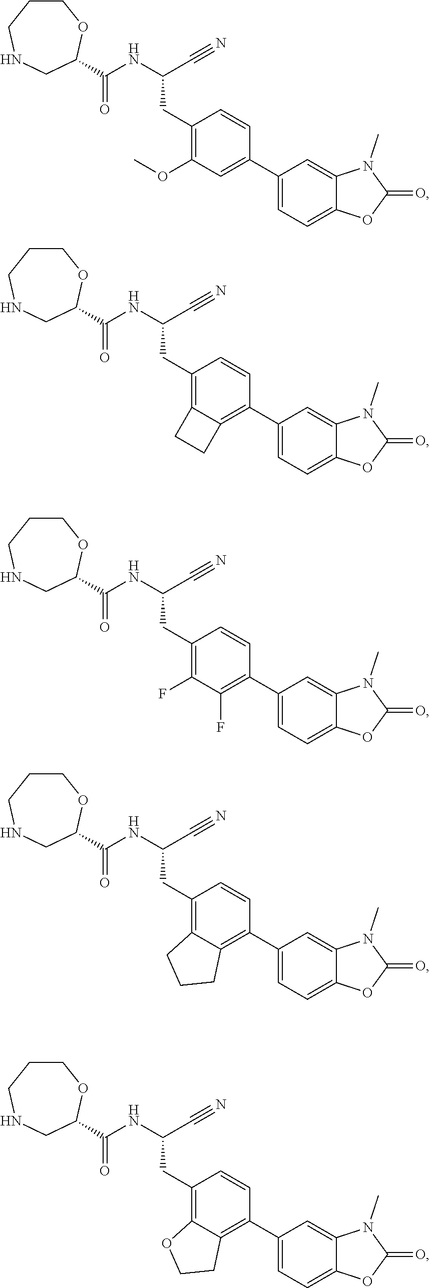 [SMILES: COc1cc(-c2ccc3oc(=O)n(C)c3c2)ccc1C[C@@H](C#N)NC(=O)[C@@H]1CNCCCO1 ; Cn1c(=O)oc2ccc(-c3ccc(C[C@@H](C#N)NC(=O)[C@@H]4CNCCCO4)c4c3CC4)cc21 ; Cn1c(=O)oc2ccc(-c3ccc(C[C@@H](C#N)NC(=O)[C@@H]4CNCCCO4)c(F)c3F)cc21 ; Cn1c(=O)oc2ccc(-c3ccc(C[C@@H](C#N)NC(=O)[C@@H]4CNCCCO4)c4c3CCC4)cc21 ; Cn1c(=O)oc2ccc(-c3ccc(C[C@@H](C#N)NC(=O)[C@@H]4CNCCCO4)c4c3CCO4)cc21]

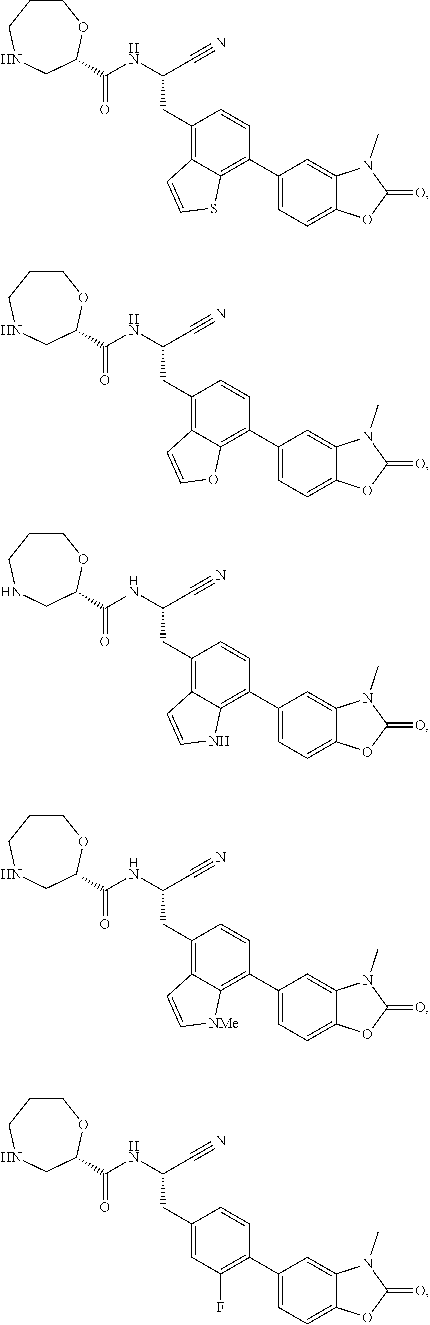 [SMILES: Cn1c(=O)oc2ccc(-c3ccc(C[C@@H](C#N)NC(=O)[C@@H]4CNCCCO4)c4ccsc34)cc21 ; Cn1c(=O)oc2ccc(-c3ccc(C[C@@H](C#N)NC(=O)[C@@H]4CNCCCO4)c4ccoc34)cc21 ; Cn1c(=O)oc2ccc(-c3ccc(C[C@@H](C#N)NC(=O)[C@@H]4CNCCCO4)c4cc[nH]c34)cc21 ; Cn1ccc2c(C[C@@H](C#N)NC(=O)[C@@H]3CNCCCO3)ccc(-c3ccc4oc(=O)n(C)c4c3)c21 ; Cn1c(=O)oc2ccc(-c3ccc(C[C@@H](C#N)NC(=O)[C@@H]4CNCCCO4)cc3F)cc21]

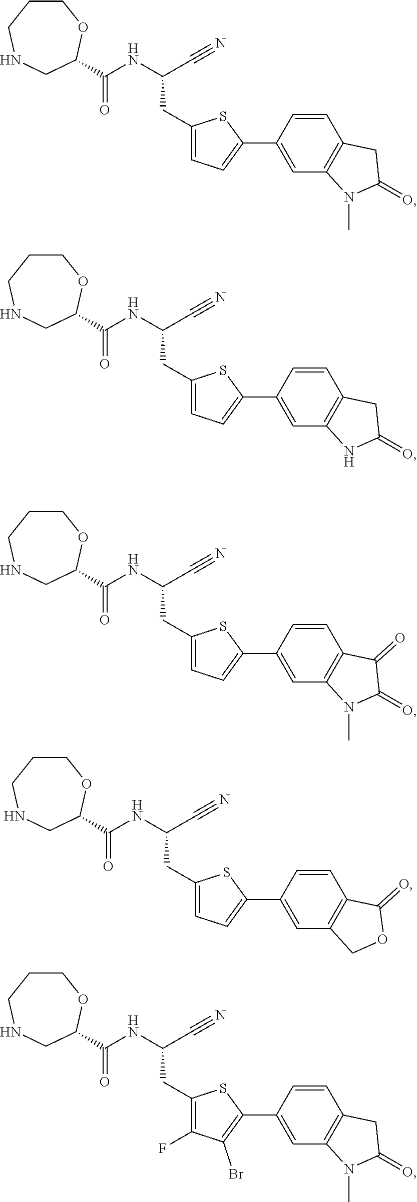 [SMILES: CN1C(=O)Cc2ccc(-c3ccc(C[C@@H](C#N)NC(=O)[C@@H]4CNCCCO4)s3)cc21 ; N#C[C@H](Cc1ccc(-c2ccc3c(c2)NC(=O)C3)s1)NC(=O)[C@@H]1CNCCCO1 ; CN1C(=O)C(=O)c2ccc(-c3ccc(C[C@@H](C#N)NC(=O)[C@@H]4CNCCCO4)s3)cc21 ; N#C[C@H](Cc1ccc(-c2ccc3c(c2)COC3=O)s1)NC(=O)[C@@H]1CNCCCO1 ; CN1C(=O)Cc2ccc(-c3sc(C[C@@H](C#N)NC(=O)[C@@H]4CNCCCO4)c(F)c3Br)cc21]

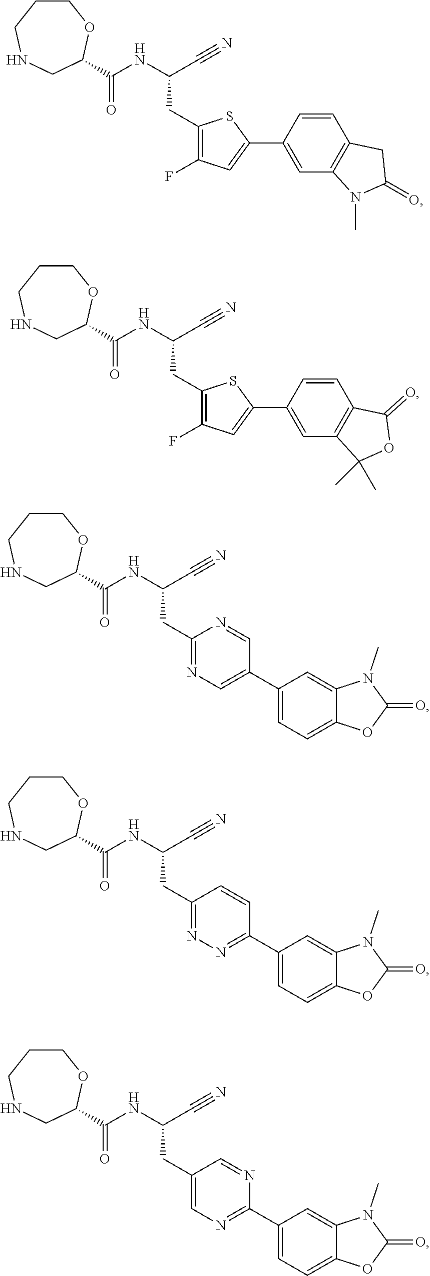 [SMILES: CN1C(=O)Cc2ccc(-c3cc(F)c(C[C@@H](C#N)NC(=O)[C@@H]4CNCCCO4)s3)cc21 ; CC1(C)OC(=O)c2ccc(-c3cc(F)c(C[C@@H](C#N)NC(=O)[C@@H]4CNCCCO4)s3)cc21 ; Cn1c(=O)oc2ccc(-c3cnc(C[C@@H](C#N)NC(=O)[C@@H]4CNCCCO4)nc3)cc21 ; Cn1c(=O)oc2ccc(-c3ccc(C[C@@H](C#N)NC(=O)[C@@H]4CNCCCO4)nn3)cc21 ; Cn1c(=O)oc2ccc(-c3ncc(C[C@@H](C#N)NC(=O)[C@@H]4CNCCCO4)cn3)cc21]

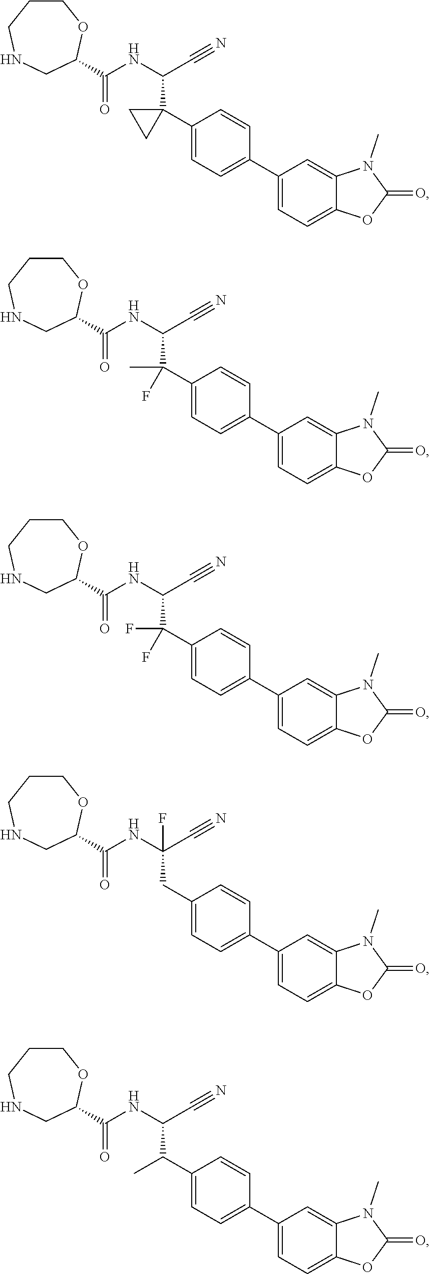 [SMILES: Cn1c(=O)oc2ccc(-c3ccc(C4([C@@H](C#N)NC(=O)[C@@H]5CNCCCO5)CC4)cc3)cc21 ; Cn1c(=O)oc2ccc(-c3ccc(C(C)(F)[C@@H](C#N)NC(=O)[C@@H]4CNCCCO4)cc3)cc21 ; Cn1c(=O)oc2ccc(-c3ccc(C(F)(F)[C@@H](C#N)NC(=O)[C@@H]4CNCCCO4)cc3)cc21 ; Cn1c(=O)oc2ccc(-c3ccc(C[C@](F)(C#N)NC(=O)[C@@H]4CNCCCO4)cc3)cc21 ; CC(c1ccc(-c2ccc3oc(=O)n(C)c3c2)cc1)[C@@H](C#N)NC(=O)[C@@H]1CNCCCO1]

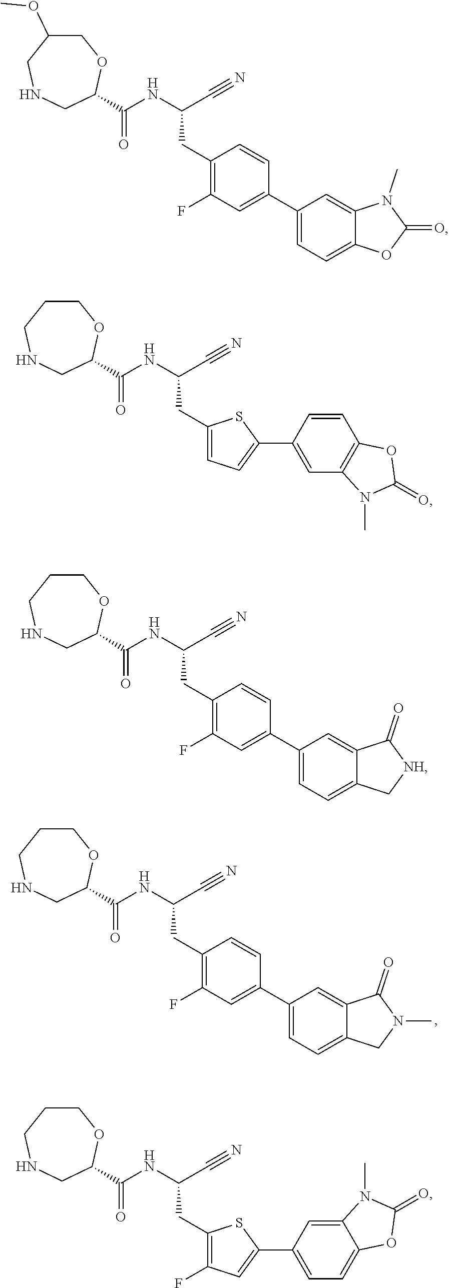 [SMILES: COC1CNC[C@@H](C(=O)N[C@H](C#N)Cc2ccc(-c3ccc4oc(=O)n(C)c4c3)cc2F)OC1 ; Cn1c(=O)oc2ccc(-c3ccc(C[C@@H](C#N)NC(=O)[C@@H]4CNCCCO4)s3)cc21 ; N#C[C@H](Cc1ccc(-c2ccc3c(c2)C(=O)NC3)cc1F)NC(=O)[C@@H]1CNCCCO1 ; CN1Cc2ccc(-c3ccc(C[C@@H](C#N)NC(=O)[C@@H]4CNCCCO4)c(F)c3)cc2C1=O ; Cn1c(=O)oc2ccc(-c3cc(F)c(C[C@@H](C#N)NC(=O)[C@@H]4CNCCCO4)s3)cc21]

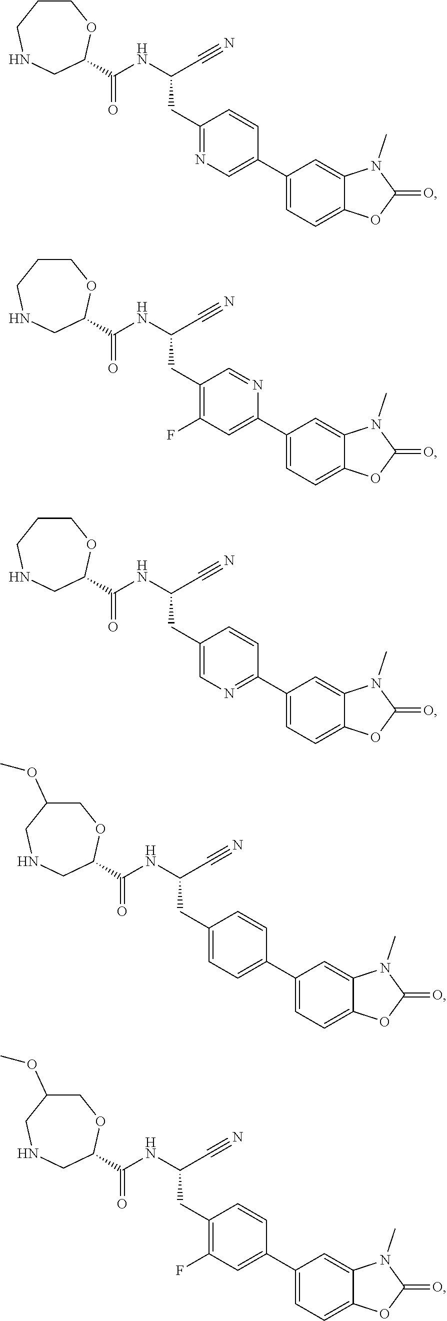 [SMILES: Cn1c(=O)oc2ccc(-c3ccc(C[C@@H](C#N)NC(=O)[C@@H]4CNCCCO4)nc3)cc21 ; Cn1c(=O)oc2ccc(-c3cc(F)c(C[C@@H](C#N)NC(=O)[C@@H]4CNCCCO4)cn3)cc21 ; Cn1c(=O)oc2ccc(-c3ccc(C[C@@H](C#N)NC(=O)[C@@H]4CNCCCO4)cn3)cc21 ; COC1CNC[C@@H](C(=O)N[C@H](C#N)Cc2ccc(-c3ccc4oc(=O)n(C)c4c3)cc2)OC1 ; COC1CNC[C@@H](C(=O)N[C@H](C#N)Cc2ccc(-c3ccc4oc(=O)n(C)c4c3)cc2F)OC1]

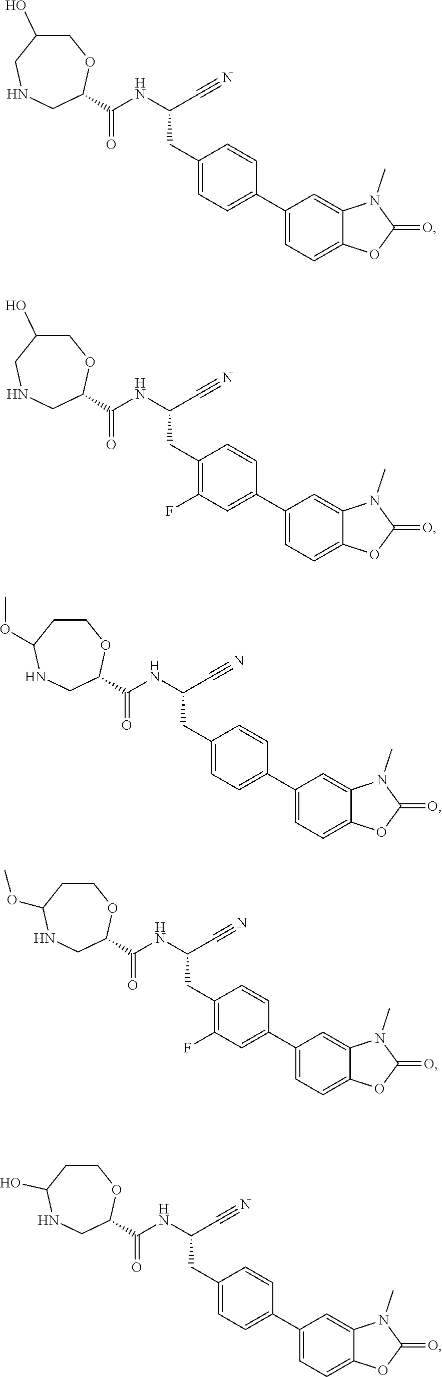 [SMILES: Cn1c(=O)oc2ccc(-c3ccc(C[C@@H](C#N)NC(=O)[C@@H]4CNCC(O)CO4)cc3)cc21 ; Cn1c(=O)oc2ccc(-c3ccc(C[C@@H](C#N)NC(=O)[C@@H]4CNCC(O)CO4)c(F)c3)cc21 ; COC1CCO[C@H](C(=O)N[C@H](C#N)Cc2ccc(-c3ccc4oc(=O)n(C)c4c3)cc2)CN1 ; COC1CCO[C@H](C(=O)N[C@H](C#N)Cc2ccc(-c3ccc4oc(=O)n(C)c4c3)cc2F)CN1 ; Cn1c(=O)oc2ccc(-c3ccc(C[C@@H](C#N)NC(=O)[C@@H]4CNC(O)CCO4)cc3)cc21]

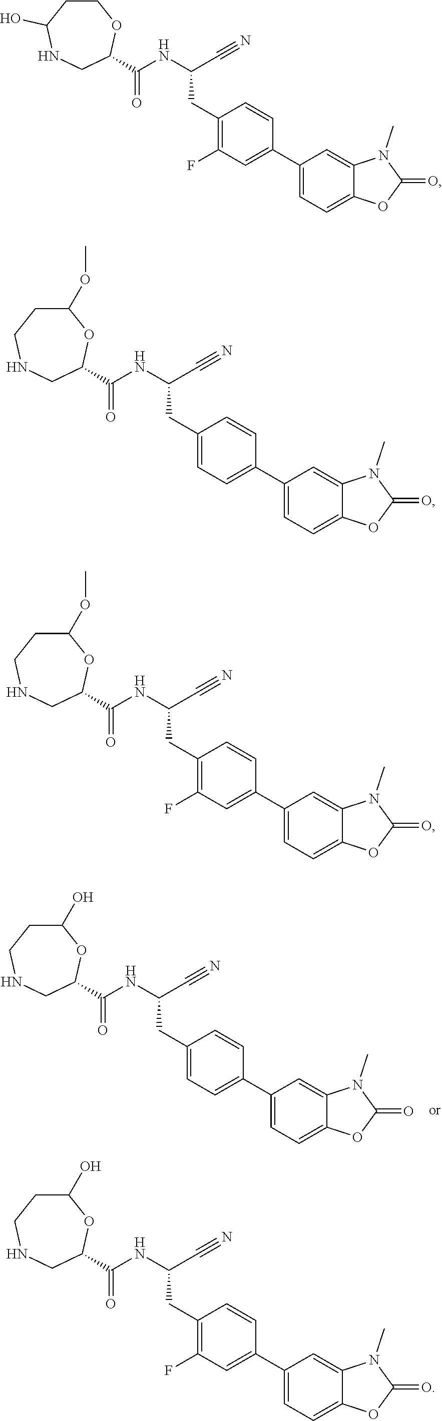 [SMILES: Cn1c(=O)oc2ccc(-c3ccc(C[C@@H](C#N)NC(=O)[C@@H]4CNC(O)CCO4)c(F)c3)cc21 ; COC1CCNC[C@@H](C(=O)N[C@H](C#N)Cc2ccc(-c3ccc4oc(=O)n(C)c4c3)cc2)O1 ; COC1CCNC[C@@H](C(=O)N[C@H](C#N)Cc2ccc(-c3ccc4oc(=O)n(C)c4c3)cc2F)O1 ; Cn1c(=O)oc2ccc(-c3ccc(C[C@@H](C#N)NC(=O)[C@@H]4CNCCC(O)O4)cc3)cc21 ; Cn1c(=O)oc2ccc(-c3ccc(C[C@@H](C#N)NC(=O)[C@@H]4CNCCC(O)O4)c(F)c3)cc21]

In one embodiment, the compound for use in one of the methods provided herein, or a pharmaceutically acceptable salt thereof, is selected from one of the following:

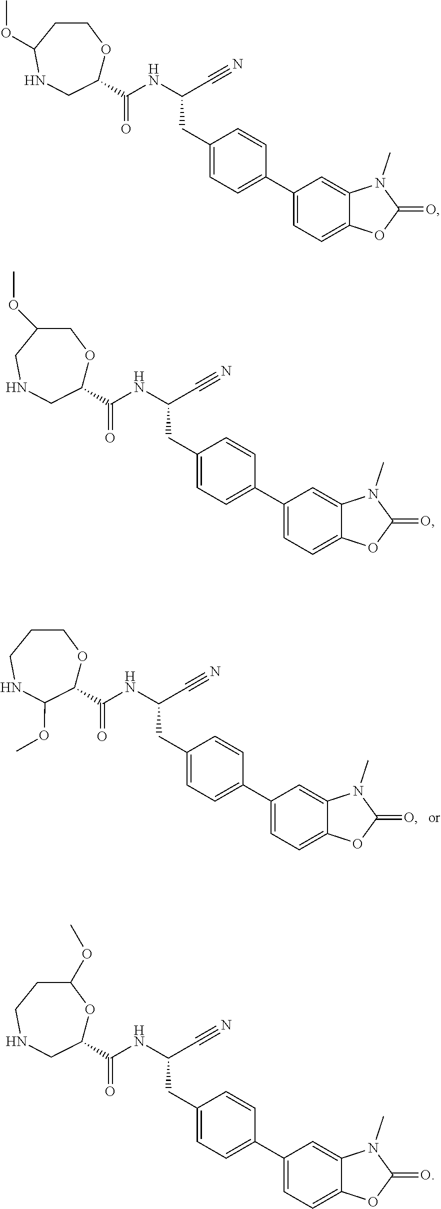 [SMILES: COC1CCO[C@H](C(=O)N[C@H](C#N)Cc2ccc(-c3ccc4oc(=O)n(C)c4c3)cc2)CN1 ; COC1CNC[C@@H](C(=O)N[C@H](C#N)Cc2ccc(-c3ccc4oc(=O)n(C)c4c3)cc2)OC1 ; COC1NCCCO[C@@H]1C(=O)N[C@H](C#N)Cc1ccc(-c2ccc3oc(=O)n(C)c3c2)cc1 ; COC1CCNC[C@@H](C(=O)N[C@H](C#N)Cc2ccc(-c3ccc4oc(=O)n(C)c4c3)cc2)O1]

In one embodiment, the compound for use in one of the methods provided herein, or a pharmaceutically acceptable salt thereof, is selected from one of the following:

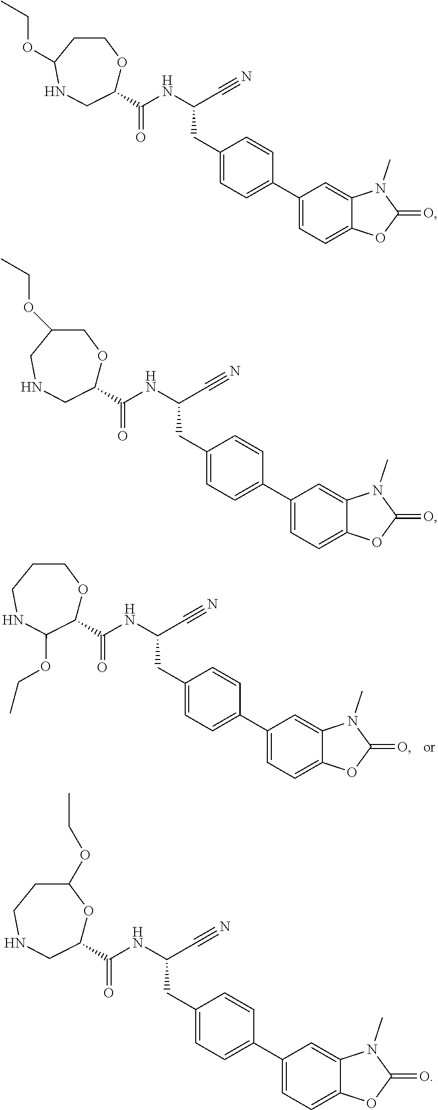 [SMILES: CCOC1CCO[C@H](C(=O)N[C@H](C#N)Cc2ccc(-c3ccc4oc(=O)n(C)c4c3)cc2)CN1 ; CCOC1CNC[C@@H](C(=O)N[C@H](C#N)Cc2ccc(-c3ccc4oc(=O)n(C)c4c3)cc2)OC1 ; CCOC1NCCCO[C@@H]1C(=O)N[C@H](C#N)Cc1ccc(-c2ccc3oc(=O)n(C)c3c2)cc1 ; CCOC1CCNC[C@@H](C(=O)N[C@H](C#N)Cc2ccc(-c3ccc4oc(=O)n(C)c4c3)cc2)O1]

In yet another embodiment, the compound for use in one of the methods provided herein, or a pharmaceutically acceptable salt thereof, is selected from one of the following:

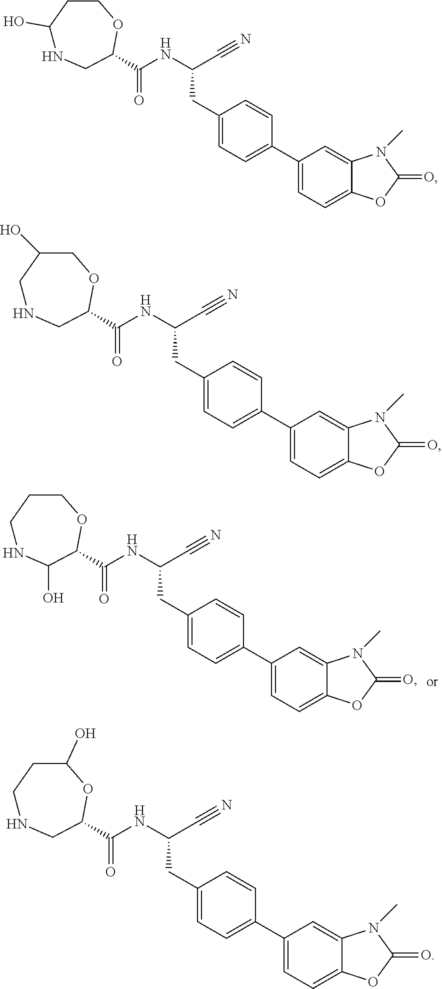 [SMILES: Cn1c(=O)oc2ccc(-c3ccc(C[C@@H](C#N)NC(=O)[C@@H]4CNC(O)CCO4)cc3)cc21 ; Cn1c(=O)oc2ccc(-c3ccc(C[C@@H](C#N)NC(=O)[C@@H]4CNCC(O)CO4)cc3)cc21 ; Cn1c(=O)oc2ccc(-c3ccc(C[C@@H](C#N)NC(=O)[C@H]4OCCCNC4O)cc3)cc21 ; Cn1c(=O)oc2ccc(-c3ccc(C[C@@H](C#N)NC(=O)[C@@H]4CNCCC(O)O4)cc3)cc21]

In a preferred embodiment, the compound for used in one of the methods provided herein, or a pharmaceutically acceptable salt thereof, is

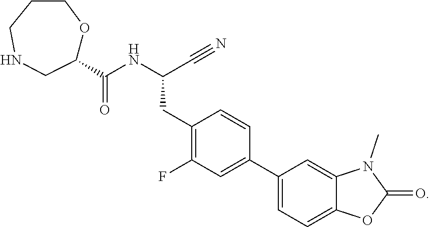 [SMILES: Cn1c(=O)oc2ccc(-c3ccc(C[C@@H](C#N)NC(=O)[C@@H]4CNCCCO4)c(F)c3)cc21]

In one embodiment, the compound for use in one of the methods provided herein, or a pharmaceutically acceptable salt thereof, is selected from one of the following:

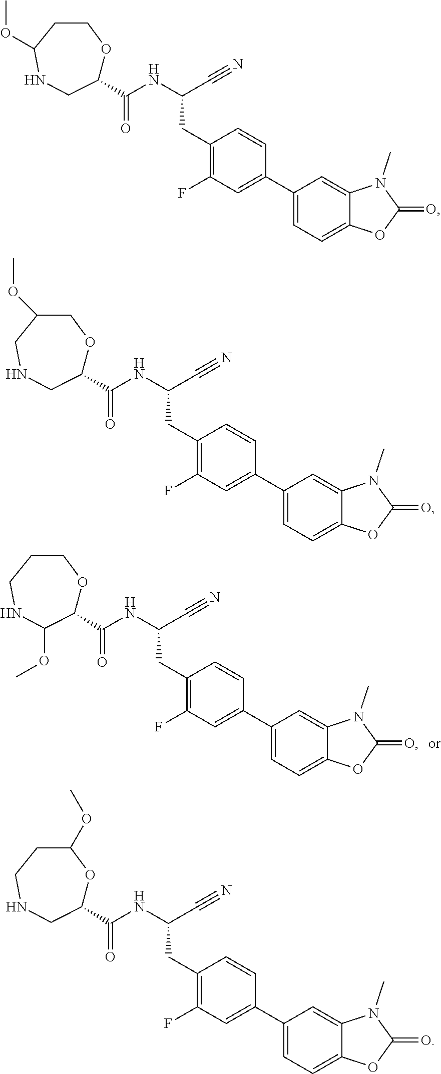 [SMILES: COC1CCO[C@H](C(=O)N[C@H](C#N)Cc2ccc(-c3ccc4oc(=O)n(C)c4c3)cc2F)CN1 ; COC1CNC[C@@H](C(=O)N[C@H](C#N)Cc2ccc(-c3ccc4oc(=O)n(C)c4c3)cc2F)OC1 ; COC1NCCCO[C@@H]1C(=O)N[C@H](C#N)Cc1ccc(-c2ccc3oc(=O)n(C)c3c2)cc1F ; COC1CCNC[C@@H](C(=O)N[C@H](C#N)Cc2ccc(-c3ccc4oc(=O)n(C)c4c3)cc2F)O1]

In one embodiment, the compound for use in one of the methods provided herein, or a pharmaceutically acceptable salt thereof, is selected from one of the following:

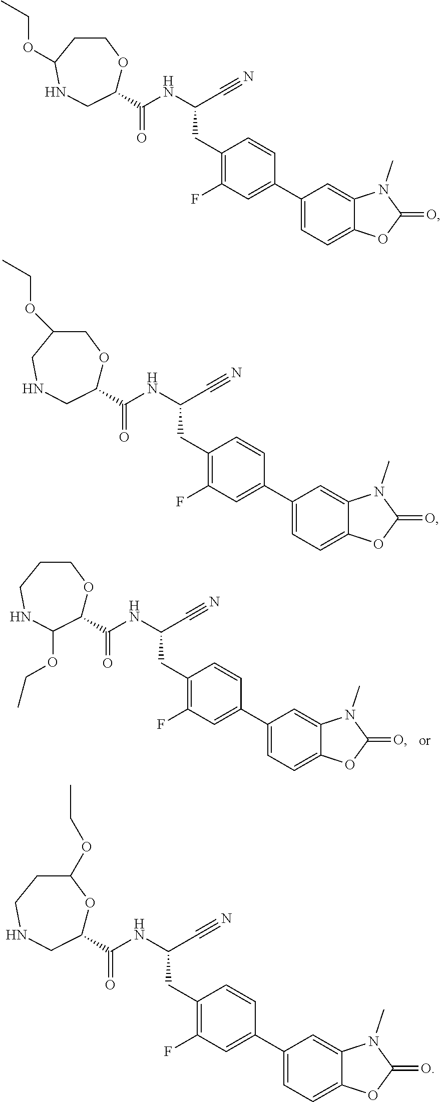 [SMILES: CCOC1CCO[C@H](C(=O)N[C@H](C#N)Cc2ccc(-c3ccc4oc(=O)n(C)c4c3)cc2F)CN1 ; CCOC1CNC[C@@H](C(=O)N[C@H](C#N)Cc2ccc(-c3ccc4oc(=O)n(C)c4c3)cc2F)OC1 ; CCOC1NCCCO[C@@H]1C(=O)N[C@H](C#N)Cc1ccc(-c2ccc3oc(=O)n(C)c3c2)cc1F ; CCOC1CCNC[C@@H](C(=O)N[C@H](C#N)Cc2ccc(-c3ccc4oc(=O)n(C)c4c3)cc2F)O1]

In yet even another embodiment, the compound for use in one of the methods provided herein, or a pharmaceutically acceptable salt thereof, is selected from one of the following:

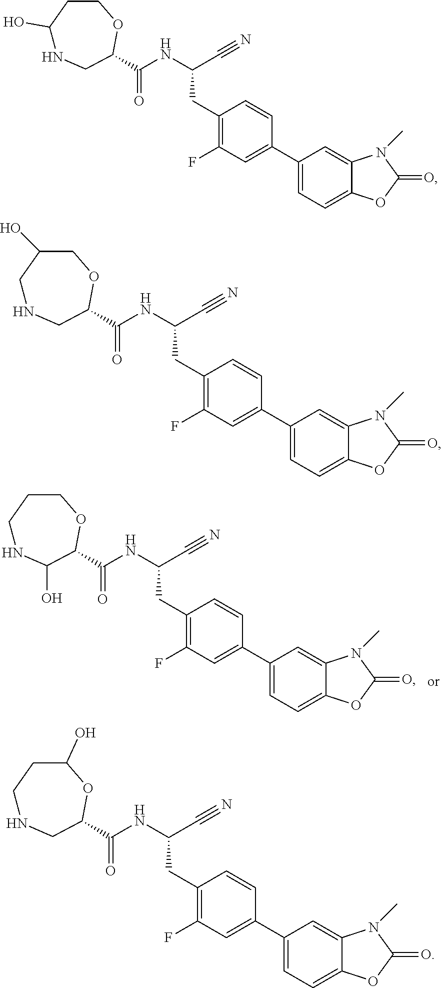 [SMILES: Cn1c(=O)oc2ccc(-c3ccc(C[C@@H](C#N)NC(=O)[C@@H]4CNC(O)CCO4)c(F)c3)cc21 ; Cn1c(=O)oc2ccc(-c3ccc(C[C@@H](C#N)NC(=O)[C@@H]4CNCC(O)CO4)c(F)c3)cc21 ; Cn1c(=O)oc2ccc(-c3ccc(C[C@@H](C#N)NC(=O)[C@H]4OCCCNC4O)c(F)c3)cc21 ; Cn1c(=O)oc2ccc(-c3ccc(C[C@@H](C#N)NC(=O)[C@@H]4CNCCC(O)O4)c(F)c3)cc21]

The compounds of Formula (I), (Ia), (Ib), or pharmaceutically acceptable salts thereof, may be used on their own but will generally be administered in the form of a pharmaceutical composition in which the Formula (I), (Ia), (Ib) compound/salt (active ingredient) is in association with pharmaceutically acceptable adjuvant(s), diluents(s) or carrier(s). Conventional procedures for the selection and preparation of suitable pharmaceutical formulations are described in, for example, “Pharmaceuticals—The Science of Dosage Form Designs”, M. E. Aulton, Churchill Livingstone, 2^{nd} Ed. 2002.

In one aspect, this disclosure provides a method of treating a disorder associated with altered DPP1 activity in a subject in need of treatment, comprising administering an effective amount of a compound of formulae (I), or a pharmaceutically acceptable salt thereof, to the subject for an administration period: wherein
the compound of formulae (I) is

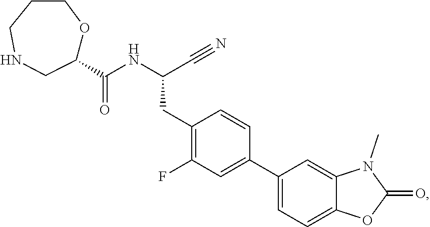 [SMILES: Cn1c(=O)oc2ccc(-c3ccc(C[C@@H](C#N)NC(=O)[C@@H]4CNCCCO4)c(F)c3)cc21]

and
the disorder associated with altered DPP1 activity is selected from rheumatoid arthritis, ulcerative colitis, chronic obstructive pulmonary disease (COPD), asthma, lupus nephritis, granulomatosis with polyangiitis (GPA), chronic rhinosinusitis with nasal polyps, chronic rhinosinusitis without nasyl polyps, hidradenitis suppurativa (HS) or neutrophilic asthma.

In one aspect, this disclosure provides a method of treating a disorder associated with altered DPP1 activity in a subject in need of treatment, comprising administering an effective amount of a compound of formulae (I), or a pharmaceutically acceptable salt thereof, to the subject for an administration period: wherein
the compound of formulae (I) is

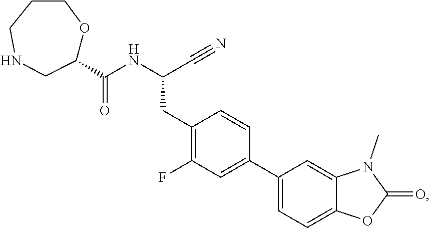 [SMILES: Cn1c(=O)oc2ccc(-c3ccc(C[C@@H](C#N)NC(=O)[C@@H]4CNCCCO4)c(F)c3)cc21]

and
the disorder associated with altered DPP1 activity is selected from rheumatoid arthritis, ulcerative colitis, lupus nephritis, granulomatosis with polyangiitis (GPA), chronic rhinosinusitis with nasal polyps, chronic rhinosinusitis without nasyl polyps), chronic rhinosinusitis with nasal polyps, chronic rhinosinusitis without nasyl polyps, hidradenitis suppurativa (HS) or neutrophilic asthma.

In embodiments, this present disclosure provides a compound selected from

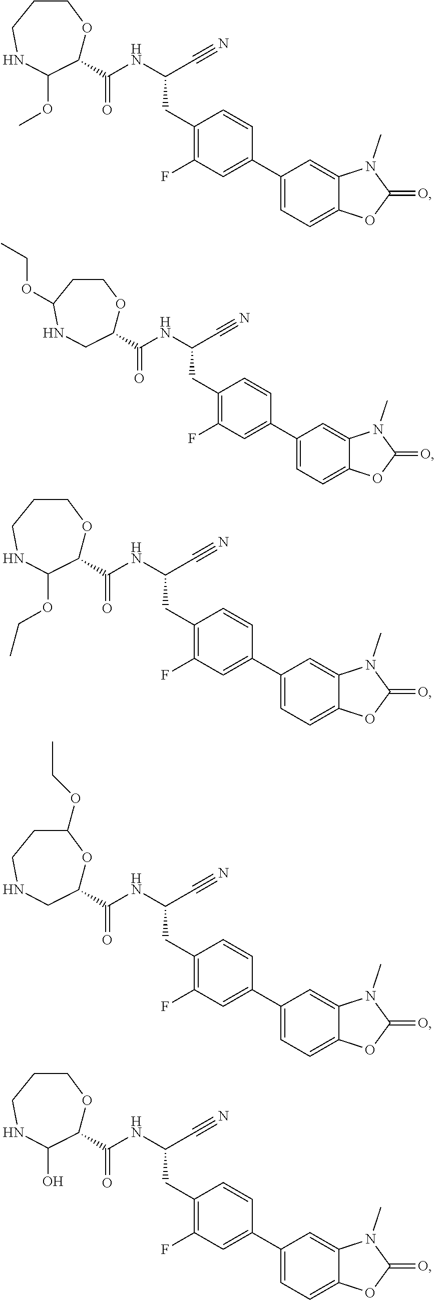 [SMILES: COC1NCCCO[C@@H]1C(=O)N[C@H](C#N)Cc1ccc(-c2ccc3oc(=O)n(C)c3c2)cc1F ; CCOC1CCO[C@H](C(=O)N[C@H](C#N)Cc2ccc(-c3ccc4oc(=O)n(C)c4c3)cc2F)CN1 ; CCOC1NCCCO[C@@H]1C(=O)N[C@H](C#N)Cc1ccc(-c2ccc3oc(=O)n(C)c3c2)cc1F ; CCOC1CCNC[C@@H](C(=O)N[C@H](C#N)Cc2ccc(-c3ccc4oc(=O)n(C)c4c3)cc2F)O1 ; Cn1c(=O)oc2ccc(-c3ccc(C[C@@H](C#N)NC(=O)[C@H]4OCCCNC4O)c(F)c3)cc21]

or a pharmaceutically acceptable salt or deuterated form thereof.

In embodiments, this present disclosure provides a compound having structure

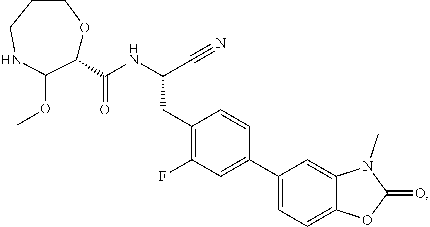 [SMILES: COC1NCCCO[C@@H]1C(=O)N[C@H](C#N)Cc1ccc(-c2ccc3oc(=O)n(C)c3c2)cc1F]

or a pharmaceutically acceptable salt or deuterated form thereof.

In embodiments, this present disclosure provides a compound having structure

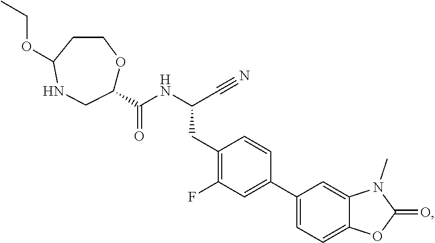 [SMILES: CCOC1CCO[C@H](C(=O)N[C@H](C#N)Cc2ccc(-c3ccc4oc(=O)n(C)c4c3)cc2F)CN1]

or a pharmaceutically acceptable salt or deuterated form thereof.

In embodiments, this present disclosure provides a compound having structure

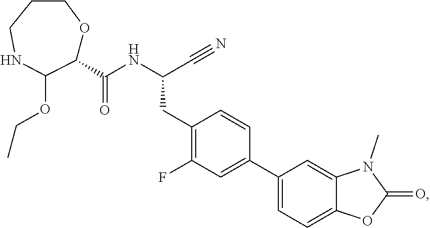 [SMILES: CCOC1NCCCO[C@@H]1C(=O)N[C@H](C#N)Cc1ccc(-c2ccc3oc(=O)n(C)c3c2)cc1F]

or a pharmaceutically acceptable salt or deuterated form thereof.

In embodiments, this present disclosure provides a compound having structure

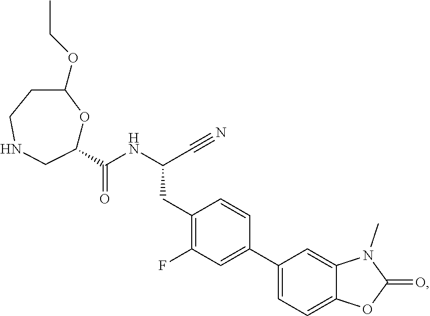 [SMILES: CCOC1CCNC[C@@H](C(=O)N[C@H](C#N)Cc2ccc(-c3ccc4oc(=O)n(C)c4c3)cc2F)O1]

or a pharmaceutically acceptable salt or deuterated form thereof.

In embodiments, this present disclosure provides a compound having structure

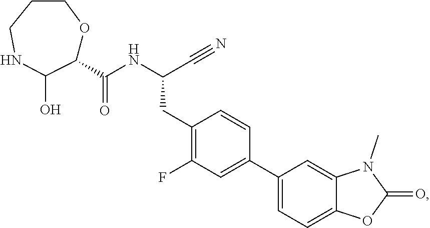 [SMILES: Cn1c(=O)oc2ccc(-c3ccc(C[C@@H](C#N)NC(=O)[C@H]4OCCCNC4O)c(F)c3)cc21]

or a pharmaceutically acceptable salt or deuterated form thereof.

In embodiments, the present disclosure provides a pharmaceutical composition(s) comprising an effective amount of a compound of Formula (I), (Ia), (Ib), or a pharmaceutically acceptable salt thereof, as hereinbefore defined in association with pharmaceutically acceptable adjuvant(s), diluent(s) or carrier(s).

The disclosure further provides a process for the preparation of a pharmaceutical composition of the disclosure which comprises mixing a compound of Formula (I), (Ia), (Ib), or a pharmaceutically acceptable salt thereof, as hereinbefore defined with a pharmaceutically acceptable adjuvant(s), diluents(s) or carrier(s).

The pharmaceutical compositions may be administered topically (e.g., to the skin or to the lung and/or airways) in the form, e.g., of creams, solutions, suspensions, heptafluoroalkane (HFA) aerosols and dry powder formulations, for example, formulations in the inhaler device known as the Turbuhaler®; or systemically, e.g., by oral administration in the form of tablets, capsules, syrups, powders or granules; or by parenteral administration in the form of a sterile solution, suspension or emulsion for injection (including intravenous, subcutaneous, intramuscular, intravascular or infusion); or by rectal administration in the form of suppositories.

For oral administration the compound of the disclosure may be admixed with adjuvant(s), diluent(s) or carrier(s), for example, lactose, saccharose, sorbitol, mannitol; starch, for example, potato starch, corn starch or amylopectin; cellulose derivative; binder, for example, gelatine or polyvinylpyrrolidone; disintegrant, for example cellulose derivative, and/or lubricant, for example, magnesium stearate, calcium stearate, polyethylene glycol, wax, paraffin, and the like, and then compressed into tablets. If coated tablets are required, the cores, prepared as described above, may be coated with a suitable polymer dissolved or dispersed in water or readily volatile organic solvent(s). Alternatively, the tablet may be coated with a concentrated sugar solution which may contain, for example, gum arabic, gelatine, talcum and titanium dioxide.

For the preparation of soft gelatine capsules, the compound of the disclosure may be admixed with, for example, a vegetable oil or polyethylene glycol. Hard gelatine capsules may contain granules of the compound using pharmaceutical excipients like the abovementioned excipients for tablets. Additionally, liquid or semisolid formulations of the compound of the disclosure may be filled into hard gelatine capsules.

Liquid preparations for oral application may be in the form of syrups, solutions or suspensions. Solutions, for example may contain the compound of the disclosure, the balance being sugar and a mixture of ethanol, water, glycerol and propylene glycol. Optionally such liquid preparations may contain coloring agents, flavoring agents, saccharine and/or carboxymethylcellulose as a thickening agent. Furthermore, other excipients known to those skilled in art may be used when making formulations for oral use.

The dosage administered will vary with the compound of Formula (I), (Ia), (Ib), employed, the mode of administration, and the treatment outcome desired. For example, in one embodiment, the daily dosage of the compound of Formula (I), (Ia), (Ib), if inhaled, may be in the range from 0.05 micrograms per kilogram body weight (μg/kg) to 100 micrograms per kilogram body weight (μg/kg). Alternatively, in one embodiment, if the composition comprising a compound of Formula (I), (Ia), (Ib), is administered orally, then the daily dosage of the compound of the disclosure may be in the range from 0.01 micrograms per kilogram body weight (μg/kg) to 100 milligrams per kilogram body weight (mg/kg).

The compounds of Formula (I), (Ia), (Ib), or pharmaceutically acceptable salts thereof, may be used on their own but will generally be administered in the form of a pharmaceutical composition in which the Formula (I), (Ia), (Ib) compound/salt (active ingredient) is in association with pharmaceutically acceptable adjuvant(s), diluents(s) or carrier(s). Conventional procedures for the selection and preparation of suitable pharmaceutical formulations are described in, for example, “Pharmaceuticals—The Science of Dosage Form Designs”, M. E. Aulton, Churchill Livingstone, 2^{nd} Ed. 2002.

Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

For example, in one embodiment, a compound of the present invention is administered to a patient in a method for treating an obstructive disease of the airway. The obstructive disease of the airway, in one embodiment, is asthma (e.g., bronchial, allergic, intrinsic, extrinsic, exercise-induced, drug-induced (including aspirin and NSAID-induced and dust-induced asthma, both intermittent and persistent and of all severities) airway hyper-responsiveness, chronic obstructive pulmonary disease (COPD), bronchitis (e.g., infectious bronchitis, eosinophilic bronchitis), emphysema, cystic fibrosis (CF), bronchiectasis (e.g., non-CF bronchiectasis (NCFBE) and bronchiectasis associated with CF), cystic fibrosis; sarcoidosis; alpha-1 antitrypsin (A1AT) deficiency, farmer's lung and related diseases, hypersensitivity pneumonitis, lung fibrosis (including idiopathic pulmonary fibrosis, cryptogenic fibrosing alveolitis, idiopathic interstitial pneumonias, fibrosis complicating anti-neoplastic therapy and chronic infection, including tuberculosis and aspergillosis and other fungal infections), complications of lung transplantation, vasculitic and thrombotic disorders of the lung vasculature, pulmonary hypertension (e.g., pulmonary arterial hypertension), antitussive activity including treatment of chronic cough associated with inflammatory and secretory conditions of the airways, iatrogenic cough, acute and chronic rhinitis including rhinitis medicamentosa, and vasomotor rhinitis; perennial and seasonal allergic rhinitis including rhinitis nervosa (hay fever), nasal polyposis; acute viral infection including the common cold, and infection due to a respiratory virus (e.g., respiratory syncytial virus, influenza, coronavirus (including SARS) and adenovirus), acute lung injury, acute respiratory distress syndrome (ARDS), as well as exacerbations of each of the foregoing respiratory tract disease states.

In one embodiment, the method of treatment provided herein are used to treat cystic fibrosis (CF). CF is caused by abnormalities in the CF transmembrane conductance regulator protein, causing chronic lung infections (particularly with Pseudomonas aeruginosa) and excessive inflammation, and leading to bronchiectasis, declining lung function, respiratory insufficiency and quality of life. The inflammatory process is dominated by neutrophils that produce NE, as well as other destructive NSPs including CatG and PR3, that directly act upon extracellular matrix proteins and play a role in the host response to inflammation and infection (Dittrich et al., Eur Respir J. 2018; 51 (3)). The methods provided herein employ reversible inhibitors of DPP1. Without wishing to be bound by theory, it is thought that the compounds of Formula (I), (Ia), (Ib), administered via the methods provided herein have beneficial effects via inhibiting the activation of NSPs and decreasing inflammation, which in turn leads to a decrease in pulmonary exacerbations, a decrease in the rate of pulmonary exacerbations, and/or an improvement in lung function (e.g., forced expiratory volume in 1 second [FEV₁]) in CF patients.

In one embodiment, a method is provided for treating CF comprising administering to a CF patient in need of treatment, a composition comprising an effective amount of a compound of Formula (I), (Ia), (Ib) or a pharmaceutically acceptable salt thereof. Administration routes include oral administration. Administration schedules and administration periods can be determined by the user of the method, e.g., a prescribing physician. In one embodiment, administration is once daily. In another embodiment, administration is twice daily. In another embodiment, administration is every other day, every third day, 3× per week or 4× per week.

In one CF treatment method, a composition comprising an effective amount of a compound of Formula (I), (Ia), (Ib), or a pharmaceutically acceptable salt thereof, is administered to a CF patient in need of treatment for an administration period. The method comprises improving the lung function of the patient during the administration period, as compared to the lung function of the patient prior to the administration period. In a further embodiment, the compound is administered orally, once daily. The improvement in lung function in one embodiment, is measured by spirometry.

Improving the lung function of the patient, in one embodiment, comprises increasing the patient's forced expiratory volume in 1 second (FEV₁), increasing the patient's forced vital capacity (FVC), increasing the patient's peak expiratory flow rate (PEFR), or increasing the patient's forced expiratory flow between 25% and 75% of FVC (FEF_{(25-75 %)}), as compared to the respective value prior to the administration period. Increasing, in one embodiment, is by about 5%, by about 10%, by about 15%, by about 20%, by about 25%, by about 30%, by about 35%, by about 40%, by about 45% or by about 50% of the respective value. Increasing, in one embodiment, is by at least about 5%, by at least about 10%, by at least about 15%, by at least about 20%, by at least about 25%, by at least about 30%, by at least about 35%, by at least about 40%, by at least about 45% or by at least about 50%. In yet another embodiment, the increasing is by about 5% to about 50%, by about 5% to about 40%, by about 5% to about 30% or by about 5% to about 20%. In even another embodiment, increasing is by about 10% to about 50%, by about 15% to about 50%, by about 20% to about 50%, or by about 25% to about 50%.

In one embodiment of a method provided herein, a composition comprising an effective amount of a compound of Formula (I), (Ia), (Ib), or a pharmaceutically acceptable salt thereof, is administered to a bronchiectasis patient in need of treatment for an administration period. Bronchiectasis is considered a pathological endpoint that results from many disease processes and is a persistent or progressive condition characterized by dilated thick-walled bronchi. The symptoms vary from intermittent episodes of expectoration and infection localized to the region of the lung that is affected to persistent daily expectoration often of large volumes of purulent sputum. Bronchiectasis may be associated with other non-specific respiratory symptoms. The underlying pathological process of bronchiectasis, without wishing to be bound by theory, has been reported as damage to the airways which results from an event or series of events where inflammation is central to the process (Guideline for non-CF Bronchiectasis, Thorax, July 2010, V. 65 (Suppl 1), incorporated by reference herein in its entirety for all purposes).

Bronchiectasis is considered a pathological endpoint that results from many disease processes and is a persistent or progressive condition characterized by dilated thick-walled bronchi. The symptoms vary from intermittent episodes of expectoration and infection localized to the region of the lung that is affected to persistent daily expectoration often of large volumes of purulent sputum. Bronchiectasis may be associated with other non-specific respiratory symptoms. The underlying pathological process of bronchiectasis, without wishing to be bound by theory, has been reported as damage to the airways which results from an event or series of events where inflammation is central to the process (Guideline for non-CF Bronchiectasis, Thorax, July 2010, V. 65 (Suppl 1), incorporated by reference herein in its entirety for all purposes).

The methods provided herein employ reversible inhibitors of DPP1. Without wishing to be bound by theory, it is thought that the compounds of Formula (I), (Ia), (Ib), administered via the methods provided herein have beneficial effects via decreasing inflammation and mucus hypersecretion, which in some embodiments, leads to a decrease in pulmonary exacerbations, a decrease in the rate of pulmonary exacerbations, and/or an improvement in lung function (cough, sputum production, and forced expiratory volume in 1 second [FEV₁]) in bronchiectasis patients. Without wishing to be bound by theory, it is thought that the methods provided herein modify bronchiectasis progression by reducing the accelerated rate of lung function decline or lung tissue destruction.

In one embodiment, the bronchiectasis is non-CF bronchiectasis.

In one embodiment, the method for treating bronchiectasis comprises improving lung function of the patient during the administration period, as compared to the lung function of the patient prior to the administration period.

A pulmonary exacerbation, in one embodiment, is characterized by three or more of the following symptoms exhibited for at least 48 hours by the patient: (1) increased cough; (2) increased sputum volume or change in sputum consistency; (3) increased sputum purulence; (4) increased breathlessness and/or decreased exercise tolerance; (5) fatigue and/or malaise; (6) hemoptysis. In a further embodiment, the three or more symptoms result in a physician's decision to prescribe an antibiotic(s) to the patient exhibiting the symptoms.

In one embodiment of a method for treating bronchiectasis, the method comprises decreasing the rate of pulmonary exacerbation in the subject, compared to the rate of pulmonary exacerbation experienced by the subject prior to the administration period of the composition, or compared to a control subject with bronchiectasis that is not subject to the method of treatment. In a further embodiment, the bronchiectasis is non-CF bronchiectasis.

In another aspect, a method for treating chronic rhinosinusitis (CRS) in a subject in need thereof is provided. The method comprises in one embodiment, administering to the subject for an administration period, a pharmaceutical composition comprising an effective amount of a compound of Formula (I), (Ia), (Ib), or a pharmaceutically acceptable salt thereof.

The chronic rhinosinusitis is chronic rhinosinusitis without nasal polyps (CRSsNP), or chronic rhinosinusitis with nasal polyps (CRSwNP). In some embodiments, the chronic rhinosinusitis is chronic rhinosinusitis without nasal polyps (CRSsNP). In some embodiments, the chronic rhinosinusitis is chronic rhinosinusitis with nasal polyps (CRSwNP). In some embodiments, the chronic rhinosinusitis is refractory chronic rhinosinusitis. In some embodiments, the refractory chronic rhinosinusitis is refractory chronic rhinosinusitis without nasal polyps (CRSsNP). In some embodiments, the refractory chronic rhinosinusitis is refractory chronic rhinosinusitis with nasal polyps (CRSwNP).

In some embodiments, the subject exhibits one or more symptoms of CRS. In some embodiments, the one or more symptoms of CRS are: (a) nasal congestion; (b) nasal obstruction; (c) nasal discharge; (d) post-nasal drip; (e) facial pressure; (f) facial pain; (g) facial fullness; (h) reduced smell; (i) depression; (j) mucosal edema; (k) mucopurulent discharge; (1) obstruction of the middle meatus; (m) mucosal changes within the ostiomeatal complex and sinuses; (n) rhinorrhea; or (o) any combinations thereof. In some embodiments, obstruction of the middle meatus is mucosal obstruction, edematous obstruction, or a combination thereof.

In some embodiments, the administration of the pharmaceutical composition reduces, diminishes the severity of, delays the onset of, or eliminates one or more symptoms of CRS. In some embodiments, the one or more symptoms of CRS are: (a) nasal congestion; (b) nasal obstruction; (c) nasal discharge; (d) post-nasal drip; (e) facial pressure; (f) facial pain; (g) facial fullness; (h) reduced smell; (i) depression; (j) mucosal edema; (k) mucopurulent discharge; (1) obstruction of the middle meatus; (m) mucosal changes within the ostiomeatal complex and sinuses; (n) rhinorrhea; (o) or any combinations thereof. In some embodiments, the administration of the pharmaceutical composition enhances sinus drainage.

In some embodiments, the methods comprise reducing a composite severity score of one or more symptoms of CRS. As used herein, the “composite severity score” is a quantitative measure of all the symptoms of CRS exhibited by the subject. In some embodiments, the composite severity score is a sum total of all the daily symptoms exhibited by the subject. In some embodiments, the composite severity score is reduced during or subsequent to the administration period, as compared to the composite severity score measured prior to the administration period. In some embodiments, the one or more symptoms of CRS exhibited by the subject may be any symptoms described herein or known in the art to be associated with CRS. In some embodiments, the one or more symptoms of CRS are: nasal congestion, reduced smell, rhinorrhea, or any combination thereof. In some embodiments, the rhinorrhea is anterior rhinorrhea. In some embodiments, the rhinorrhea is posterior rhinorrhea.

In some embodiments, the methods comprise decreasing the Sino-Nasal Outcome Test-₂₂ (SNOT-22) score of the subject during the administration period or subsequent to the administration period, compared to the SNOT-22 score of the subject prior to the administration period. As used herein, “SNOT-22” is a patient-reported measure of outcome developed for use in CRS with or without nasal polyps and contains 22 individual questions. The questions cover a broad range of health and health-related quality of life problems including physical problems, functional limitations and emotional consequences. The theoretical range of the SNOT-22 score is 0-110, with lower scores implying a better health-related quality of life. Further details of SNOT-22 are provided in Hopkins, et al., Clin. Otolaryngol. 2009, 34, 447-454, and Kennedy, et al., Ann Allergy Asthma Immunol. 2013 October; 111 (4): 246-251, the contents of which are incorporated herein by reference in its entirety.

Hidradenitis suppurativa (HS) is a chronic relapsing inflammatory disorder. The symptoms include skin lesions that are often associated hair follicles, and may be painful, inflamed and/or swollen. In some cases, when the skin lesions heal, they can recur, and may lead to tunnels under the skin and progressive scarring. Since HS is a chronic condition, it can persist for many years and also, worsen over time, with serious effects on quality of life, physochological and emotional well-being. In fact, HS pateints have increased rates of anxiety and depression with a risk of suicide two and a half times that of the general population.

HS patients are categorized according to disease severity, termed Hurley staging, as mild (Stage I), moderate (Stage II), or severe (Stage III). Although more than 200,000 cases of HS are diagnosed in the U.S. per year, this disease can be difficult to diagnose and requires specialized care. HS may be mistaken for an infection, an ingrown hair or other conditions. Moreover, current treatment options are limited and lack efficacy.

In one aspect, a method of treating HS in a subject in need thereof is provided. The method comprises in one embodiment, administering to the subject for an administration period, a pharmaceutical composition comprising an effective amount of a compound of Formula (I), (Ia), (Ib), or a pharmaceutically acceptable salt thereof. In a further embodiment, the method of treating HS comprises reducing neutrophilic inflammation in the subject.

The HS in one embodiment, is Hurley Stage I HS, Hurley Stage II HS or Hurley Stage III HS. In some embodiments, the HS is Hurley Stage I HS. In some embodiments, the HS is Hurley Stage II HS. In some embodiments, the HS is Hurley Stage III HS.

In some embodiments, the disorder mediated by dipeptidyl peptidase 1 (DPP1) is Granulomatosis with polyangiitis (GPA).

The disclosure provides methods of treating cancer in a subject in need thereof, comprising, administering to the subject, a pharmaceutical composition comprising an effective amount of any one of the compounds disclosed herein. The disclosure provides methods of treating cancer-induced pain in a subject having cancer, comprising, administering to the subject for an administration period, a pharmaceutical composition comprising an effective amount of any one of the compounds disclosed herein. In some embodiments, the cancer-induced pain is cancer-induced bone pain. The disclosure also provides methods of treating cancer-induced bone pain in a subject having cancer, comprising, administering to the subject for an administration period, a pharmaceutical composition comprising an effective amount of any one of the compounds disclosed herein.

In some embodiments, the cancer comprises a primary solid tumor. In some embodiments, the cancer is selected from the group consisting of bladder cancer, lung cancer, brain cancer, ovarian cancer, pancreatic cancer, colorectal cancer, prostate cancer, liver cancer, hepatocellular carcinoma, kidney cancer, stomach cancer, skin cancer, fibroid cancer, lymphoma, virus-induced cancer, oropharyngeal cancer, testicular cancer, thymus cancer, thyroid cancer, melanoma, and bone cancer.

In some embodiments, the cancer is bladder cancer. In some embodiments, the cancer is lung cancer. In some embodiments, the cancer is brain cancer. In some embodiments, the cancer is ovarian cancer. In some embodiments, the cancer is pancreatic cancer. In some embodiments, the cancer is colorectal cancer. In some embodiments, the cancer is prostate cancer. In some embodiments, the cancer is liver cancer. In some embodiments, the cancer is hepatocellular carcinoma. In some embodiments, the cancer is kidney cancer. In some embodiments, the cancer is stomach cancer. In some embodiments, the cancer is skin cancer. In some embodiments, the cancer is fibroid cancer. In some embodiments, the cancer is lymphoma. In some embodiments, the cancer is virus-induced cancer. In some embodiments, the cancer is oropharyngeal cancer. In some embodiments, the cancer is testicular cancer. In some embodiments, the cancer is thymus cancer. In some embodiments, the cancer is thyroid cancer. In some embodiments, the cancer is melanoma. In some embodiments, the cancer is bone cancer. In some embodiments, the fibroid cancer is leiomyosarcoma.

In some embodiments, the breast cancer comprises ductal carcinoma, lobular carcinoma, medullary carcinoma, colloid carcinoma, tubular carcinoma, or inflammatory breast cancer. In some embodiments, the breast cancer comprises ductal carcinoma. In some embodiments, the breast cancer comprises lobular carcinoma. In some embodiments, the breast cancer comprises medullary carcinoma. In some embodiments, the breast cancer comprises colloid carcinoma. In some embodiments, the breast cancer comprises tubular carcinoma. In some embodiments, the breast cancer comprises inflammatory breast cancer.

In some embodiments, the breast cancer is triple-negative breast cancer. In some embodiments, the breast cancer does not respond to hormonal therapy or therapeutics that target the HER2 protein receptors.

In some embodiments, the lymphoma is Hodgkin's lymphoma, non-Hodgkin's lymphoma, diffuse large B-cell lymphoma, B-cell immunoblastic lymphoma, Natural Killer cell lymphoma, T-cell lymphoma, Burkitt lymphoma or Kaposi's Sarcoma. In some embodiments, the lymphoma is Hodgkin's lymphoma. In some embodiments, the lymphoma is non-Hodgkin's lymphoma. In some embodiments, the lymphoma is diffuse large B-cell lymphoma. In some embodiments, the lymphoma is B-cell immunoblastic lymphoma. In some embodiments, the lymphoma is Natural Killer cell lymphoma. In some embodiments, the lymphoma is T-cell lymphoma. In some embodiments, the lymphoma is Burkitt lymphoma. In some embodiments, the lymphoma is Kaposi's Sarcoma.

In some embodiments, the brain cancer is astrocytoma, anaplastic astrocytoma, glioblastoma multiforme, oligodendroglioma, ependymoma, meningioma, schwannoma, or medulloblastoma. In some embodiments, the brain cancer is astrocytoma. In some embodiments, the brain cancer is anaplastic astrocytoma. In some embodiments, the brain cancer is glioblastoma multiforme. In some embodiments, the brain cancer is oligodendroglioma. In some embodiments, the brain cancer is ependymoma. In some embodiments, the brain cancer is meningioma. In some embodiments, the brain cancer is schwannoma. In some embodiments, the brain cancer is medulloblastoma.

In some embodiments, the cancer is liquid tumor. In some embodiments, the liquid tumor is selected from the group consisting of acute myeloid leukemia (AML), acute lymphoblastic leukemia, acute lymphocytic leukemia, acute promyelocytic leukemia, chronic myeloid leukemia, hairy cell leukemia, myeloproliferative disorders, Natural Killer cell leukemia, blastic plasmacytoid dendritic cell neoplasm, chronic myelogenous leukemia (CML), mastocytosis, chronic lymphocytic leukemia (CLL), multiple myeloma (MM), and myelodysplastic syndrome (MDS). In some embodiments, the liquid tumor is acute myeloid leukemia (AML). In some embodiments, the liquid tumor is acute lymphoblastic leukemia. In some embodiments, the liquid tumor is acute lymphocytic leukemia. In some embodiments, the liquid tumor is acute promyelocytic leukemia. In some embodiments, the liquid tumor is chronic myeloid leukemia. In some embodiments, the liquid tumor is hairy cell leukemia. In some embodiments, the liquid tumor is a myeloproliferative disorder. In some embodiments, the liquid tumor is Natural Killer cell leukemia. In some embodiments, the liquid tumor is blastic plasmacytoid dendritic cell neoplasm. In some embodiments, the liquid tumor is chronic myelogenous leukemia (CML). In some embodiments, the liquid tumor is mastocytosis. In some embodiments, the liquid tumor is chronic lymphocytic leukemia (CLL). In some embodiments, the liquid tumor is multiple myeloma (MM). In some embodiments, the liquid tumor is myelodysplastic syndrome (MDS).

In some embodiments, the cancer is a pediatric cancer. In some embodiments, the pediatric cancer is neuroblastoma, Wilms tumor, rhabdomyosarcoma, retinoblastoma, osteosarcoma or Ewing sarcoma. In some embodiments, the pediatric cancer is neuroblastoma. In some embodiments, the pediatric cancer is Wilms tumor. In some embodiments, the pediatric cancer is rhabdomyosarcoma. In some embodiments, the pediatric cancer is retinoblastoma. In some embodiments, the pediatric cancer is osteosarcoma. In some embodiments, the pediatric cancer is Ewing sarcoma.

In some embodiments, the cancer is metastatic cancer. In some embodiments, the subject is at a risk for developing metastatic cancer. In some embodiments, the metastatic cancer comprises metastasis of breast cancer to the brain, bone, pancreas, lymph nodes, and/or liver. In some embodiments, the metastatic cancer comprises metastasis of bone cancer to the lung. In some embodiments, the metastatic cancer comprises metastasis of colorectal cancer to the peritoneum, the pancreas, the stomach, the lung, the liver, the kidney, and/or the spleen. In some embodiments, the metastatic cancer comprises metastasis of stomach cancer to the mesentery, the spleen, the pancreas, the lung, the liver, the adrenal gland, and/or the ovary. In some embodiments, the metastatic cancer comprises metastasis of leukemia to the lymph nodes, the lung, the liver, the hind limb, the brain, the kidney, and/or the spleen. In some embodiments, the metastatic cancer comprises metastasis of liver cancer to the intestine, the spleen, the pancreas, the stomach, the lung, and/or the kidney. In some embodiments, the metastatic cancer comprises metastasis of lymphoma to the kidney, the ovary, the liver, the bladder, and/or the spleen.

In some embodiments, the metastatic cancer comprises metastasis of hematopoietic cancer to the intestine, the lung, the liver, the spleen, the kidney, and/or the stomach. In some embodiments, the metastatic cancer comprises metastasis of melanoma to lymph nodes and/or the lung. In some embodiments, the metastatic cancer comprises metastasis of pancreatic cancer to the mesentery, the ovary, the kidney, the spleen, the lymph nodes, the stomach, and/or the liver. In some embodiments, the metastatic cancer comprises metastasis of prostate cancer to the lung, the pancreas, the kidney, the spleen, the intestine, the liver, the bone, and/or the lymph nodes. In some embodiments, the metastatic cancer comprises metastasis of ovarian cancer to the diaphragm, the liver, the intestine, the stomach, the lung, the pancreas, the spleen, the kidney, the lymph nodes, and/or the uterus. In some embodiments, the metastatic cancer comprises metastasis of myeloma to the bone.

In some embodiments, the metastatic cancer comprises metastasis of lung cancer to the bone, the brain, the lymph nodes, the liver, the ovary, and/or the intestine. In some embodiments, the metastatic cancer comprises metastasis of kidney cancer to the liver, the lung, the pancreas, the stomach, the brain, and/or the spleen. In some embodiments, the metastatic cancer comprises metastasis of bladder cancer to the bone, the liver and/or the lung. In some embodiments, the metastatic cancer comprises metastasis of thyroid cancer to the bone, the liver and/or the lung.

In some embodiments, the methods disclosed herein comprise treating cancer-induced bone pain (CIBP) in a subject having metastasis of a cancer to the bone. In some embodiments, the subject has metastasis of prostate cancer, breast cancer, lung cancer, or myeloma to the bone. In some embodiments, the subject is identified as having metastasis to the bone by the use of any one of the following methods: plain film radiography, computed tomography, technetium 99m bone scan, magnetic resonance imaging, fluorodeoxyglucose positron emission tomography, fluorine positron emission tomography, and/or choline positron emission tomography, but is not yet feeling cancer-induced bone pain. In some embodiments, the subject is suffering from cancer-induced bone pain, which is indicative of metastasis of a previously treated or untreated primary tumor to the bone. In some embodiments, the cancer has metastasized to vertebrae, pelvis, long bones, or ribs.

In some embodiments, administration of the composition diminishes the severity of, delays the onset of, or eliminates a symptom of cancer. In some embodiments, the symptom of cancer is cancer-induced bone pain (CIBP). In some embodiments, the CIBP is neuropathic pain. In some embodiments, the CIBP is inflammatory pain. In some embodiments, the CIBP is spontaneous pain. In some embodiments, the symptom of cancer is nociceptive hypersensitivity. In some embodiments, the symptom of cancer is allodynia. In some embodiments, the allodynia is tactile allodynia. In some embodiments, the tactile allodynia is static mechanical allodynia. In some embodiments, the tactile allodynia is dynamic mechanical allodynia. In some embodiments, the subject has bone cancer or metastasis to the bone.

In yet another embodiment of the present invention, a method for treating lupus nephritis (LN) in a subject in need thereof is provided. The method comprises administering to the subject for an administration period, a pharmaceutical composition comprising an effective amount of a compound of Formula (I), (Ia), (Ib), or a pharmaceutically acceptable salt thereof.

Rheumatoid arthritis (RA) is characterized by inflammation and thickening of the joint capsule, together with an effect on the underlying bone and cartilage. Currently, the cause of RA is unknown and no satisfactory cure for RA is available. While a number of therapeutic agents have been developed and utilized to alleviate pain and inflammation associated with the disease, such as disease-modifying antirheumatic drugs (DMARDs) and non-steroidal anti-inflammatory agents (NSAIDs), they often produce intolerable side effects. To addresses this and other needs, the present invention, in one embodiment, provides a method for treating RA using reversible inhibitors of DPP1 of Formula (I), (Ia), (Ib) or pharmaceutically acceptable salts thereof. In one embodiment, a method of for treating RA in a subject in need thereof is provided, and comprises administering to the subject for an administration period, a pharmaceutical composition comprising an effective amount of a compound of Formula (I), (Ia), (Ib), or a pharmaceutically acceptable salt thereof. In a further embodiment, the method comprises reducing neutrophilic inflammation in the subject.

Inflammatory bowel disease (IBD) is a group of inflammatory conditions that affect the colon and small intestine. The most common IBDs are Crohn's disease and ulcerative colitis. The present invention, in one embodiment, addresses the need for novel IBD therapies. Specifically, in one embodiment, a method for treating an inflammatory bowel disease (IBD) in a subject in need thereof is provided. The method comprises administering to the subject for an administration period, a pharmaceutical composition comprising an effective amount of a compound of Formula (I), (Ia), (Ib), or a pharmaceutically acceptable salt thereof.

In a further embodiment, the IBD is Crohn's disease or ulcerative colitis. In even a further embodiment, the method comprises reducing neutrophilic inflammation in the subject.

The length of the administration period in any given case may depend on the nature and severity of the condition being treated and/or prevented and be determined by the physician. In one embodiment, the administration period starts at about the time of condition/disease diagnosis and continues for the lifetime of the patient.

In some embodiments, the administration period is about 30 days, about 35 days, about 40 days, about 45 days, about 50 days, about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months, about 30 months, about 36 months, about 4 years, about 5 years, about 10 years, about 15 years or about 20 years. In some embodiments, the compounds or compositions disclosed herein may be administered for a period of about 24 weeks. In some embodiments, the compounds or compositions disclosed herein may be administered for a period of about 52 weeks. In yet another embodiment, the administration period is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, at least about 24 months, at least about 30 months, at least about 36 months, at least about 4 years, at least about 5 years, at least about 10 years, at least about 15 years or at least about 20 years.

In some embodiments, the administration period for the methods provided herein is at least about 30 days, at least about 35 days, at least about 40 days, at least about 45 days, at least about 50 days, at least about 2 months, at least about 3 months, at least about 4 months or at least about 6 months, at least about 7 months, at least about 8 months, at least about 9 months, at least about 10 months, at least about 11 months, at least about 1 year, at least about 2 years, at least about 3 years, at least about 4 years, at least about 5 years. The administration period for the methods provided herein, in another embodiment, is from about 30 days to about 180 days. In another embodiment, the administration period is from about 30 days to about 36 months, or from about 30 days to about 30 months, or from about 30 days to about 24 months, or from about 30 days to about 18 months, or from about 30 days to about 12 months, or from about 30 days to about 6 months, or from about 6 months to about 30 months, or from about 6 months to about 24 months, or from about 6 months to about 18 months, or from about 12 months to about 36 months, or from about 12 months to about 24 months.

In one embodiment, the administration period is from about 1 year to about 30 years. For example, the administration period, in one embodiment, is from about 1 year to about 25 years, 1 year to about 20 years, from about 1 year to about 15 years, from about 1 year to about 10 years, from about 1 year to about 5 years, from about 1 year to about 3 years, from about 1 year to about 2 years, from about 2 years to about 15 years, from about 2 year to about 10 years, from about 2 years to about 8 years, from about 2 year to about 5 years, from about 2 years to about 4 years, or from about 2 years to about 3 years.

In one embodiment of the method, the subject is administered the composition once daily during the administration period. In another embodiment, the patient is administered the composition twice daily, or every other day, or once a week during the administration period. In another embodiment, administration is every other day, every third day, 3× per week or 4× per week during the administration period.

In a preferred embodiment, the oral dosage form is administered once daily during the administration period. In a further embodiment, the oral dosage form is administered at approximately the same time every day, e.g., prior to breakfast. In another embodiment, the composition comprising an effective amount of Formula (I), (Ia), (Ib) is administered 2× daily during the administration period. In yet another embodiment, the composition comprising an effective amount of Formula (I), (Ia), (Ib) is administered 1× per week, every other day, every third day, 2× per week, 3× per week, 4× per week, or 5× per week during the administration period.

Administration, in one embodiment, is via the oral route. In a further embodiment, the composition is administered once daily.

### Examples

The present disclosure is further illustrated by reference to the following Examples. However, it should be noted that the Examples, like the embodiments described above, are illustrative and are not to be construed as restricting the scope of the invention in any way.

### Glossary of Abbreviations

Burgess reagent: (methoxycarbonylsulfamoyl) triethylammonium hydroxide
Pd(dppf)Cl₂: [1,1′-bis(diphenylphosphino) ferrocene]dichloropalladium (II) X-Phos: 2-dicyclohexylphosphino-2′,4′,6′-triisopropylbiphenyl
DMF: N,N-dimethylformamide
HATU: 2-7-aza-1 h-benzotriazole-1-yl-1,1,3,3-tetramethyluronium hexafluorophosphate
DIPEA: N,N-diisopropylethylamine
LDA: lithium diisopropylamide
PE: petroleum ether
EA: ethyl acetate
THF: tetrahydrofuran
MeOH: methanol
DCM: dichloromethane
TMSOTf: trimethylsilyl trifluoromethanesulfonate

### Example 1-Prophetic Syntheses of Compounds of Formula (I) and Key Intermediates

In one embodiment, INT-1: tert-butyl(S)-(1-cyano-2-(4-(4,4,5,5-tetramethyl-1,3,2-dioxaborolan-2-yl)phenyl)ethyl) carbamate, is used as a reactant to obtain one of the compounds described herein.

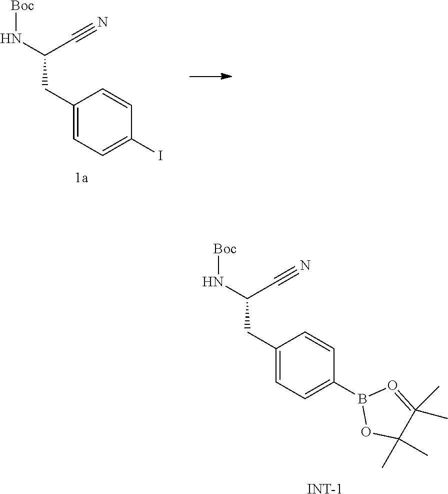

Compound 1a (for preparation method refer to WO 2013/041497) is dissolved in 1,4-dioxane, and pinacolborane, triethylamine and Pd (dppf) Cl₂ added then reacted at 100° C. microwave conditions for 1 hour, then filtered after the reaction ended, the filtrate concentrated, and the residue subjected to separation and purification by silica gel column chromatography (eluent ratio PE: EA (v/v)=1:0-10:1), yielding INT-1, a white solid.

In one embodiment, INT-2: tert-butyl(S)-(1-cyano-2-(2-fluoro-4-(4,4,5,5-tetramethyl-1,3,2-dioxaborolan-2-yl)phenyl)ethyl) carbamate, is used as a reactant to obtain one of the compounds described herein.

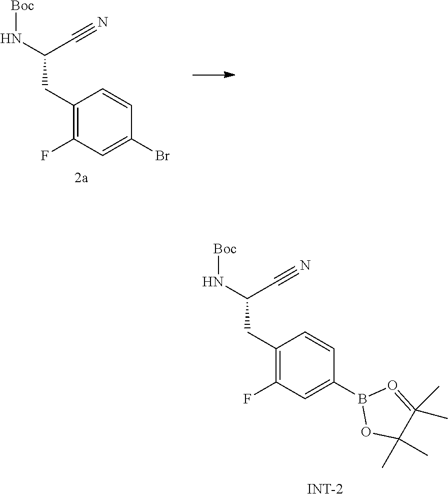

Compound 2a (2.0 g, 5.8 mmol, for preparation method refer to WO 2016/016242) is dissolved in glycol dimethyl ether, then after bis(pinacolato)diboron, potassium acetate, Pd (dppf) Cl₂ are added the temperature is raised to 90° C. and the reaction is continued for 3 hours, after the reaction ends filtration is carried out, the filtrate is concentrated, and the residue obtained is subjected to separation and purification by silica gel column chromatography.

In one embodiment, (S)—N—((S)-1-cyano-2-(2-fluoro-4-(3-methyl-2-oxo-2,3-dihydrobenzo[d] oxazol-5-yl)phenyl)ethyl)-1,4-oxazepane-2-carboxamide (compound 1) is synthesized according to the following scheme.

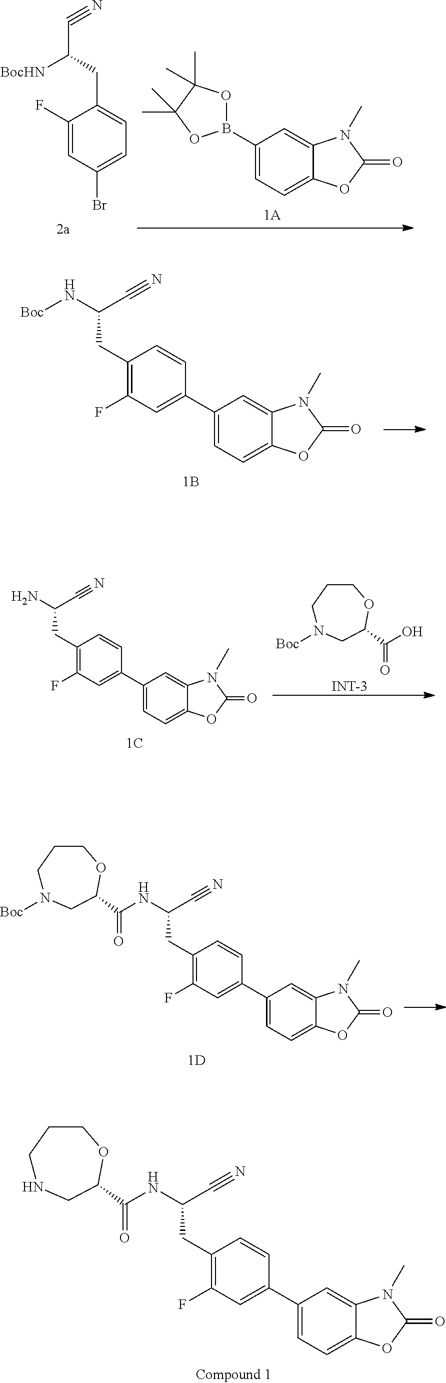

Compound 1A (for synthesis method refer to WO 2016/016242, incorporated by reference herein in its entirety) is dissolved in 1,4-dioxane and water. Then, intermediate 2a, potassium carbonate, and Pd (dppf) Cl₂ DCM are added and are reacted at 90° C. for 3 hours. The mixture is cooled to room temperature and a saturated aqueous solution of sodium chloride is then added. Ethyl acetate is used for extraction, and the organic phase is combined, and dried with anhydrous sodium sulphate, and is filtered and concentrated and the residue is subjected to separation and purification by silica gel column chromatography, yielding the compound 1B as a white solid.

Compound 1B is dissolved in formic acid, and is allowed to react overnight at room temperature. After concentrating until dry, ethyl acetate is added, a saturated aqueous solution of sodium bicarbonate is dripped in to adjust pH to about 8, and the organic layer is separated. Extraction is then carried out using ethyl acetate, the organic phase is combined and is dried with anhydrous sodium sulphate, is filtered, and is concentrated, yielding compound 1C as a pale yellow solid.

Compound 1C is dissolved in DMF. After, DIPEA, HATU and INT-3 (preparation method according to WO 2015/110826, the contents of which are incorporated by reference in its entirety) are added, they are reacted overnight at room temperature. A saturated aqueous solution of ammonium chloride is dripped in and an extraction reaction takes place, a saturated aqueous solution of sodium chloride is added, and ethyl acetate is used for extraction, the organic phase is washed using a saturated aqueous solution of sodium chloride, and is dried with anhydrous sodium sulphate, is filtered and concentrated, and yields the compound 1D as a light yellow solid, which is used directly in the next reaction step.

Compound 1D is dissolved in formic acid, and is reacted for 10 minutes at 50° C. The product is concentrated until dry, and ethyl acetate is added. A saturated aqueous solution of sodium bicarbonate is then added to adjust the pH to about 8, and the organic layer is separated. Ethyl acetate is used for extraction, and the organic phase is combined, anhydrous sodium sulphate is used for drying, and filtration and concentration is carried out. The residue is subjected to separation and purification, e.g., by silica gel column chromatography.

In one embodiment, N—((S)-1-cyano-2-(2-methoxy-4-(3-methyl-2-oxo-2,3-dihydrobenzo[d] oxazol-5-yl)phenyl)ethyl)-1,4-oxazepane-2-carboxamide (compound 2), is synthesized as follows:

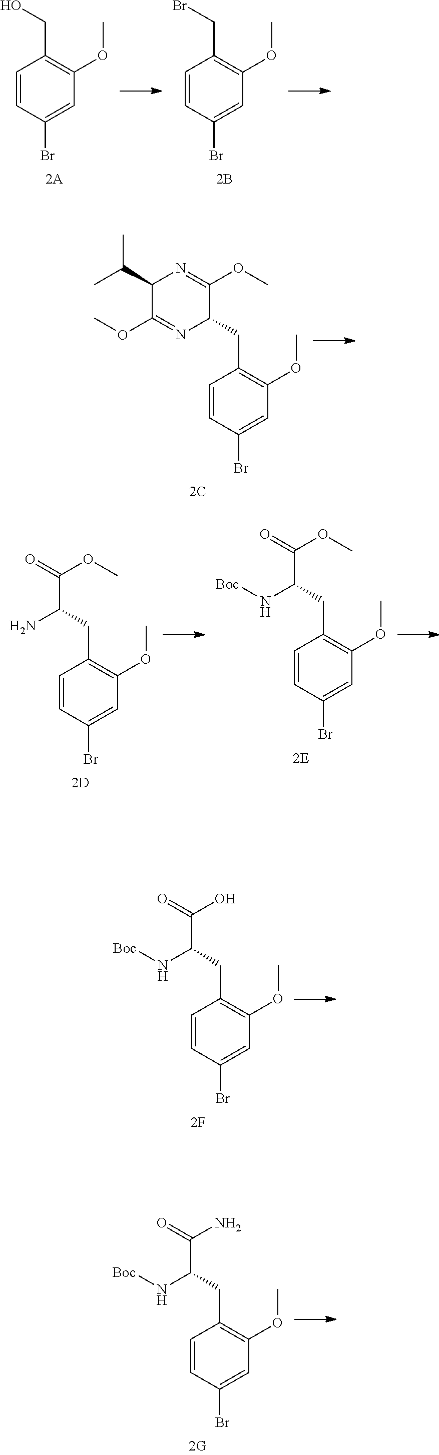

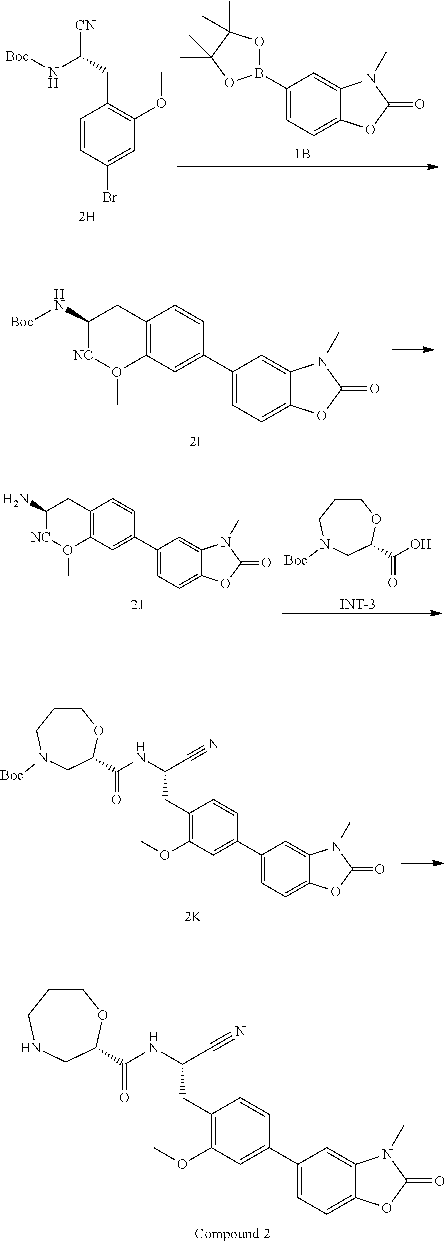

Compound 2A is dissolved in DCM, and CBr₄ is then added. Then, PPh₃ is slowly added, and reacted at room temperature for 1 hour. PE/EA (v/v=5:1) is added to the reaction liquid, stirred, suction filtered, and the filtrate is concentrated, and the residue is subjected to separation and purification by silica gel column chromatography (PE:EA (v/v)=30:1-20:1) yielding the compound 2B as a white solid.

(R)-2,5-dihydro-3,6-dimethoxy-2-isopropylpyrazine is dissolved in THF (20 mL), the atmosphere is replaced with nitrogen, and the temperature is reduced to −78° C. n-butyllithium is added, and they are reacted for 1 hour. Then, a THF solution of 2B (1.67 g, 5.97 mmol) added, then the reaction continues for 2 hours at −78° C. Then, a saturated NH₄Cl solution is used to quench the reaction, followed by ethyl acetate (EA) extraction. The organic phase is combined, and the organic phase is washed using domestic salt water, and then is dried with anhydrous sodium sulphate, and is concentrated, then after passing through the silica gel column (EA/PA (v/v)=0%-6%), the compound 2C, with a colourless oily appearance, is obtained.

Compound 2C is dissolved in acetonitrile, then 1M HCl is added, and these are allowed to react overnight at room temperature. The system is centrifuged dry, and a saturated aqueous solution of sodium bicarbonate is added to neutralize the system to weakly alkaline, and EA is added to carry out liquid separation extraction. The organic phase is combined, and the organic phase is washed with saturated domestic salt water, and is dried with anhydrous sodium sulphate, and is concentrated and the residue is subjected to separation and purification by silica gel column chromatography, yielding compound 2D.

Compound 2D is dissolved in DCM, and triethylamine and di-tert-butyl dicarbonate ester is added and are reacted at room temperature for 3 hours, then water is added to the system, and liquid separation extraction is carried out, and the organic phase is combined, and the organic phase is washed with saturated domestic salt water, and is dried with anhydrous sodium sulphate, is concentrated, and the residue is subjected to separation and purification by silica gel column chromatography, yielding compound 2E, with a colourless oily appearance.

Compound 2E (0.7 g, 1.87 mmol) is dissolved in methanol and water, NaOH is added, and reacted for 2 hours at room temperature, then the methanol is centrifuged off, and dilute hydrochloric acid is used to adjust the pH to weak acidity, EA is added to carry out liquid separation extraction, and the organic phase is centrifuged dry, and this yields compound 2F.

Compound 2F, NH₄Cl and HATU are dissolved in DMF, and DIPEA is added, and are reacted overnight at room temperature, water and EA are added and liquid separation extraction is carried out, the organic phase is dried and concentrated with anhydrous Na₂SO₄, and this yields a crude resultant product, compound 2G.

Compound 2G is dissolved in DCM, Burgess reagent is added, and is reacted overnight at room temperature. The system is centrifuged to concentrate, and the raw product is subjected to silica gel column chromatography separation and purification, and this yields compound 2H.

Compound 2H, compound 1B, Pd (dppf) Cl₂· DCM, and potassium carbonate are dissolved in 1,4-dioxane. The system undergoes nitrogen atmosphere replacement protection, and is reacted at 90° C. for 3 hours. Concentration centrifuging is carried out, then dissolving in DCM, diatomite suction filtration is carried out. The filtrate is centrifuged dry, and the raw product is subjected to silica gel column chromatography separation. This yields compound 2I as a pale yellow solid.

Compound 2I is dissolved in formic acid, and the mixture is stirred at room temperature overnight. DCM is added to the system to dilute, and saturated sodium bicarbonate solution is used to adjust to mildly acidic, then liquid separation is used to separate DCM from water, and the organic phase is dried and concentrated and centrifuged dry, and this yields a crude resultant product, compound 2J.

Compound 2J and INT-3 are dissolved in DMF, HATU DIPEA are added, and are reacted overnight at room temperature. Water and EA are added to the reaction system to carry out liquid separation extraction, and the organic phase is dried and concentrated, and this yields a pale yellow oily crude resultant product, which is then subjected to MeOH/DCM (v/v)=0-₁₀%, and this yields compound 2K.

2K (90 mg, 0.16 mmol) is dissolved in formic acid (5 ml), and is reacted at room temperature for 4 hours. DCM is added to dilute the system, and a saturated sodium bicarbonate solution is used to adjust to weak alkalinity, then DCM and water liquid separation is carried out, and the organic phase is dried and concentrated and centrifuged dry. This yields a residue, and then the residue is subjected to separation and purification by silica gel column chromatography, yielding the compound 2.

In one embodiment, (S)—N—((S)-1-cyano-2-(3-fluoro-4-(3-methyl-2-oxo-2,3-dihydrobenzo[d] oxazol-5-yl)phenyl)ethyl)-1,4-oxazepane-2-carboxamide (compound 3), is synthesized according to the following scheme.

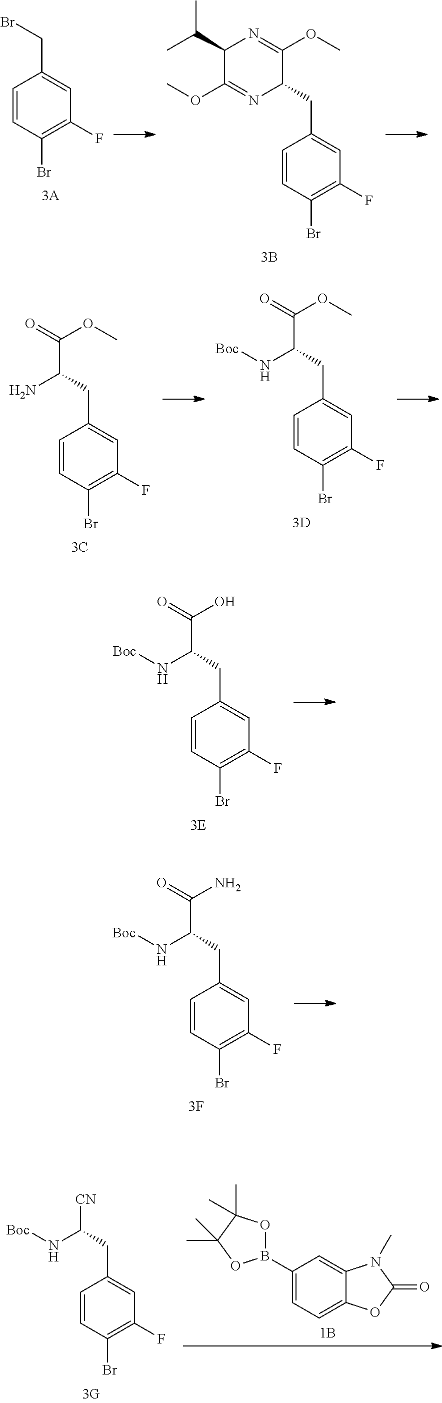

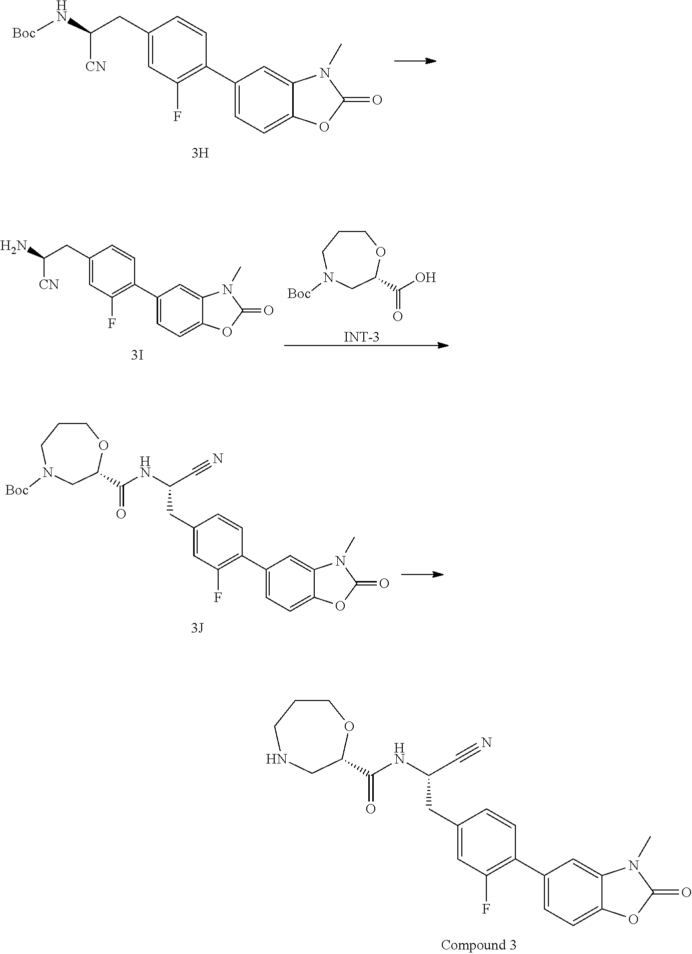

Compound 3 is prepared from compound 3A with reference to the preparation method of compound 2, above.

In one embodiment, (S)—N—((S)-1-cyano-2-(5-(1-methyl-2-oxoindolin-6-yl)thiophen-2-yl)ethyl)-1,4-oxazepane-2-carboxamide (compound 4) is synthesized according to the following scheme:

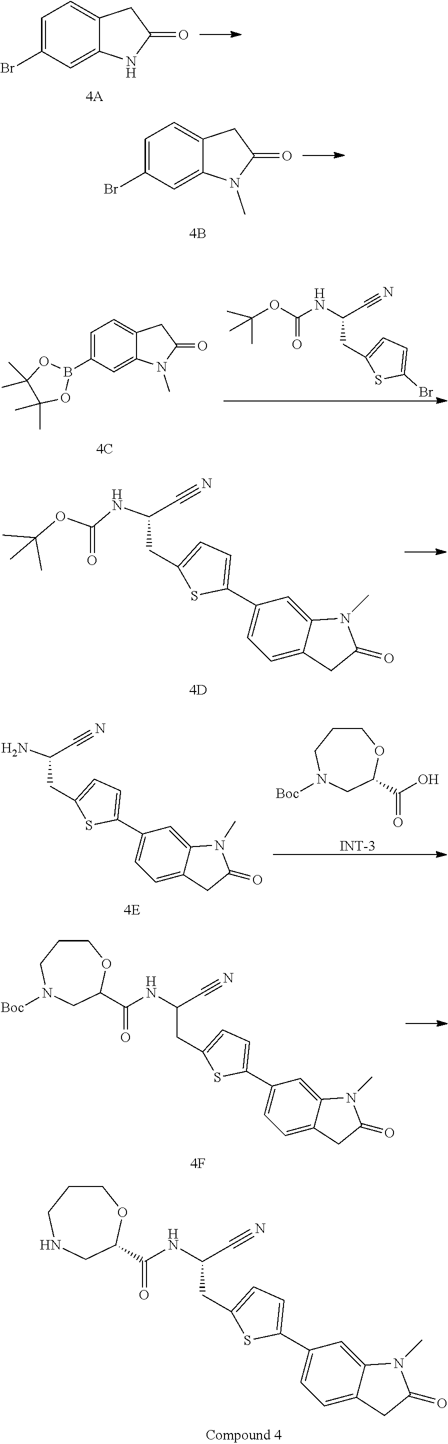

Compound 4A is dissolved in acetonitrile, and potassium carbonate (23 g, 94.32 mmol), methyl iodide (2.96 ml, 47.16 mmol) are added and heated to 70° C. and stirred overnight. The reaction liquid is concentrated, then extraction is carried out using DCM and water, the organic phase is dried and concentrated, and is subjected to column chromatography separation, and this yields compound 4B as a brown solid.

Compound 4B, palladium acetate, potassium acetate, XPHOS, bis(pinacolato)diboron are mixed and dissolved in 1,4-dioxane, and are heated to 95° C. in a protective nitrogen atmosphere, and are reacted for 3 hours. Once LCMS detects the reaction is complete, the reaction liquid is concentrated, and column chromatography is used for separation. This yields compound 4C as a pale yellow solid.

N-[(1S)-2-(5-thiophene-2-yl)-1-cyanoethyl]carbamic acid tert-butyl ester, compound 4C, potassium carbonate and Pd (dppf) Cl₂· DCM is mixed and dissolved in 1,4-dioxane and water, and are microwaved to heat to 120° C., and are reacted for 1 hour. After the reaction has finished (e.g., detected by LCMS), the reaction liquid is concentrated, and column chromatography separation is carried out. This yields compound 4D as a pale yellow solid.

Compound 4D is added to formic acid, and stirred for 3 hours at room temperature. After LCMS detects the reaction is complete, a saturated sodium carbonate solution is added to the reaction liquid to adjust pH to 10, then extraction with DCM is carried out. The organic phase is dried and is concentrated. This yields the compound 4E.

Compound 4E (119 mg, 0.4 mmol), INT-3 (98 mg, 0.4 mmol), HATU (180 mg, 0.48 mmol) and DIEA (0.13 ml, 0.8 mmol) are dissolved in DMF, and stirred at room temperature overnight. After LCMS detects the reaction is complete, water and EA are added and extraction is carried out, the organic phase is dried and is concentrated, and is subjected to column chromatography separation. This yields compound 4F as a yellow solid 4F.

Compound 4F is dissolved in formic acid, and is reacted at room temperature for 3 hours. After LCMS detects the reaction is complete, the reaction liquid is poured into a saturated sodium carbonate solution, and the pH is adjusted to 10. EA extraction is carried out, and the organic phase is dried and concentrated, then separation and purification by silica gel column chromatography is carried out, yielding compound 4.

In one embodiment, (S)—N—((S)-1-cyano-2-(5-(3-methyl-2-oxo-2,3-dihydrobenzo[d] oxazol-5-yl)thiophen-2-yl)ethyl)-1,4-oxazepane-2-carboxamide (compound 5) is synthesized according to the following scheme.

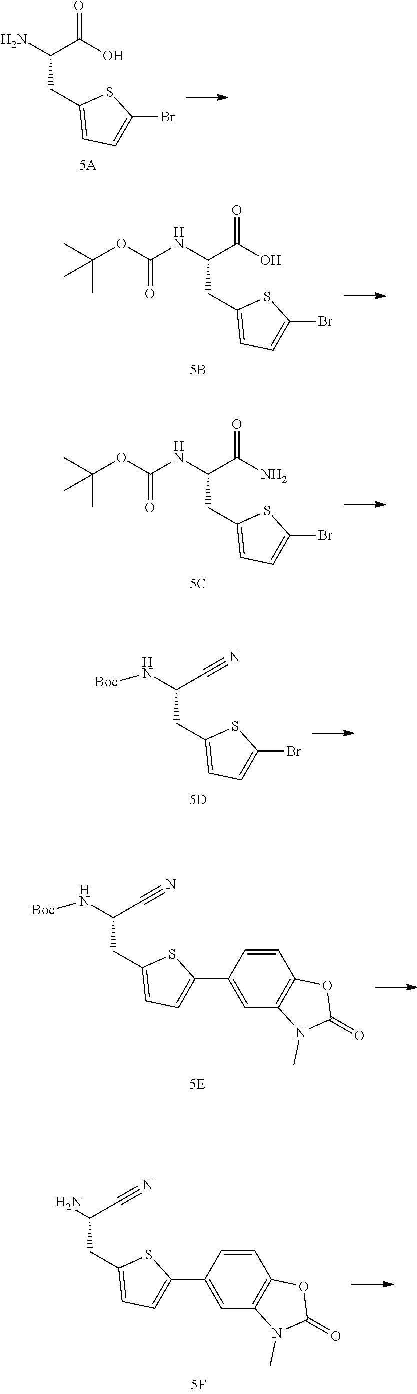 [SMILES: N[C@@H](Cc1ccc(Br)s1)C(=O)O ; CC(C)(C)OC(=O)N[C@@H](Cc1ccc(Br)s1)C(=O)O ; CC(C)(C)OC(=O)N[C@@H](Cc1ccc(Br)s1)C(N)=O ; CC(C)(C)OC(=O)N[C@H](C#N)Cc1ccc(Br)s1 ; Cn1c(=O)oc2ccc(-c3ccc(C[C@@H](C#N)NC(=O)OC(C)(C)C)s3)cc21 ; Cn1c(=O)oc2ccc(-c3ccc(C[C@H](N)C#N)s3)cc21]

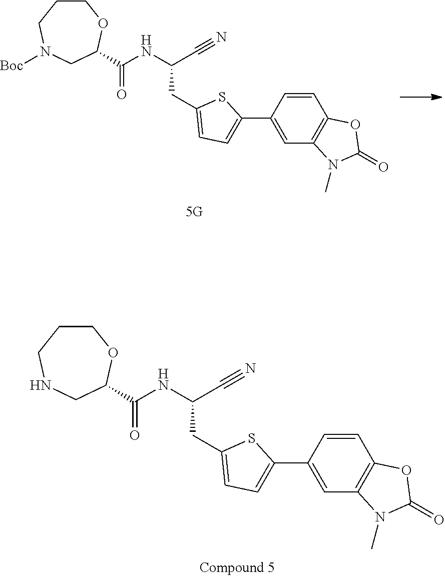 [SMILES: Cn1c(=O)oc2ccc(-c3ccc(C[C@@H](C#N)NC(=O)[C@@H]4CN(C(=O)OC(C)(C)C)CCCO4)s3)cc21 ; Cn1c(=O)oc2ccc(-c3ccc(C[C@@H](C#N)NC(=O)[C@@H]4CNCCCO4)s3)cc21]

In one embodiment, (S)—N—((S)-2-(4-(7-acetamido-2,3-dihydro-1H-inden-4-yl)-2-fluorophenyl)-1-cyanoethyl)-1,4-oxazepane-2-carboxamide (compound 6) is synthesized according to the following synthesis route.

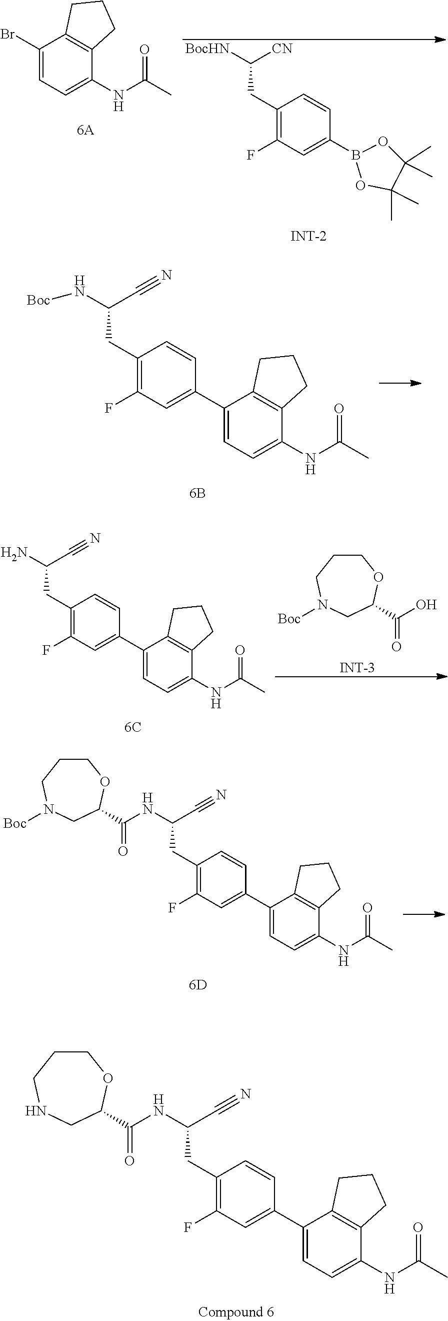

Compound 5A is dissolved in methanol, and triethylamine is added, then di-tert-butyl dicarbonate ester is added and reacted at room temperature for 2 hours, then after the mixture is concentrated, dilute hydrochloric acid (1N) is used to adjust pH=6-7. Dichloromethane is used for extraction, and then the concentration is repeated and separation and purification by silica gel column chromatography is carried out, yielding the compound 5B, as a yellow solid.

Compound 5B is dissolved in DMF, ammonium chloride, 2-(1H-benzotriazole-1-yl)-1,1,3,3-tetramethyluronium tetrafluoroborate and DIPEA are added to the reaction vessel, and the mixture is reacted overnight at room temperature. Water is then added, and ethyl acetate (EA) is used for extraction. Then, washing with water takes place, and anhydrous sodium sulphate is used to dry and concentrate. Separation and purification by silica gel column chromatography is then carried out. This yields compound 5C as a pale yellow solid.

Compound 5C is dissolved in dichloromethane. Burgess reagent is added in an ice bath, and the reaction continues for 2 hours. Water is added, and ethyl acetate is used for extraction, then after drying and concentration with anhydrous sodium sulphate, separation and purification by silica gel column chromatography is carried out, and yields compound 5D as a pale yellow solid.

Compound 5D is dissolved in dioxane, and 3-methyl-5-(4,4,5,5-tetramethyl-1,3,2-dioxaborolan-2-yl)-benzo[d] oxazole-2 (3H), potassium carbonate and Pd (dppf) Cl₂ are added, then water is added. The mixture is heated to 100° C. with a microwave and reacted for 12 hours. Concentration, separation, and purification by silica gel column chromatography are then carried out. This yields compound 5E as a yellow solid.

Compound 5E is dissolved in formic acid, and then reacted at room temperature for 3 hours, then a saturated aqueous solution of sodium carbonate is used to adjust pH=7-8, and dichloromethane is used for extraction. The organic phase is combined, and the organic phase is washed with a saturated aqueous solution of sodium chloride, then dried with anhydrous sodium sulphate. Concentration, separation and purification by silica gel column chromatography is carried out. This yields compound 5F as a yellow solid.

Compound 5F is dissolved in DMF. HATU, DIEA, (S)-4-(t-butyloxycarbonyl)-1-4-oxepane-2-carboxylic acid are added in sequence, and reacted at room temperature for 12 hours. Water is added, and ethyl acetate is used for extraction, the organic phase is combined, and the organic phase is washed with water, then saturated domestic salt water is used to wash, then anhydrous sodium sulphate is used to dry. Concentration, separation and purification by silica gel column chromatography is carried out and this yields compound 5G as a yellow solid.

Compound 5G is dissolved in formic acid, and is reacted at room temperature for 3 hours. Then a saturated aqueous solution of sodium carbonate is used to adjust pH=7-8, and dichloromethane is used for extraction, then the organic phase is combined, and the organic phase is washed with a saturated aqueous solution of sodium chloride, then is dried with anhydrous sodium sulphate. Concentration, separation and purification by silica gel column chromatography is carried out and this yields compound 5.

In one embodiment, (S)—N—((S)-1-cyano-2-(4-(1,1-dioxido-2,3-dihydrobenzo[b] thiophen-5-yl)-2-fluorophenyl)ethyl)-1,4-oxazepane-2-carboxamide (compound 7) is synthesized according to the following synthesis route.

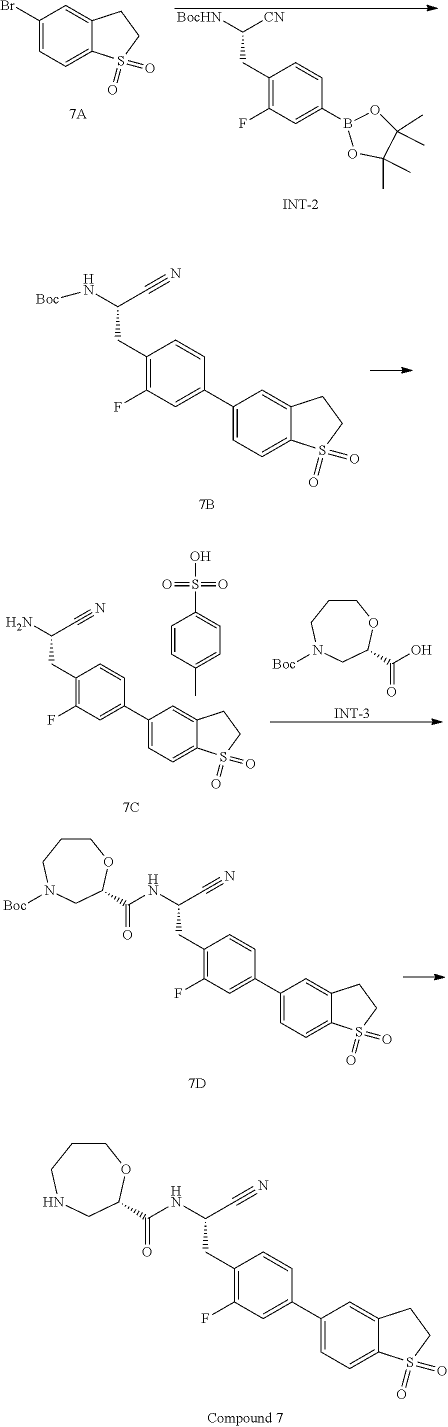

Compound 7A (see, e.g., the preparation method according to EP3342765, incorporated by reference herein in its entirety), INT-2, Pd (dppf) Cl₂· DCM potassium carbonate (0.33 g, 2.43 mmol) are dissolved in 1,4-dioxane. Water is added and then nitrogen is used to replace the atmosphere 3 times, then these are reacted under nitrogen for 4 hours at 90° C. The reaction liquid is concentrated until dry, and dichloromethane is added to dissolve. Filtration and concentration is carried out, then the residue is subjected to separation and purification by silica gel column chromatography. This yields the compound 7B, as a white solid.

Compound 7B is dissolved in acetonitrile, and p-toluenesulfonic acid is added. The mixture is reacted at room temperature for 16 hours, and is filtered. The filter cake is then rinsed once with acetonitrile, and the filter cake is centrifuged dry. This yields compound 7C, as a white solid.

INT-3 is dissolved in dichloromethane, then triethylamine and HATU are added. The mixture is reacted while stirring at room temperature for 1 hour. Then, compound 7C is added, and left at room temperature overnight. After the reaction finishes, concentration is carried out, and the raw product 7D is obtained.

The raw product 7D is dissolved in acetonitrile, and p-toluenesulfonic acid is added, then reacted for 16 hours at room temperature, is concentrated until dry, and ethyl acetate is added. A saturated aqueous solution of sodium bicarbonate is dripped in to adjust pH to around 8, and the organic layer is separated. Ethyl acetate is used for extraction, and after combination, the organic phase is dried using anhydrous sodium sulphate, is filtered and concentrated. The residue is subjected to separation and purification by silica gel column chromatography. This yields compound 7.

In one embodiment, (S)—N—((S)-1-cyano-2-(3-fluoro-4′-(pentafluoro-16-sulfaneyl)-[1,1′-biphenyl]-4-yl)ethyl)-1,4-oxazepane-2-carboxamide (compound 8) is synthesized according to the following synthesis route.

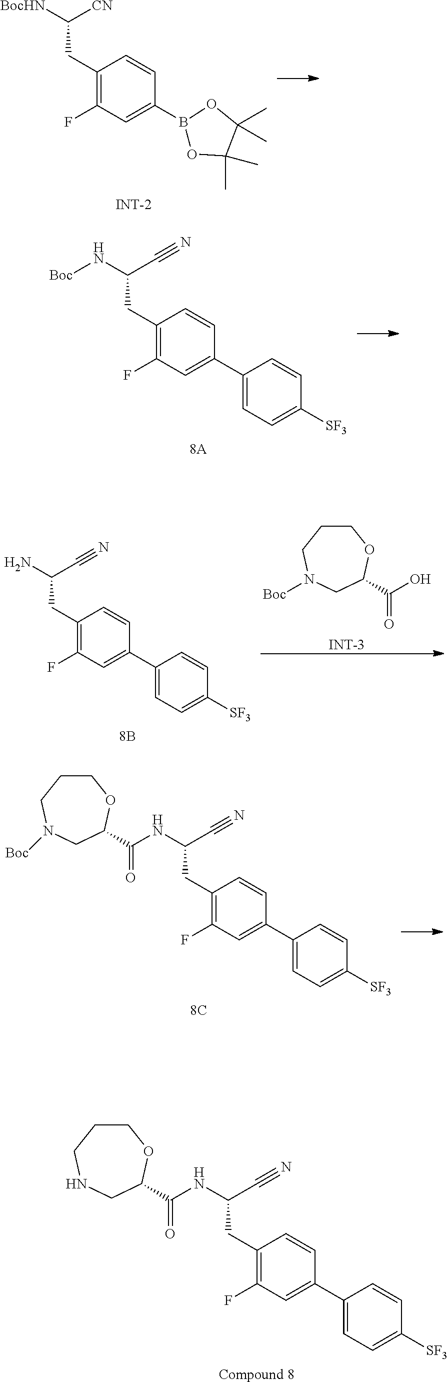

INT-2 is dissolved in dioxane, and 4-bromophenylsulfur pentafluoride, potassium carbonate and Pd (dppf) Cl₂ are added. Then, water is added, and the reaction is carried out under protective nitrogen at 100° C. for 4 hours. Cconcentration, separation and purification is carried out by silica gel column chromatography. This yields compound 8A.

Compound 8A is dissolved in acetonitrile. Then, p-toluenesulfonic acid is added, and reacted at 30° C. for 2 hours, water (30 ml) is added, and a saturated aqueous solution of sodium carbonate is used to adjust to pH=7-8. Dichloromethane is used for extraction. Then, the product is washed with saturated domestic salt water, is dried, and is concentrated with anhydrous sodium sulphate. This yields compound 8B.

Compound 8B is dissolved in DMF. Then, HATU, DIPEA and INT-3 are added sequentially, and are reacted at room temperature for 12 hours. Next, water is added, ethyl acetate is used for extraction. The organic phase is combined, and then the organic phase is washed in water. Then, saturated domestic salt water is used for washing, anhydrous sodium sulphate is used for drying. Concentration, separation and purification by silica gel column chromatography is carried out, and this yields compound 8C.

Compound 8C is dissolved in acetonitrile, p-toluenesulfonic acid (220 mg, 1.26 mmol) is added, and the mixture is reacted for 3 hours at 30° C. A saturated aqueous solution of sodium carbonate is used to adjust pH=7-8, and dichloromethane is used for extraction. Then the organic phase is combined, and the organic phase is washed using a saturated aqueous solution of sodium chloride. Then, the product is dried with anhydrous sodium sulphate. Concentration, separation and purification by silica gel column chromatography is carried out, and this yields compound 8.

In one embodiment, (S)—N—((S)-1-cyano-2-(3-fluoro-3′-(pentafluoro-16-sulfaneyl)-[1,1′-biphenyl]-4-yl)ethyl)-1,4-oxazepane-2-carboxamide (compound 9) is synthesized according to the following synthesis route.

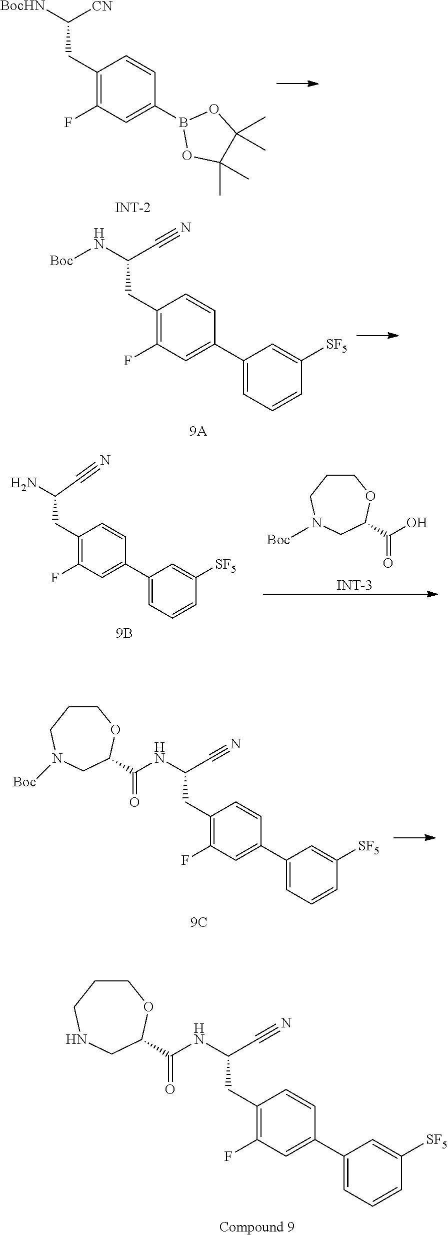

INT-2 is dissolved in dioxane, and 3-bromophenylsulfur pentafluoride, potassium carbonate and Pd (dppf) Cl₂ are added. Then, water is added, then the mixture is reacted at 100° C. for 4 hours in protective nitrogen. Concentration, separation and purification by silica gel column chromatography is carried out. This yields compound 9A.

Compound 9A is dissolved in acetonitrile, and p-toluenesulfonic acid is added, and reacted for 2 hours at 30° C. Then, water is added, and a saturated aqueous solution of sodium carbonate is used to adjust pH=7-8. Dichloromethane is used for extraction, saturated domestic salt water is used for washing. Next, drying with anhydrous sodium sulphate is carried out, followed by a concentration step. Subsequently, compound 9B is obtained.

Compound 9B is dissolved in DMF, then HATU, DIEA and INT-3 are added sequentially, and reacted at room temperature for 12 hours. Water is then added, and ethyl acetate is used for extraction. The organic phase is combined, then the organic phase is washed with water, then is washed again in saturated domestic salt water. Drying with sodium sulphate is carried out, and after concentration, separation and purification by silica gel column chromatography is carried out. This yields compound 9C.

Compound 9C is dissolved in acetonitrile, and p-toluenesulfonic acid is added, and the mixture is reacted at 30° C. for 3 hours. A saturated aqueous solution of sodium carbonate is used to adjust pH=7-8, and dichloromethane is used for extraction, and then the organic phase is combined, and the organic phase is washed with aqueous solution of sodium chloride, is dried with anhydrous sodium sulphate. Concentration, separation and purification by silica gel column chromatography is carried out. This yields compound 9.

In one embodiment, (S)—N—((S)-1-cyano-2-(2-fluoro-4-(1-oxo-1,2,3,4-tetrahydroisoquinolin-6-yl)phenyl)ethyl)-1,4-oxazepane-2-carboxamide (compound 10) is synthesized according to the following synthesis route.

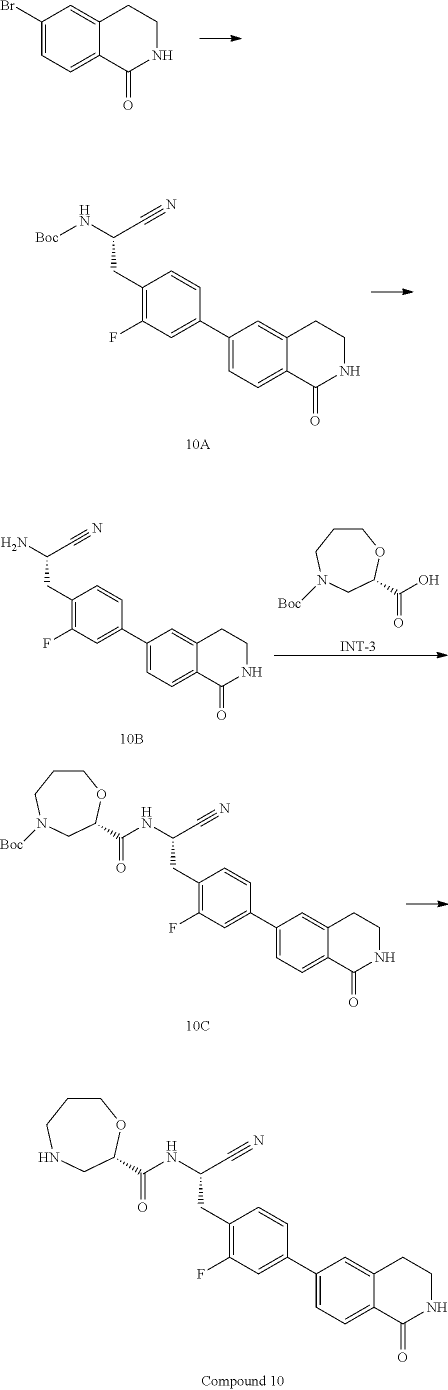

Compound 4A, INT-2, Pd (dppf) Cl₂ and potassium carbonate are added sequentially to 1,4-dioxane and water. The system atmosphere is replaced 3 times with nitrogen, then the mixture is reacted at 100° C. for 2 hours. After the reaction ends, it is cooled to room temperature, and water is added. The aqueous phase is extracted using ethyl acetate, the organic phase is combined and a saturated aqueous solution of sodium chloride is used for washing. The mixture is then dried with anhydrous sodium sulphate, and is filtered. The filtrate is subjected to vacuum concentration. The residue is separated using silica gel column chromatography, and this yields compound 10A as a brown solid.

Compound 10A is dissolved in formic acid, and then reacted at 50° C. for 10 minutes, concentrated until dry. Ethyl acetate is added to the mixture and a saturated aqueous solution of sodium bicarbonate is dripped in to adjust pH to about 8. The organic layer is separated, and the organic phase extracted with ethyl acetate. After the organic phase is combined, anhydrous sodium sulphate is used for drying. Filtration and concentration is carried out and yields compound 10B.

Compound 10B is dissolved in DMF, and INT-3, diisopropylethylamine and HATU are added. The mixture is reacted for 1 hour at room temperature. A saturated aqueous solution of sodium chloride is added, and ethyl acetate is used for extraction. The organic phase is washed using a saturated aqueous solution of sodium chloride, and then dried with anhydrous sodium sulphate, and then is filtered, and is concentrated. The residue is separated with silica gel column chromatography. The process yields compound 10C, as a pale yellow solid.

Compound 10C is dissolved in formic acid, and is reacted for 10 minutes at 50° C. Vacuum concentration is carried out, and ethyl acetate is added, then a saturated aqueous solution of sodium bicarbonate is used to adjust pH to about 8. The organic layer is separated, and ethyl acetate is used for extraction. Then, the organic phase is combined and anhydrous sodium sulphate is used for drying. Filtering and concentration is carried out, and then the residue is subjected to separation and purification by silica gel column chromatography, yielding compound 10.

In one embodiment, (S)—N—((S)-1-cyano-2-(4-(cyclopentylethynyl)-2-fluorophenyl)ethyl)-1,4-oxazepane-2-carboxamide (compound 11) is synthesized according to the following synthesis route.

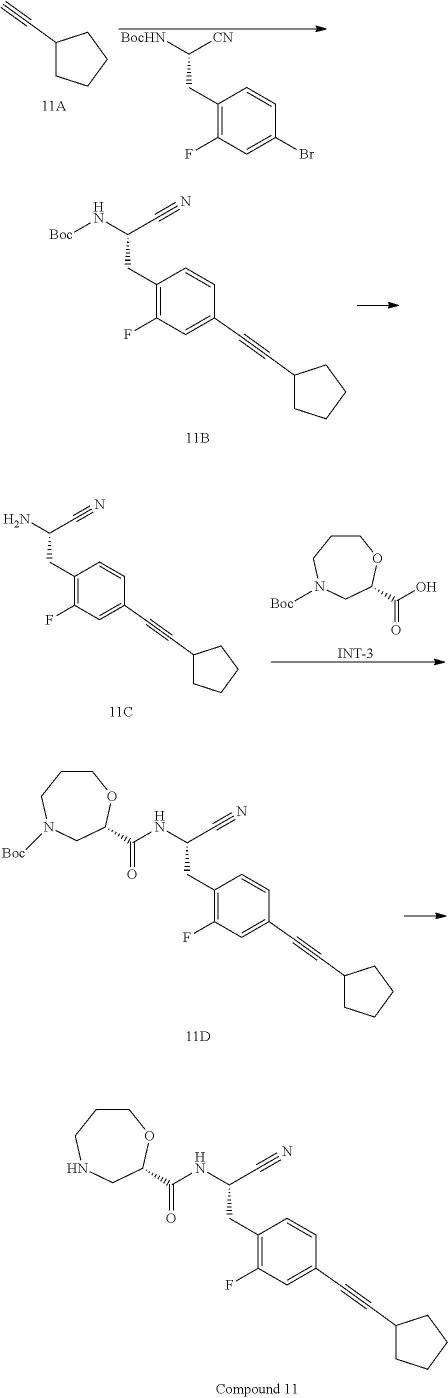

Compound 11A, INT-2, Pd (dppf) Cl₂ and potassium carbonate are placed in a single neck flask, then 1,4-dioxane is added and water is added. Nitrogen is used 3 times for atmosphere replacement, then the reaction takes place at 95° C. for 4 hours. The reaction is cooled to room temperature and is concentrated. Next, separation and purification by silica gel column chromatography is carried out yielding title compound 11B, a white solid.

Compound 11B is dissolved in formic acid, and reacted for 4 hours at 35° C. A saturated potassium carbonate solution is used to adjust the reaction system to alkaline, and ethyl acetate is used for extraction, then drying with anhydrous sodium sulphate, filtration and concentration carried out. This yields compound 11C, a pale yellow oily substance.

Compound 11C is dissolved in DMF, and INT-3, HATU and DIPEA are added, then is reacted overnight at room temperature after addition is complete. Water is added to the system, then ethyl acetate is used for extraction, drying with sodium sulphate takes place, and after filtration and concentration, separation and purification by silica gel column chromatography is carried out yielding compound 11D, a white solid.

Compound 11D is dissolved in formic acid, and reacted at 35° C. for 4 hours. Saturated potassium carbonate is used to adjust the reaction system to alkaline, ethyl acetate is used for extraction, drying with anhydrous sodium sulphate takes place. Filtration and concentration is then carried out. Separation and purification by silica gel column chromatography is carried out and this yields compound 11.

In one embodiment, (S)—N—((S)-1-cyano-2-(4-(5-cyano-4-methylthiazol-2-yl)-2-fluorophenyl)ethyl)-1,4-oxazepane-2-carboxamide (compound 12) is synthesized according to the following synthesis route.

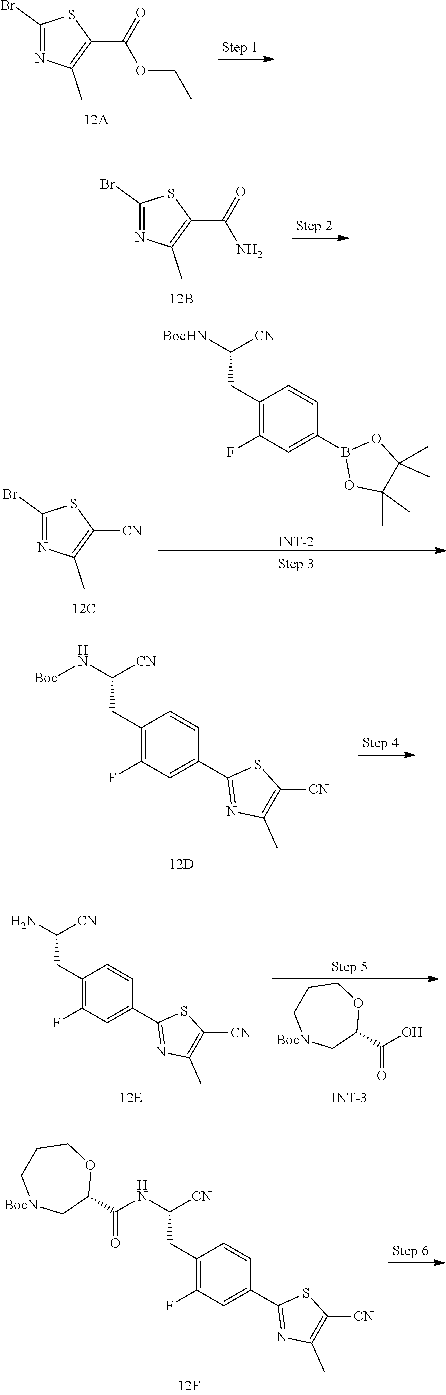

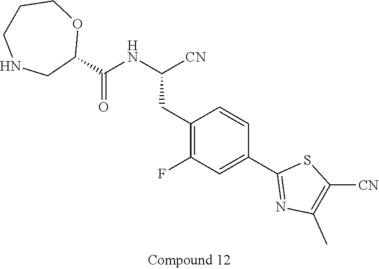 [SMILES: Cc1nc(-c2ccc(C[C@@H](C#N)NC(=O)[C@@H]3CNCCCO3)c(F)c2)sc1C#N]

Compound 12A is dissolved in 30% ammonium hydroxide, and reacted at 35° C. for 24 hours. After cooling to room temperature, ethyl acetate is then used for extraction, and after the organic phase is combined, anhydrous sodium sulphate is used for drying, then after filtration and concentration, compound 12B is obtained, a yellow solid.

Compound 12B is dissolved in dichloromethane, and Burgess reagent is added, then these reacted overnight at room temperature. After concentration, separation and purification by silica gel column chromatography is carried out yielding the title compound 12C, a white solid.

Compound 12C, INT-2, Pd (dppf) Cl₂ and potassium carbonate are added to a single neck flask, then 1,4-dioxane and water are added. After nitrogen atmosphere replacement is carried out 3 times, these are reacted at 95° C. for 4 hours. They are then cooled to room temperature, and after concentration, separation and purification by silica gel column chromatography is carried out yielding title compound 12D, a yellow solid.

Compound 12D is dissolved in formic acid, and reacted for 4 hours at 35° C. A saturated potassium carbonate solution is used to adjust the reaction system to alkaline, and ethyl acetate is used for extraction, then drying with anhydrous sodium sulphate, filtration and concentration carried out, yielding the title compound 12E, a pale yellow oily substance.

Compound 12E is dissolved in DMF and INT-3, HATU and DIPEA added, then reacted overnight at room temperature after addition is complete. Water is added to the system, then ethyl acetate is used for extraction, drying with sodium sulphate takes place, and after filtration and concentration, separation and purification by silica gel column chromatography is carried out yielding the title compound 12F, a yellow solid.

Compound 12F is dissolved in formic acid, and reacted at 35° C. for 4 hours. A saturated solution of potassium carbonate is used to adjust the reaction system to slightly alkaline, ethyl acetate is used for extraction, anhydrous sodium sulphate used for drying, then after filtration and concentration, separation and purification by silica gel column chromatography is carried out yielding the title compound 12.

In one embodiment, (S)—N—((S)-2-(4-(benzo[d] thiazol-2-yl)-2-fluorophenyl)-1-cyanoethyl)-1,4-oxazepane-2-carboxamide (compound 13) is synthesized according to the following synthesis route.

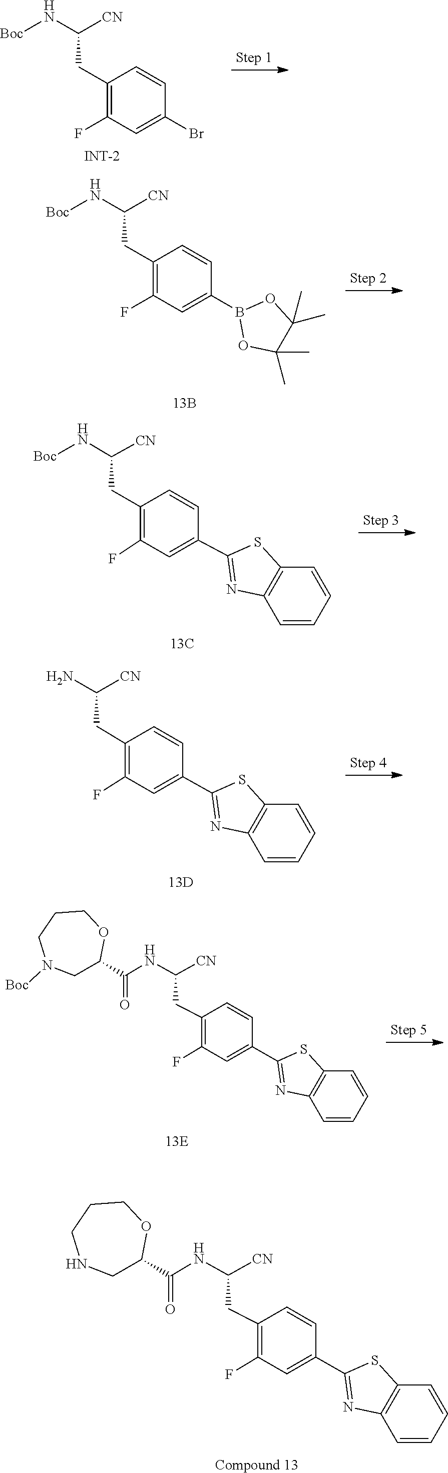

INT-2 is dissolved in 1,4-dioxane, bis(pinacolato)diboron, potassium acetate, and Pd (dppf) Cl₂·DCM are added, and then heated to 100° C. and are reacted for 2 hours after addition of protective nitrogen. After cooling to room temperature, filtration and concentration takes place, and the residue is subjected to separation and purification by silica gel column chromatography. This yields compound 13B as a colourless liquid.

Compound 13B is dissolved in 1,4-dioxane and water, 2-benzothiazole, Pd (dppf) Cl₂· DCM (0.17 g, 0.20 mmol) and potassium carbonate are added, and then reacted at 90° C. for 2 hours after protective nitrogen is added. Reaction takes place at room temperature overnight. After concentration until dry, a saturated aqueous solution of ammonium chloride is added, and ethyl acetate is used for extraction, after the organic phase is combined, anhydrous sodium sulphate is used for drying, and after filtration and concentration, the residue is subjected to separation and purification by silica gel column chromatography. This yields compound 13C as a pale yellow liquid.

Compound 13C is dissolved in formic acid, and then reacted at 30° C. for 3 hours. A saturated aqueous solution of sodium bicarbonate is dripped in to adjust pH to around 8, and ethyl acetate is used for extraction, the organic phase is combined, and then is dried with anhydrous sodium sulphate, filtration and concentration is then carried out. This yields compound 13D as a colourless liquid.

Compound 13D is dissolved in dichloromethane, and INT-1, diisopropylethylamine and HATU are added, then these are reacted overnight at room temperature. A saturated aqueous solution of sodium chloride is added, and ethyl acetate is used for extraction, the organic phase is washed with a saturated aqueous solution of sodium chloride, and is dried with anhydrous sodium sulphate. The product is filtered, concentrated, and the residue is subjected to separation and purification by silica gel column chromatography yielding the compound 13E, as a pale yellow solid.

Compound 13E is dissolved in formic acid, and is reacted for 4 hours at 35° C. Concentration until dry is carried out, and ethyl acetate is added, a saturated aqueous solution of sodium bicarbonate is added to adjust the pH to about 8, and the organic layer separated, ethyl acetate is used for extraction, and the organic phase combined, anhydrous sodium sulphate is used for drying, and filtration and concentration is carried out, and the residue is subjected to separation and purification by silica gel column chromatography, and this yields compound 13.

In one embodiment, (S)—N—((S)-1-cyano-2-(3-fluoro-4′-((4-methylpiperazin-1-yl)methyl)-[1,1′-biphenyl]-4-yl)ethyl)-1,4-oxazepane-2-carboxamide (compound 14) is synthesized according to the following synthesis route.

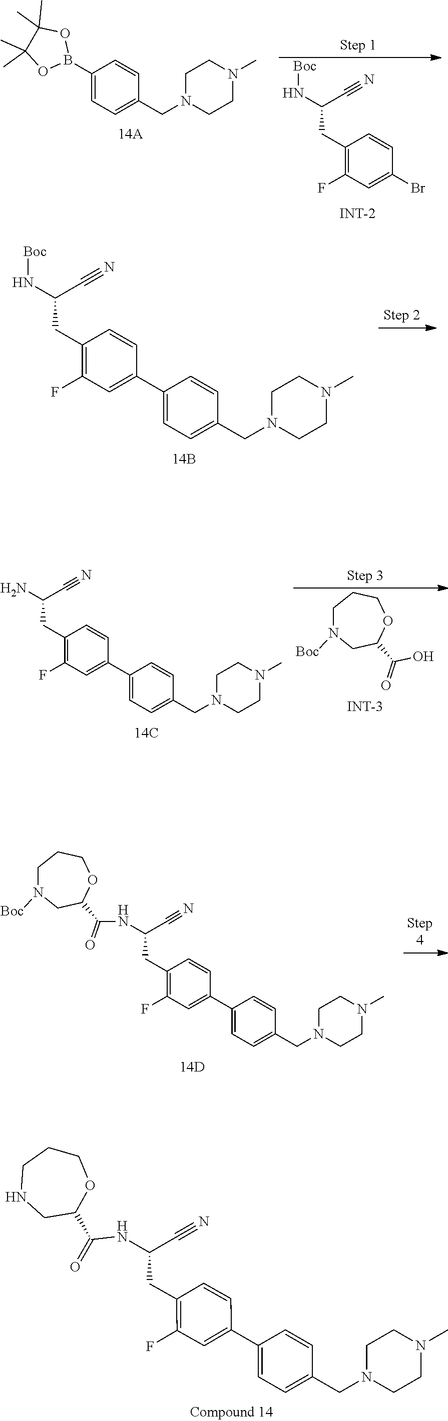

Compound 14A, INT-2, potassium carbonate and Pd (dppf) Cl₂ are dissolved in 1,4-dioxane and water, the atmosphere is replaced with nitrogen 3 times, the temperature raised to 95° C. and refluxing is carried out for 3 hours. Then filtration, and ethyl acetate are used to wash the filter cake. The filtrate is dried with anhydrous sodium sulphate, and is concentrated, and the residue that is obtained is subjected to separation and purification with silica gel column chromatography. This yields compound 14B, as a brown solid.

Compound 14B is dissolved in formic acid, and is stirred overnight at room temperature. Water and dichloromethane are added, and sodium bicarbonate is used to adjust pH to alkaline. The organic phase is separated, and the aqueous phase is subjected to extraction with dichloromethane. The organic phase is combined, and is then dried with anhydrous sodium sulphate, and is concentrated. The residue that is obtained is subjected to separation and purification with silica gel column chromatography. This yields compound 14C, as a brown oil substance.

Compound 14C, INT-3, HATU, and DIPEA are dissolved in dichloromethane, are stirred at room temperature for 4 hours, and a saturated sodium bicarbonate solution is used to wash the reaction liquid. The organic phase is separated, and anhydrous sodium sulphate is used for drying. Then, concentration is carried out, after which, the residue obtained is subjected to separation and purification with silica gel column chromatography. This yields compound 14D as a yellow solid.

Compound 14D and 2,6-lutidine are dissolved in dichloromethane, and are stirred in an ice bath. TBDMS triflate is dripped in to the flask, and after addition it is removed from the ice bath, and stirred at room temperature and reacted for 2 hours, a saturated sodium bicarbonate solution is used to wash the reaction liquid, and the organic phase is separated. Anhydrous sodium sulphate is used for drying and concentration is carried out. Then, after concentration, a residue is obtained and is subjected to separation and purification with silica gel column chromatography. This yields compound 14.

In one embodiment, (S)—N—((S)-1-cyano-2-(2-fluoro-4-(4-methylthiazol-2-yl)phenyl)ethyl)-1,4-oxazepane-2-carboxamide (compound 15) is synthesized according to the following synthesis route.

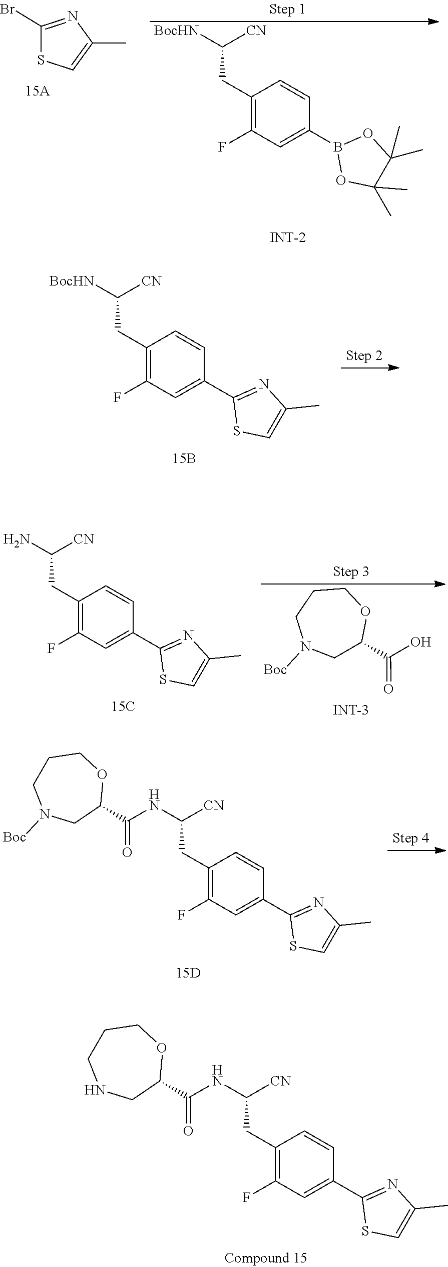

Compound 15A and INT-2, Pd (dppf) Cl₂, and potassium carbonate are dissolved in a mixed solution of dioxane and water, then are reacted under protective nitrogen at 90° C. for 5 hours. The reaction is cooled to room temperature, and water is added. Ethyl acetate is then used for extraction. The organic layer is combined, and saturated sodium bicarbonate and saturated domestic salt water are used for washing sequentially, anhydrous sodium sulphate is used for drying, the reaction is concentrated and then after concentration, the residue is subject to separation and purification with silica gel column chromatography. This yields compound 15B.

Compound 15B is dissolved in anhydrous formic acid, and is reacted at 50° C. for 20 minutes. The reaction is cooled to room temperature, and is concentrated to remove the majority of the solvent. A saturated sodium bicarbonate solution is added to the residue. Ethyl acetate is used for extraction, the organic layer is combined, anhydrous sodium sulphate is used for drying, and the residue is concentrated. Then, after concentration, the residue is subject to silica gel column chromatography separation and purification, and this yields compound 15C.

INT-3 is dissolved in DMF, and HATU and DIPEA are added under protective nitrogen and are stirred. Compound 15C is added, and is reacted at room temperature for 1 hour. Water is added to the reaction, and ethyl acetate is used for extraction, and the organic layer is combined, and anhydrous sodium sulphate is used for drying. Concentration is then carried out. After concentration, the residue is subject to silica gel column chromatography separation and purification. This yields compound 15D.

Compound 15D is dissolved in formic acid, and reacted for 30 minutes at 50° C. Concentration until dry is carried out, and ethyl acetate is added, a saturated aqueous solution of sodium bicarbonate is added to adjust the pH to about 8, and the organic layer separated, ethyl acetate is used for extraction, and the organic phase is combined, anhydrous sodium sulphate is used for drying, and filtration and concentration is then carried out, and the residue is subject to separation and purification by silica gel column chromatography. This yields compound 15.

In one embodiment, (S)—N—((S)-2-(4-(1-acetylindolin-5-yl)-2-fluorophenyl)-1-cyanoethyl)-1,4-oxazepane-2-carboxamide (compound 16) is synthesized according to the following synthesis route.

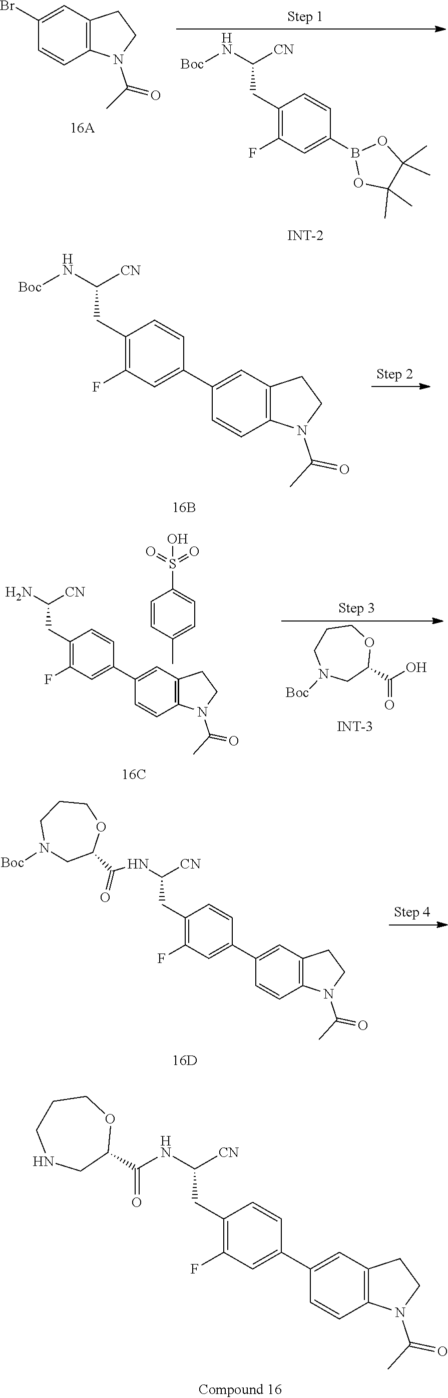

Compound 16A, INT-2, Pd (dppf) Cl₂· DCM and potassium carbonate are dissolved in 1,4-dioxane. Water is added and then nitrogen is used to replace the atmosphere 3 times, then these react under nitrogen for 4 hours at 90° C. The reaction liquid is concentrated until dry, and dichloromethane is added to dissolve, then filtration and concentration takes place. The residue is then subject to separation and purification by silica gel column chromatography and this yields the title compound 16B as a white solid.

Compound 16B is dissolved in acetonitrile, p-toluenesulfonic acid monohydrate is added, and these react at room temperature for 16 hours, and then filtered. The filter cake is rinsed once with acetonitrile, and the filter cake is centrifuged dry. This yields compound 16C.

Compound INT-3 is dissolved in dichloromethane, then triethylamine and HATU are added, then reacted while stirring at room temperature for 1 hour, then compound 16C is added, and is left at room temperature overnight. After the reaction finishes, concentration is carried out, and the raw product 16D is obtained.

Compound 16D is dissolved in acetonitrile, and p-toluenesulfonic acid monohydrate is added. The mixture reacts at 16 hours at room temperature, is concentrated until dry, and ethyl acetate is added. Next, a saturated aqueous solution of sodium bicarbonate is dripped in to adjust pH to about 8. The organic layer is separated, and ethyl acetate is used for extraction. Organic phase is combined, and after combination, the organic phase dries using anhydrous sodium sulphate and is then subject to filtration and concentration. The resulting residue is purified yielding the title compound 16.

In one embodiment, (S)—N—((S)-1-cyano-2-(4′-cyano-3′-cyclopropyl-3-fluoro-[1,1′-biphenyl]-4-yl)ethyl)-1,4-oxazepane-2-carboxamide (compound 17) is synthesized according to the following synthesis route.

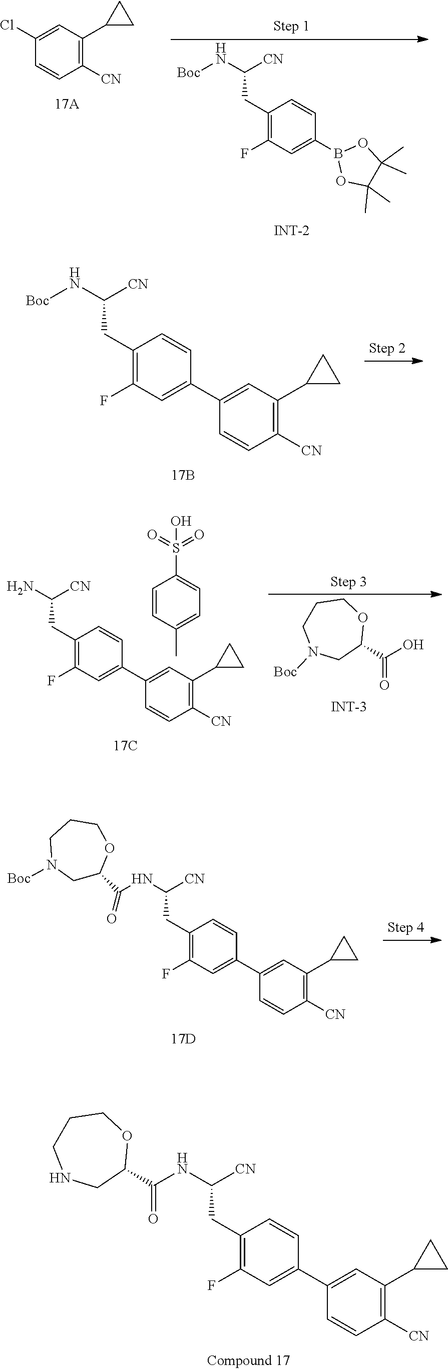

Compound 17A, INT-2, Pd (dppf) Cl₂· DCM and potassium carbonate are dissolved in 1,4-dioxane, water is added and then nitrogen is used to replace the atmosphere 3 times. The mixture reacts under nitrogen for 16 hours at 100° C. The reaction liquid is concentrated until dry, and dichloromethane is added to dissolve. Filtration and concentration is carried out, then the residue is subjected to separation and purification by silica gel column chromatography. This yields compound 17B as a white solid.

Compound 17B is dissolved in acetonitrile, p-toluenesulfonic acid monohydrate is added, and the mixture reacts at room temperature for 16 hours. Upon completion, the reaction is filtered, and the filter cake is then rinsed once with acetonitrile. The filter cake is centrifuged dry, and this yields compound 17C as a white solid.

INT-3 is dissolved in dichloromethane, then triethylamine and HATU are added. The mixture is reacted while stirring at room temperature for 1 hour. Compound 17C is added, and left at room temperature overnight. After the reaction finishes, concentration is carried out, and the product 17D is used directly in the next reaction step.

The product 17D is dissolved in acetonitrile, and p-toluenesulfonic acid monohydrate are added, then are reacted for 16 hours at room temperature, the mixture is concentrated until dry, and ethyl acetate is added, a saturated aqueous solution of sodium bicarbonate is dripped in to adjust pH to about 8, the organic layer is separated, and ethyl acetate is used for extraction. After combination, the organic phase is dried using anhydrous sodium sulphate, is filtered and concentrated, and the residue is subjected to separation and purification by silica gel column chromatography. This yields the title compound 17.

In one embodiment, (S)—N—((S)-1-cyano-2-(2-fluoro-4-(3-oxoisoindolin-5-yl)phenyl)ethyl)-1,4-oxazepane-2-carboxamide (compound 30) is synthesized according to the following synthesis route.

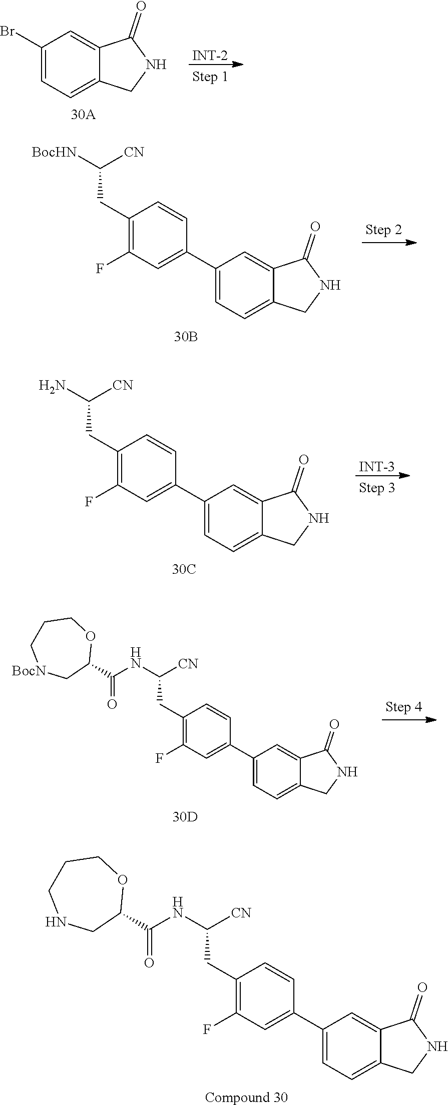

Compound 30A, INT-2, Pd (dppf) C₁₂ and potassium carbonate are added sequentially to 1,4-dioxane and water. The system atmosphere is replaced 3 times with nitrogen. The mixture is then reacted at 100° C. for 2.5 hours. After the reaction ends, it is cooled to room temperature, and water is added. The aqueous phase is extracted using ethyl acetate, the organic phase is combined and a saturated aqueous solution of sodium chloride is used for washing. Drying with anhydrous sodium sulphate and filtration take place and the filtrate is subjected to vacuum concentration. The residue is separated using silica gel column chromatography and this yields compound 30B.

Compound 30B is dissolved in formic acid, then is reacted at 50° C. for 10 minutes, is concentrated until dry. Ethyl acetate is then added, a saturated aqueous solution of sodium bicarbonate is dripped in to adjust pH to about 8, the organic layer is separated, and the organic phases extracted with ethyl acetate. After the organic phase is combined, anhydrous sodium sulphate is used for drying, then filtration and concentration is carried out and this yields compound 30C.

Compound 30C is dissolved in N,N-dimethylformamide, and INT-3, triethylamine and HATU are added, after which they react for 1 hour at room temperature. A saturated aqueous solution of sodium chloride is added, and ethyl acetate is used for extraction, the organic phase is washed using a saturated aqueous solution of sodium chloride, then is dried with anhydrous sodium sulphate, filtered, concentrated, and the residue is separated with silica gel column chromatography. This yields compound 30D.

Compound 30D is dissolved in formic acid, and reacted for 10 minutes at 50° C. Vacuum concentration is carried out, and ethyl acetate is added, then a saturated aqueous solution of sodium bicarbonate is used to adjust pH to about 8, the organic layer is separated, and dichloromethane is used for extraction, then the organic phase is combined and anhydrous sodium sulphate is used for drying, then after filtering and concentrating, the residue is subjected to separation and purification by silica gel column chromatography and this yields compound 30.

In one embodiment, (S)—N—((S)-1-cyano-2-(2-fluoro-4-(2-methyl-3-oxoisoindolin-5-yl)phenyl)ethyl)-1,4-oxazepane-2-carboxamide (compound 31) is synthesized according to the following synthesis route.

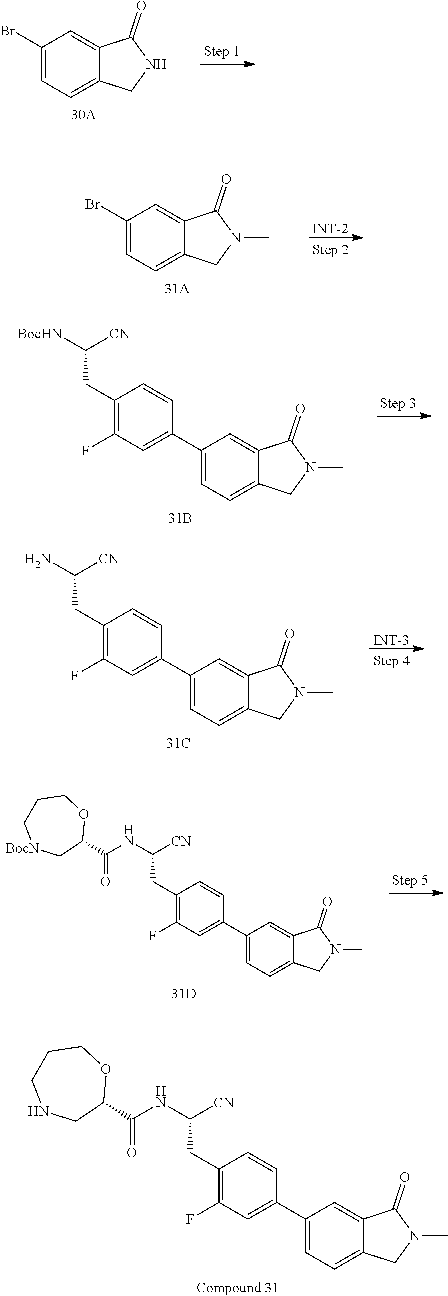

Compound 30A is dissolved in dry N,N-dimethylformamide, cooled to 0° C. under protective nitrogen, and sodium hydride is added in batches; after addition is complete, the mixture reacts for 20 minutes, and methyl iodide is dripped into the system. After addition of the methyl iodide is complete, the reaction takes place at room temperature for 30 minutes. Water is added to quench the reaction, and ethyl acetate is used for extraction, then the organic phase is combined, and is washed with saturated domestic salt water, is dried with anhydrous sodium sulphate, is filtered and the filtrate vacuum is concentrated, and the residue is subjected to silica gel column chromatography separation. This yields compound 31A.

Compound 31A, INT-2, Pd (dppf) C₁₂ and potassium carbonate are added sequentially to 1,4-dioxane and water, the system atmosphere is replaced three times with nitrogen, then they are reacted at 100° C. for 2 hours. After the reaction ends they are cooled to room temperature, water added, and the aqueous phase is extracted using ethyl acetate, the organic phase is combined and a saturated aqueous solution of sodium chloride is used for washing, then drying with anhydrous sodium sulphate and filtration take place, and the filtrate is subjected to vacuum concentration. The residue is separated using silica gel column chromatography and this yields compound 31B.

Compound 31B is dissolved in formic acid, then reacted at 50° C. for 10 minutes, is concentrated till dry, and ethyl acetate is added. A saturated aqueous solution of sodium bicarbonate is dripped in to adjust pH to around 8, the organic layer is separated, and the organic phase is extracted with ethyl acetate, after the organic phase is combined, anhydrous sodium sulphate is used for drying, then filtration and concentration are carried out and this yields compound 31C.

Compound 31C is dissolved in N,N-dimethylformamide, and INT-3, triethylamine and HATU are added, after which they are reacted for 1 hour at room temperature. A saturated aqueous solution of sodium chloride is added, and ethyl acetate is used for extraction, the organic phase is washed using a saturated aqueous solution of sodium chloride, then is dried with anhydrous sodium sulphate, filtered, concentrated, and the residue is separated with silica gel column chromatography. This yields compound 31D.

Compound 31D is dissolved in formic acid, and reacts for 10 minutes at 50° C. Vacuum concentration is carried out, and ethyl acetate is added. Then a saturated aqueous solution of sodium bicarbonate is used to adjust pH to about 8, the organic layer is separated, and dichloromethane is used for extraction, then the organic phase is combined and anhydrous sodium sulphate is used for drying. Then, after filtering and concentration, the residue is subjected to separation and purification by silica gel column chromatography and this yields compound 31.

In one embodiment, (S)—N—((S)-1-cyano-2-(5-(3-methyl-2-oxo-2,3-dihydrobenzo[d] oxazol-5-yl)pyridin-2-yl)ethyl)-1,4-oxazepane-2-carboxamide and(S)—N—((R)-1-cyano-2-(5-(3-methyl-2-oxo-2,3-dihydrobenzo[d] oxazol-5-yl)pyridin-2-yl)ethyl)-1,4-oxazepane-2-carboxamide (compound 33 and compound 34) is synthesized according to the following synthesis route.

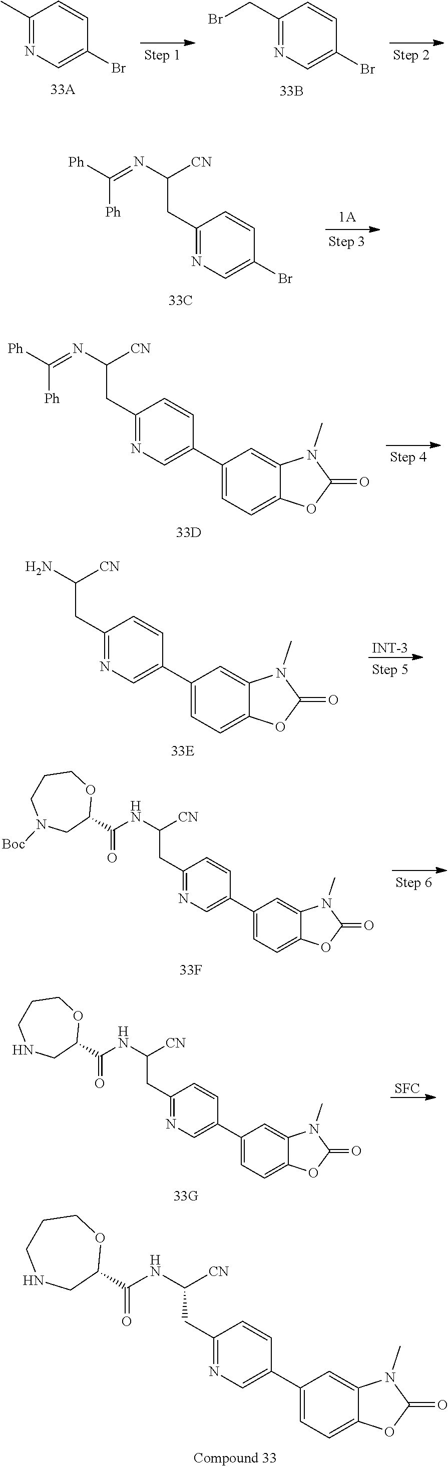

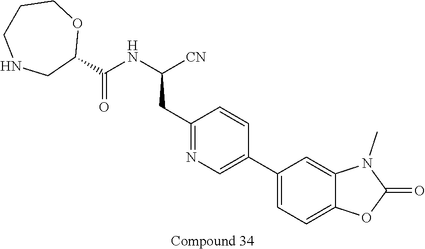 [SMILES: Cn1c(=O)oc2ccc(-c3ccc(C[C@H](C#N)NC(=O)[C@@H]4CNCCCO4)nc3)cc21 ; "Compound 34"]

5-bromo-2-picoline (compound 33A) is dissolved in tetrachloromethane, and after N-bromosuccinimide and azodiisobutyronitrile are added, they are reacted at 90° C. for 2.5 hours. The product is cooled to room temperature, and the reaction liquid is concentrated, and the residue is subjected to separation and purification yielding the compound 33B.

Compound 33B and N-(diphenylmethylene)aminoacetonitrile are dissolved in dichloromethane, and benzyltrimethylammonium chloride is added. Next, an aqueous solution of sodium hydroxide is added while stirring vigorously, and a reaction takes place overnight at room temperature. Water is added, and dichloromethane is used for extraction, the organic layer combined, and anhydrous sodium sulphate is used for drying, then after concentration the residue is subjected to separation and purification, e.g., with silica gel column chromatography

Compound 33C, compound 1A, Pd (dppf) Cl₂ and potassium carbonate are dissolved in a mixed solution of dioxane and water, then reacted under protective nitrogen at 90° C. for 5 hours. The product is cooled to room temperature, water is added, and ethyl acetate is used for extraction, the organic layer combined, and saturated sodium bicarbonate and saturated domestic salt water are used for washing sequentially, anhydrous sodium sulphate is used for drying, then after concentration, the residue is subjected to separation and purification, e.g., with silica gel column chromatography.

Compound 33D is then dissolved in tetrahydrofuran and water (5 ml), 2.5 ml of a 1M aqueous solution of HCl is dripped in, and then a reaction takes place at room temperature for 5 hours. Diethyl ether (15 ml×3) is used for extraction of the reaction fluid and it is discarded, the pH of the aqueous layer is adjusted to around 12, and DCM is used for extraction. The organic layer is combined, anhydrous sodium sulphate is used for drying, and compound 33E is obtained after concentration, without further purification, and is used directly in the next step.

Compound 33E is dissolved in dichloromethane, and DIPEA, HATU and intermediate INT-3 are added sequentially, and reacted at room temperature for 1 hour. Once the reaction is complete, water is poured into the reaction liquid, and stratification occurs, and the organic phase washed sequentially with water and saturated domestic salt water, dried with anhydrous sodium sulphate, and the raw product with a yellow oily appearance is obtained by vacuum concentration. The raw product undergoes purification, yielding 33F, a white solid.

Compound 33F is dissolved in dichloromethane, and TMSOTf is added, and 2,6-lutedine is dripped in gradually using an ice bath, and after this the temperature is raised to room temperature and a reaction takes place for 1 hour. The reaction liquid is poured into a saturated solution of ammonium chloride, and dichloromethane is used for extraction. The organic phase is washed with saturated sodium chloride, and anhydrous sodium sulphate is used for drying, the product is concentrated, and then undergoes purification, e.g., via column chromatography purification and separation, yielding compound 33G.

Supercritical fluid chromatography (SFC) chiral separation is carried out on compound 33G, yielding compounds 33 and 34.

In one embodiment, N—((S)-1-cyano-2-(3-fluoro-4′-(pentafluoro-16-sulfanyl)-[1,1′-biphenyl]-4-yl)ethyl)-6-methoxy-1,4-oxazepane-2-carboxamide (compound 43) is synthesized according to the scheme below.

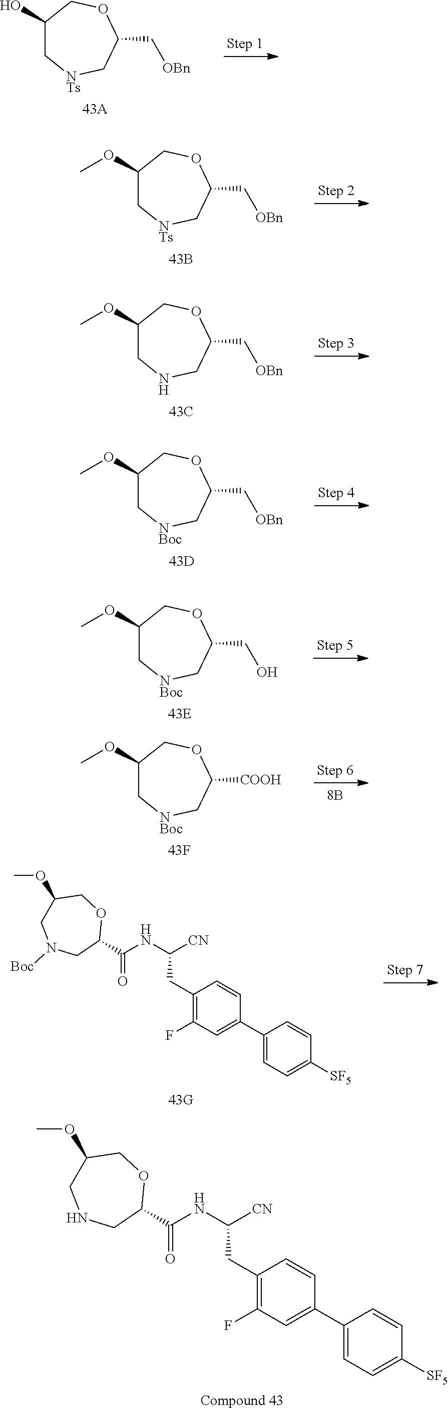

Compound 43A (prepared according to Eur. J. Org. Chem. 2007, 2107-2113 DOI: 10.1002/ejoc.200700011) is dissolved in tetrahydrofuran at 0° C. Sodium hydride is then added and the mixture is stirred for 30 minutes. Methyl iodide is then added and the reaction is carried out at room temperature for 3-4 hours. Then at 0° C., water is added, ethyl acetate (EA) is then used for extraction, and saturated domestic salt water is used for washing, anhydrous sodium sulphate is used for drying, then after concentration, 43B, a colourless oily substance, is obtained.

Compound 43B is dissolved in methanol, magnesium filings are added, and the mixture is subjected to ultrasound for 2 hours at 50° C., and then is reacted at room temperature for 16 hours. The product is then filtered and concentrated, yielding 43C, a white oily substance, and this used directly in the next step.

Compound 43C is dissolved in DCM, and triethylamine (0.087 g, 0.85 mmol) and TBSCI (0.37 g, 1.71 mmol) added sequentially and reacted for 2 hours at room temperature under protective nitrogen, then concentrated till dry, and subjected to column chromatography purification (PE:EA=10:1-4:1), yielding 43D, a colourless oil substance (0.12 g, yield 47%).

Compound 43D is dissolved in methanol, and Pd/C is added, and the mixture is reacted in a hydrogen atmosphere for 24 hours, filtered, and is concentrated until dry, which then yields compound 43E, a colourless oily substance.

43E is dissolved in acetone. Then saturated sodium bicarbonate, sodium bromide, TEMPO are added, and trichloroisocyanuric acid is added at 0° C., and a reaction takes place for 16 hours, and dilute hydrochloric acid added to adjust pH to 5-6, and water added, dichloromethane is used for extraction, washing in water is carried out, and washing with saturated sodium chloride is carried out. Anhydrous sodium sulphate is used for drying, and then after concentration, 43F, a yellow oil substance, is obtained.

Compound 8B, compound 43F, HATU and DIEA are mixed with and dissolved in DMF, and stirred overnight. When the reaction is complete, water is added, EA is used for extraction. The organic phase is then dried and concentrated, and column chromatography separation is carried out, yielding compound 43G.

Compound 43g is dissolved in acetonitrile. p-toluenesulfonic acid is then added, and the mixture is heated to 40° C., and reacted for 2 hours. After the reaction is complete, the reaction liquid is concentrated, EA and a saturated aqueous solution of sodium bicarbonate is added for extraction, the liquid is separated, and the organic phase is dried and concentrated, and separated by column chromatography, yielding the title compound 43.

### Example 2—Preparation of Certain Intermediates

For examples 2-5, 1H NMR and LC/MS analysis were carried out as follows, unless indicated otherwise.

¹H NMR analysis:

¹H-NMR spectra were recorded on a Bruker Ultrashield (400 MHz). The multiplicity of a signal is designated by the following abbreviations: s, singlet; d, doublet; t, triplet; q, quartet; dd, doublet of doublets; dt, doublet of triplets; m, multiplet.

All observed coupling constants, J, are reported in Hertz (Hz).

Exchangeable protons are not always observed.

LC/MS analysis:

LC/MS Method AN01_001_012:

LC-MS data was generated using a Waters Acquity system: TUV detector, SQD2 MS detector, Sedere SEDEX 80 (light scattering detector).

LC-MS method: reverse phase HPLC analysis

Column Agilent: Cortecs C18

Solvent A: Water with Formic Acid (0.1% V/V)

Solvent B: Acetonitrile

Gradient table:

| Time (min) | Flow (mL/min) | % A | % B | Curve |
|---|---|---|---|---|
| 0 | 0.7 | 98 | 2 | initial |
| 2 | 0.7 | 0 | 100 | Linear |
| 2.5 | 0.7 | 0 | 100 | Linear |
| 2.6 | 0.7 | 98 | 2 | Linear |
| 5 | 0.7 | 98 | 2 | Linear |

LC/MS Method AN01_001_026:

LC-MS data was generated using a Waters Acquity system: TUV detector, SQD2 MS detector, Sedere SEDEX 80 (light scattering detector).

LC-MS method: reverse phase HPLC analysis

Column Agilent: Poroshell

Solvent A: Water with Formic Acid (0.1% V/V)

Solvent B: Acetonitrile

Gradient table:

| Time (min) | Flow (mL/min) | % A | % B | Curve |
|---|---|---|---|---|
| 0 | 1 | 98 | 2 | initial |
| 2 | 1 | 98 | 2 | 6 |
| 12 | 1 | 0 | 100 | 6 |
| 15.4 | 1 | 0 | 100 | 6 |
| 16.7 | 1 | 98 | 2 | 6 |
| 18.4 | 1 | 98 | 2 | 6 |

UV detection: 220 nm

Chiral SFC purity analysis conditions:

Column Details: ChiralPak OD-3 (4.6×100 mm)

Column Temperature: 35° C.

Flow Rate: 3.5 mL/min

Detector Wavelength: 220-410 nm

Injection Volume: 2 μL

BPR: 1500PSI

Isocratic Conditions: i-PrOH: CO₂, 30:70

### Synthetic Scheme for the Preparation of Intermediates B1-2-4 & B1-2-6

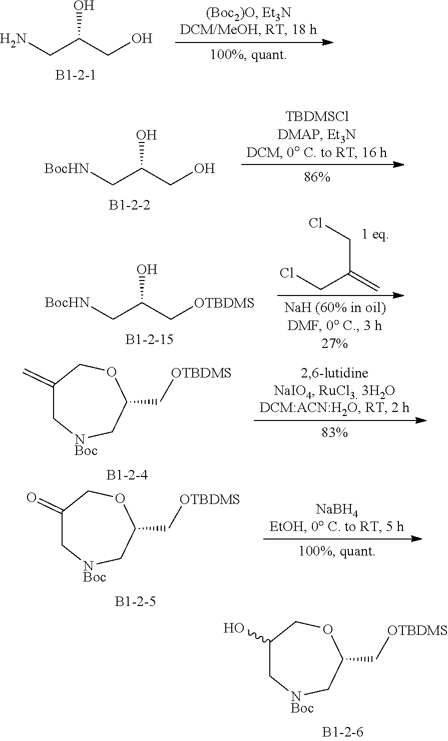

### Experimental Procedure

### Tert-Butyl N-[(2S)-2,3-Dihydroxypropyl]Carbamate B1-2-2

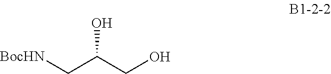 [SMILES: CC(C)(C)OC(=O)NC[C@H](O)CO]

To a solution of (2S)-3-aminopropane-1,2-diol (1 eq., 14.4 g, 158.1 mmol) and Et₃N (1.01 eq., 22.2 mL, 159.6 mmol) in anhydrous MeOH (245 mL) was added a solution of Boc₂O (1.2 eq., 41.4 g, 189.7 mmol) in anhydrous DCM (41 mL) at room temperature under argon atmosphere. The reaction mixture was stirred at room temperature for 18 h and then concentrated under reduced pressure to afford B1-2-2 as a pale yellow oil (30.2 g, quant.). The crude was considered quantitative and used as such.

LC/MS (AN01_001_012): Rt=1.69 min, non-UV active, [M+Na]⁺=214.1. concentrated under reduced pressure to afford B1-2-2 as a pale yellow oil (30.2 g, quant.). The crude was considered quantitative and used as such.

LC/MS (AN01_001_012): Rt=1.69 min, non-UV active, [M+Na]⁺=214.1.

### tert-butyl N-[(2S)-3-[(tert-butyldimethylsilyl)oxy]-2-hydroxypropyl]carbamate B1-2-15

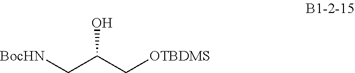 [SMILES: CC(C)(C)OC(=O)NC[C@H](O)CO[Si](C)(C)C(C)(C)C]

To a solution of B1-2-2 (1 eq., 10.0 g, 47.6 mmol) and Et₃N (1.2 eq., 7.94 mL, 57.1 mmol) in anhydrous DCM (76 mL) were added TBDMSCI (1.1 eq., 7.90 g, 52.4 mmol) and DMAP (0.05 eq., 0.290 g, 2.38 mmol) at 0° C. under argon atmosphere. The reaction mixture was allowed to warm to room temperature and stirred for 16 h. The resulting mixture was diluted with DCM (50 mL) and water (150 mL) and the two layers were separated. The aqueous layer was extracted with DCM (2×50 mL) and the combined organic layers were dried over Na₂SO₄, filtered and concentrated under reduced pressure. The crude residue was purified by silica gel flash chromatography (220 g, gradient: Cyclohexane/EtOAc from 100:0 to 65:35) to afford B1-2-15 as a colorless oil (12.4 g, 86%).

LC/MS (AN01_001_012): Rt=2.68 min, non-UV active, [M-C₄H₈+H]⁺=250.2.

### tert-butyl(2S)-2-{[(tert-butyldimethylsilyl)oxy] methyl}-6-methylidene-1,4-oxazepane-₄-carboxylate B1-2-4

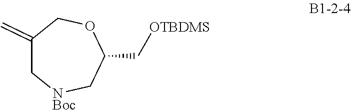 [SMILES: C=C1CO[C@H](CO[Si](C)(C)C(C)(C)C)CN(C(=O)OC(C)(C)C)C1]

To a suspension of NaH 60% in oil (2.1 eq., 1.65 g, 41.3 mmol) in anhydrous DMF (35 mL) was added 3-chloro-2-chloromethyl-1-propene (1 eq., 2.27 mL, 19.6 mmol) at 0° C. under argon atmosphere. The reaction mixture was stirred at 0° C. for 10 min before the dropwise addition of a solution of B1-2-15 (1 eq., 6.00 g, 19.6 mmol) in THF (24 mL). The resulting mixture was allowed to warm to room temperature and stirred for 3 h. The reaction mixture was diluted with water (150 mL) and extracted with Et₂O (3×50 mL). The combined organic layers were washed with brine (150 mL), dried over Na₂SO₄, filtered and concentrated under reduced pressure. The crude residue was purified by silica gel flash chromatography (220 g, gradient: Cyclohexane/EtOAc from 100:0 to 91:9) to afford B1-2-4 as a colorless oil (1.90 g, 27%).

LC/MS (AN01_001_012): Rt=3.17 min, 88%, [M-C₅H₈O₂+H]⁺=258.2.

### tert-butyl(2S)-2-{[(tert-butyldimethylsilyl)oxy] methyl}-6-oxo-1,4-oxazepane-4-carboxylate B1-2-5

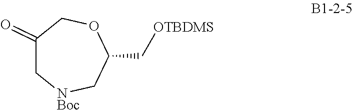 [SMILES: CC(C)(C)OC(=O)N1CC(=O)CO[C@H](CO[Si](C)(C)C(C)(C)C)C1]

To a solution of B1-2-4 (1 eq., 880 mg, 2.46 mmol) in a mixture of DCM (15 mL) and acetonitrile (15 mL) were added successively 2,6-lutidine (2 eq., 0.570 mL, 4.92 mmol), water (22 mL) and sodium periodate (4 eq., 2.10 g, 9.84 mmol) at room temperature. A solution of RuCl₃·3H₂O (0.035 eq., 22.5 mg, 0.086 mmol) in water (2.5 mL) was then added dropwise and the resulting brown suspension was stirred vigorously at room temperature for 2 h. The reaction mixture was diluted with water (100 mL) and extracted with DCM (3×100 mL). The combined organic layers were washed with brine (100 mL), dried over Na₂SO₄, filtered and concentrated under reduced pressure. The crude residue was purified by silica gel flash chromatography (40 g, gradient: Cyclohexane/EtOAc from 100:0 to 90:10) to afford B1-2-5 as a colorless oil (737 mg, 83%).

LC/MS (AN01_001_012): Rt=3.02 min, 100%, [M-C₄H₈+H]⁺=304.1.

### tert-butyl(2S)-2-{[(tert-butyldimethylsilyl)oxy] methyl}-6-hydroxy-1,4-oxazepane-4-carboxylate B1-2-6

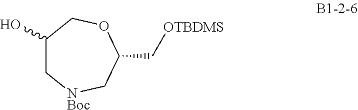 [SMILES: CC(C)(C)OC(=O)N1CC(O)CO[C@H](CO[Si](C)(C)C(C)(C)C)C1]

To a solution of B1-2-5 (1 eq., 730 mg, 2.03 mmol) in absolute anhydrous EtOH (21 mL) was added NaBH₄ (2 eq., 154 mg, 4.06 mmol) at 0° C. under argon atmosphere. The resulting solution was allowed to warm to room temperature and stirred for 5 h. The reaction mixture was quenched at room temperature with a saturated aqueous NH₄Cl solution (20 mL) and then diluted with water (100 mL) and DCM (100 mL). The two layers were separated and the aqueous layer was extracted with DCM (2×100 mL). The combined organic layers were dried over Na₂SO₄, filtered and concentrated under reduced pressure to afford B1-2-6 as a colorless oil (734 mg, 100%, diastereomeric mixture).

LC/MS (AN01_001_012): Rt=2.80 min, non-UV active, [M-C₄H₈+H]⁺=306.2.

### Example 2—Synthesis of (2S,6S)—N—((S)-1-cyano-2-(4-(3-methyl-2-oxo-2,3-dihydrobenzo[d] oxazol-5-yl)phenyl)ethyl)-6-hydroxy-1,4-oxazepane-2-carboxamide (INSM-201)

INSM-201 was synthesized as follows.

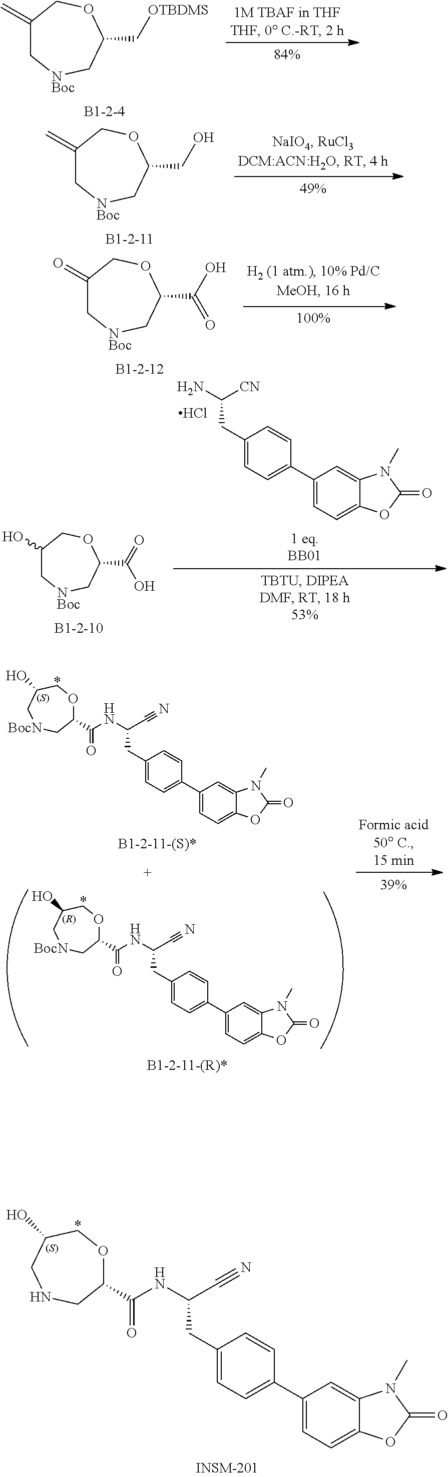

### Experimental Procedure

### tert-butyl(2S)-2-(hydroxymethyl)-6-methylidene-1,4-oxazepane-4-carboxylate B1-2-11

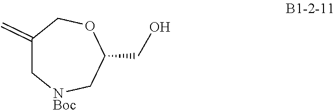 [SMILES: C=C1CO[C@H](CO)CN(C(=O)OC(C)(C)C)C1]

Starting from B1-2-4 (1 eq., 1.90 g, 5.31 mmol) and using general procedure A, B1-2-11 was obtained as a colorless oil (1.09 g, 84%) after purification by silica gel flash chromatography (25 g, gradient: Cyclohexane/EtOAc from 95:5 to 50:50).

LC/MS (AN01_001_012): Rt=2.04 min, 100%, [M+Na]=266.2.

### (2S)-4-[(tert-butoxy) carbonyl]-6-oxo-1,4-oxazepane-2-carboxylic acid B1-2-12

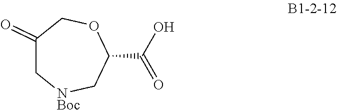 [SMILES: CC(C)(C)OC(=O)N1CC(=O)CO[C@H](C(=O)O)C1]

To a solution of B1-2-11 (1 eq., 440 mg, 1.81 mmol) in a mixture of DCM (5 mL), ACN (7.5 mL) and H₂O (4 mL) were added sodium periodate (5 eq., 1.93 g, 9.04 mmol) and RuCl₃ (0.2 eq. 75.0 mg, 0.36 mmol) at room temperature. The resulting mixture was vigorously stirred at room temperature for 4 h. The reaction mixture was diluted with DCM (20 mL) and a saturated aqueous solution of NaHCO₃ (20 mL). The two layers were separated, and the aqueous layer was washed with DCM (20 mL). The aqueous layer was then acidified with an aqueous solution of 3M HCl until pH ~1 and extracted with DCM (2×20 mL). The combined organic layers were dried over Na₂SO₄, filtered and concentrated under reduced pressure to afford B1-2-12 as a colorless oil (225 mg, 48%).

LC/MS (AN01_001_012): Rt=1.88 min, non-UV active [M−H]⁺=258.1.

### (2S)-4-[(tert-butoxy) carbonyl]-6-hydroxy-1,4-oxazepane-2-carboxylic acid B1-2-10

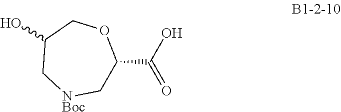 [SMILES: CC(C)(C)OC(=O)N1CC(O)CO[C@H](C(=O)O)C1]

To an argon purged solution of B1-2-12 (1 eq., 240 mg, 0.926 mmol) in MeOH (9.3 mL) was added 10% Pd/C (0.2 eq., 197 mg, 0.185 mmol) at room temperature. The resulting mixture was purged with argon (x3) and then with H₂ (3×). The reaction mixture was stirred under an atmospheric pressure of H₂ at room temperature for 18 h. The reaction mixture was purged with argon, filtered on a pad of celite and rinsed with MeOH (2×10 mL). The filtrate was concentrated under reduced pressure to afford B1-2-10 as a white solid (227 mg, 94%, diastereomeric mixture).

LC/MS (AN01_001_012): Rt=1.76 min, non-UV active, [M−H]⁺=260.1.

### tert-butyl (2S,6S*)-2-{[(1S)-1-cyano-2-[4-(3-methyl-2-oxo-2,3-dihydro-1,3-benzoxazol-5-yl)phenyl] ethyl]carbamoyl}-6 hydroxy-1,4-oxazepane-4-carboxylate B1-2-11-(S)* and tert-butyl(2S,6R*)-2-{[(1S)-1-cyano-2-[4-(3-methyl-2-oxo-2,3-dihydro-1,3-benzoxazol-5-yl)phenyl] ethyl]carbamoyl}-6-hydroxy-1,4-oxazepane-4-carboxylate B1-2-11-(R)*

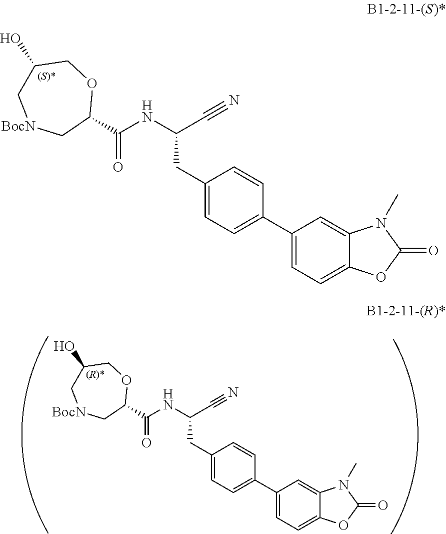

Starting from BB01 (1 eq., 264 mg, 0.802 mmol) and B1-2-10 (1.05 eq., 220 mg, 0.842 mmol), using general procedure B, the obtained diastereomeric mixture was separated by purification on silica gel flash chromatography (25 g, gradient: DCM/MeOH from 100:0 to 96:4) affording B1-2-11-(S)* (100 mg, 23%) and B1-2-11-(R)* (121 mg, 28%) as pale yellow solids. The stereochemistry(S)* was arbitrarily assigned to the first eluted product by flash chromatography and then the second eluted product was assigned (R)*.

B1-2-11-(S)*: LC/MS (AN01_001_012): Rt=2.30 min, 100%, [M-C₄H₈+H]⁺=481.2.

B1-2-11-(R)*: LC/MS (AN01_001_012): Rt=2.30 min, 100%, [M-C₄H₈+H]⁺=481.2.

### (2S,6S*)—N-[(1S)-1-cyano-2-[4-(3-methyl-2-oxo-2,3-dihydro-1,3-benzoxazol-5-yl)phenyl] ethyl]-6-hydroxy-1,4-oxazepane-2-carboxamide (INSM-201)

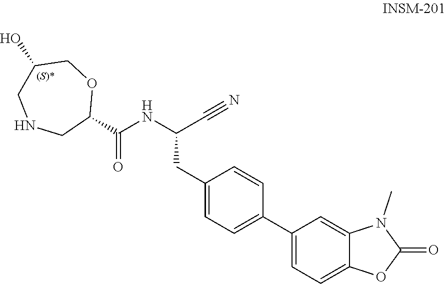 [SMILES: Cn1c(=O)oc2ccc(-c3ccc(C[C@@H](C#N)NC(=O)[C@@H]4CNC[C@H](O)CO4)cc3)cc21]

INSM-201 was prepared using general procedure C, starting from B1-2-11-(S)* (1 eq., 50.0 mg, 0.093 mmol). The crude residue was purified by preparative HPLC (gradient: H₂O (+0.1% TFA)/Acetonitrile, from 85:15 to 70:30, column: XBridge C18 (30×150 (5 μm)), Flow Rate: 43 mL/min). The collected fractions containing INSM-201 were combined and the organic solvent was removed under reduced pressure. The resulting aqueous layer was basified with solid NaHCO₃ until pH~8 and extracted with DCM (3×30 mL). The combined organic layers were dried over Na₂SO₄, filtered and concentrated under reduced pressure. The resulting residue was dissolved in a H₂O/MeOH mixture (90:10, 2 mL) and freeze-dried to afford INSM-201 as a white solid (15.7 mg, 39%).

LC/MS (AN01_001_026): Rt=6.44 min, 100%, [M+H]⁺=437.6.

¹H NMR (DMSO-d₆, 400 MHz): δ ppm 8.61 (d, J=8.4 Hz, 1H), 7.66 (d, J=8.3 Hz, 2H), 7.58-7.56 (m, 1H), 7.41-7.37 (m, 4H), 5.02 (q, J=8.0 Hz, 1H), 4.76 (d, J=5.3 Hz, 1H), 4.00 (dd, J=7.7, 4.1 Hz, 1H), 3.88 (dd, J=12.3, 4.8 Hz, 1H), 3.76-3.69 (m, 1H), 3.47 (dd, J=12.2, 7.8 Hz, 1H), 3.41 (s, 3H), 3.25-3.15 (m, 2H), 3.03 (dd, J=14.2, 4.0 Hz, 1H), 2.81 (dd, J=13.7, 3.8 Hz, 1H), 2.62 (dd, J=13.7, 5.9 Hz, 1H), 2.54-2.50 (m, 2H).

### Example 3 Syntheses of (2S)—N—((S)-1-cyano-2-(4-(3-methyl-2-oxo-2,3-dihydrobenzo[d]oxazol-5-yl)phenyl)ethyl)-6-methoxy-1,4-oxazepane-2- carboxamide (INSM-202)

INSM-202 was synthesized as follows.

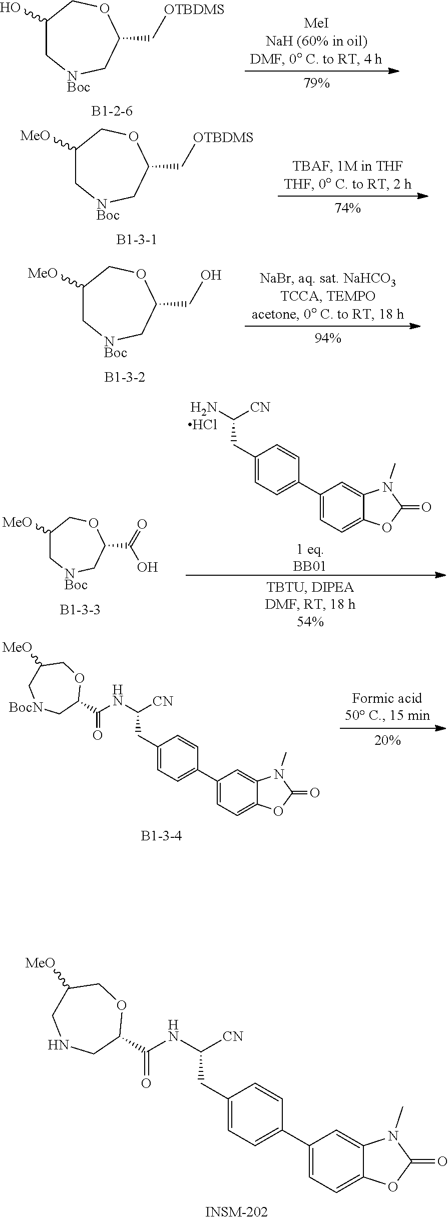

### Experimental Procedure

### tert-butyl(2S)-2-{[(tert-butyldimethylsilyl)oxy] methyl}-6-methoxy-1,4-oxazepane-4-carboxylate B1-3-1

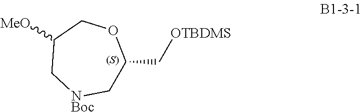

To a solution of B1-2-6 (1 eq., 500 mg, 1.38 mmol) and Mel (2 eq., 0.172 mL, 2.77 mmol) in anhydrous DMF (8 mL) was added 60% NaH on oil (1.1 eq., 60.8 mg, 1.52 mmol) at 0° C. under argon atmosphere. The resulting mixture was allowed to warm to room temperature and stirred for 4 h. The reaction mixture was quenched at room temperature with a saturated aqueous NH₄Cl solution (10 mL) and then diluted with water (50 mL) and EtOAc (50 mL). The two layers were separated and the aqueous layer was extracted with EtOAc (2×50 mL). The combined organic layers were dried over Na₂SO₄, filtered and concentrated under reduced pressure. The crude residue was purified by silica gel flash chromatography (25 g, gradient: Cyclohexane/EtOAc from 100:0 to 90:10) to afford B1-3-1 as a colorless oil (412 mg, 79%, diastereomeric mixture).

LC/MS (AN01_001_012): Rt=3.06 min, non-UV active, [M-C₄H₈+H]⁺=320.2.

### tert-butyl(2S)-2-(hydroxymethyl)-6-methoxy-1,4-oxazepane-4-carboxylate B1-3-2

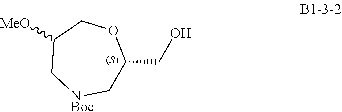

Starting from B1-3-1 (1 eq., 410 mg, 1.09 mmol) and using general procedure A, B1-3-₂ was obtained as a colorless oil (211 mg, 74%, diastereomeric mixture) after purification by silica gel flash chromatography (25 g, gradient: Cyclohexane/EtOAc from 100:0 to 25:75).

LC/MS (AN01_001_012): Rt=1.93 min, non-UV active, [M+Na]=284.2.

### 4-[(tert-butoxy) carbonyl]-6-methoxy-1,4-oxazepane-(25)-2-carboxylic acid B1-3-3

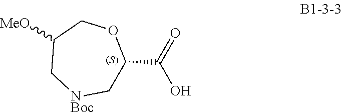

To a solution of B1-3-2 (1 eq., 210 mg, 0.804 mmol) in acetone (14 mL) were added successively sodium bromide (0.3 eq., 25.2 mg, 0.245 mmol) and a saturated aqueous NaHCO₃ solution (2 mL) at room temperature. To the resulting mixture were added trichlorocyanuric acid (2.2 eq., 411 mg, 1.77 mmol) and 2,2,6,6-tetramethylpiperidine-1-oxyl (0.03 eq., 3.76 mg, 0.0245 mmol) at 0° C. The reaction mixture was allowed to warm to room temperature and stirred for 18 h. Isopropanol (15 mL) was added at room temperature and the reaction mixture was stirred for 30 min. The reaction mixture was diluted with EtOAc (100 mL) and a saturated aqueous solution of NaHCO₃ (100 mL). The two layers were separated, and the aqueous layer was washed with EtOAc (100 mL). The aqueous layer was then acidified with an aqueous solution of 3M HCl until pH ~1 and extracted with DCM (2×100 mL). The combined organic layers were dried over Na₂SO₄, filtered and concentrated under reduced pressure to afford B1-₃-3 as a pale yellow oil (208 mg, 94%, diastereomeric mixture).

LC/MS (AN01_001_012): Rt=1.91 min, non-UV active, [M+Na]⁺=298.1.

### tert-butyl-(25)-2-{[(1S)-1-cyano-2-[4-(3-methyl-2-oxo-2,3-dihydro-1,3-benzoxazol-5-yl)phenyl] ethyl]-carbamoyl}-6-methoxy-1,4-oxazepane-4-carboxylate B1-3-4

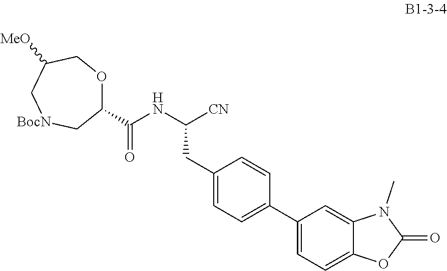 [SMILES: COC1CO[C@H](C(=O)N[C@H](C#N)Cc2ccc(-c3ccc4oc(=O)n(C)c4c3)cc2)CN(C(=O)OC(C)(C)C)C1]

Starting from BB01 (1 eq., 302 mg, 0.916 mmol) and B1-3-3 (1.05 eq., 265 mg, 0.962 mmol), using general procedure B, B1-3-4 was obtained as an orange solid (271 mg, 54%, diastereomeric mixture) after two purifications by silica gel flash chromatography (1st purification: 25 g, gradient: DCM/MeOH from 100:0 to 98:2; 2^{nd} purification: 25 g, gradient: cyclohexane/EtOAc from 100:0 to 40:60).

LC/MS (AN01_001_012): Rt=2.49 min, 100%, [M-C₄Hg+H]⁺=495.3.

### (6-rac)-(2S)—N-[(1S)-1-cyano-2-[4-(3-methyl-2-oxo-2,3-dihydro-1,3-benzoxazol-5-yl)phenyl] ethyl]-6-methoxy-1,4-oxazepane-2-carboxamide INSM-202

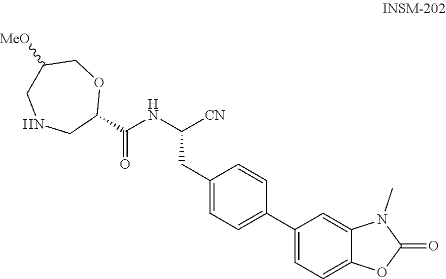 [SMILES: COC1CNC[C@@H](C(=O)N[C@H](C#N)Cc2ccc(-c3ccc4oc(=O)n(C)c4c3)cc2)OC1]

INSM-202 was prepared using general procedure C, starting from B1-3-4 (1 eq., 92.5 mg, 0.168 mmol). The crude residue was first purified by C18 reversed-phase flash chromatography (12g, gradient: H₂O (+0.1% TFA)/Acetonitrile, from 100:0 to 50:50). The collected fractions containing INSM-202 were combined and the organic solvent was removed under reduced pressure. The resulting aqueous layer was basified with solid NaHCO₃ until pH~8 and extracted with DCM (3×50 mL). The combined organic layers were dried over Na₂SO₄, filtered and concentrated under reduced pressure. The resulting residue was purified by preparative HPLC (gradient: H₂O (+0.1% TFA)/Acetonitrile, from 85:15 to 70:30, column: XBridge PFP (30×150 (5 μm)), Flow Rate: 43 mL/min). The collected fractions containing INSM-202 were combined and the organic solvent was removed under reduced pressure. The resulting aqueous layer was basified with solid NaHCO₃ until pH~8 and extracted with DCM (3×50 mL). The combined organic layers were dried over Na₂SO₄, filtered and concentrated under reduced pressure. The resulting residue was dissolved in a H₂O/MeOH mixture (90:10, 2 mL) and freeze-dried to afford INSM-202 as a white solid (15.3 mg, 20%, diastereomeric mixture, 1H NMR ratio: 4:6).

LC/MS (AN01_001_026): Rt=6.60 min, 99.9%, [M+H]⁺=451.6.

¹H NMR (DMSO-d₆, 400 MHz): δ ppm 8.64-8.61 (m, 1H), 7.67-7.63 (m, 2H), 7.58-7.56 (m, 1H), 7.43-7.35 (m, 4H), 5.04-4.98 (m, 1H), 4.01-3.89 (m, 2H), 3.76 (dd, J=13.5, 3.6 Hz, 0.4H), 3.56 (dd, J=12.6, 7.6 Hz, 0.6H), 3.46-3.41 (m, 1H), 3.40 (s, 3H), 3.25 (s, 3H), 3.22-3.02 (m, 4H), 2.87 (dd, J=14.1, 4.9 Hz, 0.5H), 2.73 (dd, J=14.1, 3.5 Hz, 0.5H), 2.47-2.30 (m, 2H).

### Example 4-Synthesis of (2S,6R)—N—((S)-1-cyano-2-(4-(3-methyl-2-oxo-2,3-dihydrobenzo[d] oxazol-5-yl)phenyl)ethyl)-6-methoxy-1,4-oxazepane-2-carboxamide (INSM-203)

Synthetic scheme for the preparation of INSM-203:

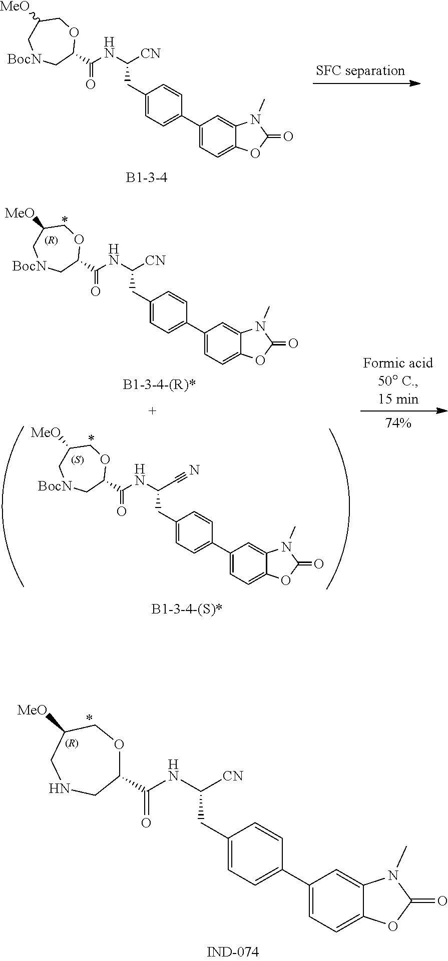

### Experimental Procedure

### tert-butyl (2S,6R*)-2-{[(1S)-1-cyano-2-[4-(3-methyl-2-oxo-2,3-dihydro-1,3-benzoxazol-5-yl)phenyl] ethyl]carbamoyl}-6-methoxy-1,4-oxazepane-4-carboxylate B1-3-4-(R)* & tert-butyl (2S,6S*)-2-{[(1S)-1-cyano-2-[4-(3-methyl-2-oxo-2,3-dihydro-1,3-benzoxazol-5-yl)phenyl] ethyl]carbamoyl}-6-methoxy-1,4-oxazepane-4-carboxylate B1-3-4-(S)*

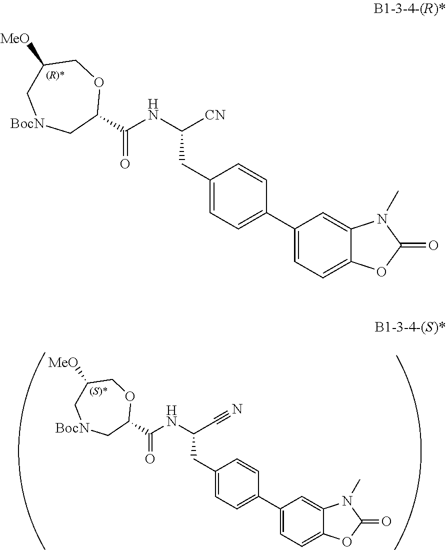

The diastereomeric mixture B1-3-4 (170 mg) was separated by preparative SFC (column: Chiralcel OD-H (30×250 mm); eluent: i-PrOH/CO₂, 30:70 isocratic conditions; Flow rate: 160 mL/min; T° C.: 35° C.) affording B1-3-4-(S)* (63.5 mg, 37%) & B1-3-4-(R)* (79 mg, 46%) as yellow solids. The stereochemistry(S)* was arbitrarily assigned to the first eluted product by preparative SFC and then the second eluted product was assigned (R)*.

B1-3-4-(S)*: Chiral SFC purity analysis: Rt=1.49 min, 100%, [M+Na]⁺=573.2. LC/MS (AN01_001_012): Rt=2.48 min, 100%, [M-C₄H₈+H]⁺=495.2.

B1-3-4-(R)*: Chiral SFC purity analysis: Rt=2.27 min, 100%, [M+Na]⁺=573.2. LC/MS (AN01_001_012): Rt=2.47 min, 100%, [M-C₄H₈+H]⁺=495.2.

### (2S,6R*)—N-[(1S)-1-cyano-2-[4-(3-methyl-2-oxo-2,3-dihydro-1,3-benzoxazol-5-yl)phenyl] ethyl]-6-methoxy-1,4-oxazepane-2-carboxamide INSM-203

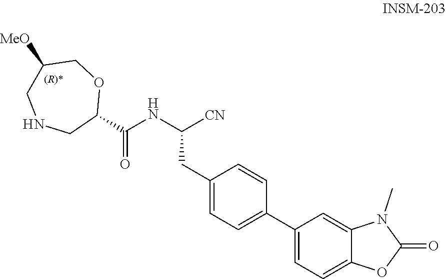 [SMILES: CO[C@@H]1CNC[C@@H](C(=O)N[C@H](C#N)Cc2ccc(-c3ccc4oc(=O)n(C)c4c3)cc2)OC1]

Starting from B1-3-4-(R)* (1 eq., 70.0 mg, 0.127 mmol) using general procedure C, INSM-202 was obtained as a white solid (42.5 mg, 74%) after purification by silica gel flash chromatography (4 g, gradient: DCM/MeOH from 100:0 to 93:7).

LC/MS (AN01_001_026): Rt=6.56 min, 99.9%, [M+H]⁺=451.5.

¹H NMR (DMSO-d₆, 400 MHz): δ ppm 8.63 (d, J=8.5 Hz, 1H), 7.66 (d, J=8.2 Hz, 2H), 7.58-7.55 (m, 1H), 7.43-7.37 (m, 4H), 5.01 (q, J=8.5 Hz, 1H), 4.01-3.96 (m, 2H), 3.56 (dd, J=12.5, 7.0 Hz, 1H), 3.46-3.41 (m, 1H), 3.40 (s, 3H), 3.25 (s, 3H), 3.22-3.14 (m, 2H), 3.04 (dd, J=14.0, 3.5 Hz, 1H), 2.87 (dd, J=14.2, 4.9 Hz, 1H), 2.73 (dd, J=14.2, 3.6 Hz, 1H), 2.43 (dd, J=14.2, 8.4 Hz, 1H), 2.35-2.22 (m, 1H).

### Example 3-DPP1 IC50 Measurements

The following compounds were tested:

| Compound | Chemical Structure |
|---|---|
| INSM-201 | 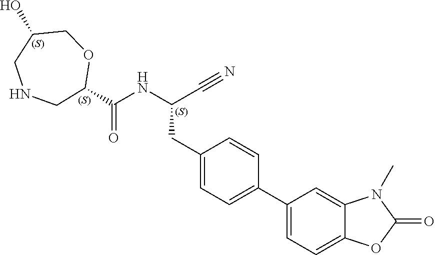 |
| INSM-202 | 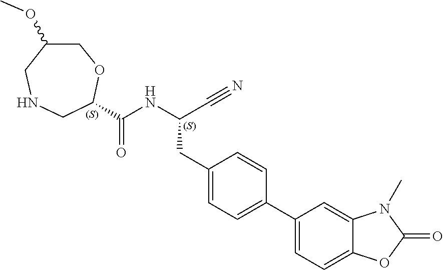 |
| INSM-203 | 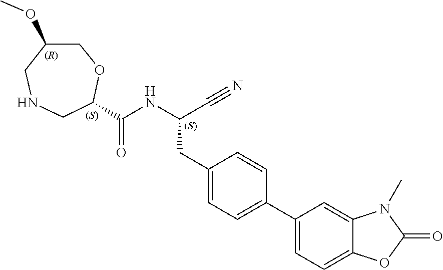 |
| INSM-204 | 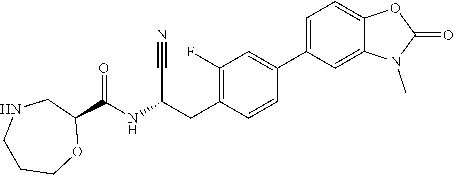 [SMILES: Cn1c(=O)oc2ccc(-c3ccc(C[C@@H](C#N)NC(=O)[C@@H]4CNCCCO4)c(F)c3)cc21] |

### Human DPP1 Enzyme IC50 Assay

Recombinant human DPP1 enzyme (R&D Systems; Minneapolis, MN) was first proteolytically processed into its mature form using recombinant human cathepsin L (R&D Systems) in a buffer consisting of 20 mM citric acid pH 4.5, 150 mM NaCl, 1 mM EDTA and 10 mM DTT. Alternatively, a crude lysate of HL-60 cells (ATCC; Manassas, VA) is used as a source of human DPP1 enzyme in the assay. Lysate was prepared in 1% Triton X-100 in PBS at a concentration of 20,000 live cells per μL of lysis buffer and centrifuged at 16,000×g for 10 minutes at 4° C., after which supernatant was collected and flash-frozen in liquid nitrogen.

Test articles were applied to activated human DPP1 enzyme in Assay Buffer (25 mM MES pH 6.0, 50 mM NaCl, 5 mM DTT) in a total reaction volume of 125 μL. 25 μL of compound in Assay Buffer plus 5% DMSO was first added to 50 μL of activated human DPP1 enzyme at a concentration of 1 ng/μL and allowed to pre-incubate for 10 minutes at 37° C. after which 50 μL of 1000 μM H-Gly-Arg-AMC substrate (Bachem; St. Torrance, CA) was added, giving final substrate concentration of 400 μM and a final DMSO concentration of 1%. Substrate cleavage was measured for 90 minutes at 37° C., with fluorescence at Excitation/Emission 350/450 nm measured every 5 minutes. DPP1 concentration was interpolated based on its activity relative to a standard curve of activated human recombinant DPP1 enzyme. IC50 values for each compound were calculated via the XLFit (IDBS Version 5.3.1.3) Add-On to Microsoft Excel using the four parameter fit equation y=(A+ ((B−A)/(1+ ((C/x)\hat{ }D)))), which appears as equation number 205 (4 Parameter Logistic Model or Sigmoidal Dose-Response Model) in XLFit. Default constraints were used for each Parameter. IC50 was defined as the compound concentration at which 50% of enzyme activity was inhibited when compared to the no-compound control.

### Mouse DPP1 Enzyme IC50 Assay

Test articles were applied to active mouse DPP1 enzyme (R&D Systems; Minneapolis, MN) in Assay Buffer (50 mM MES pH 5.5, 50 mM NaCl, 5 mM DTT) in a total reaction volume of 125 μL. 25 μL of compound in Assay Buffer plus 5% DMSO was first added to 50 μL of active mouse DPP1 enzyme at a concentration of 62.5 μg/μL and allowed to pre-incubate for 10 minutes at 37° C. after which 50 μL of 1000 μM H-Gly-Arg-AMC substrate (Bachem; St. Torrance, CA) was added, giving final substrate concentration of 400 μM and a final DMSO concentration of 1%. Substrate cleavage was measured for 90 minutes at 37° C., with fluorescence at Excitation/Emission 350/450 nm measured every 5 minutes. DPP1 concentration was interpolated based on its activity relative to a standard curve of recombinant active mouse DPP1 enzyme. IC50 values for each compound were calculated via the XLFit (IDBS Version 5.3.1.3) Add-On to Microsoft Excel using the four parameter fit equation y= (A+ ((B−A)/(1+ ((C/x)\hat{ }D)))), which appears as equation number 205 (4 Parameter Logistic Model or Sigmoidal Dose-Response Model) in XLFit. Default constraints were used for each Parameter. IC50 was defined as the compound concentration at which 50% of enzyme activity was inhibited when compared to the no-compound control.

### DPP1 Cell IC50 Assay

HL-60 cells (ATCC; Manassas, VA) were maintained in RPMI-1640 supplemented with 20% heat-inactivated FBS and 1X Antibiotic Antimycotic (Cytiva; Marlborough, MA). Media was changed every three to four days and cells were not allowed to exceed 1×10⁶ cells per mL. Prior to assay, cells were collected by centrifugation at 500 rcf for 3 minutes, resuspended in RPMI and counted. Cells were diluted in RPMI to a concentration of 5×105 live cells per mL and transferred to black 96-well plates for assay, 60 μL per well. Test articles were diluted in RPMI plus 0.5% DMSO, and 20 μL was added to each assay well. Compound was allowed to pre-incubate with cells with gentle shaking at 100 rpm for 60 minutes at 37° C. in a cell culture incubator maintained at 5% CO₂, after which 20 μL of 500 μM H-Gly-Phe-AFC substrate (MP Biomedicals; Solon, OH) in RPMI was added to each well. Plates were returned to the incubator with shaking at 100 rpm for 30 minutes, after which fluorescence was measured at Excitation/Emission 400/505 nm. Percent (%) Inhibition was calculated from RFU values compared to control cell wells that received only RPMI plus 0.5% DMSO. IC50 values for each compound were calculated via the XLFit (IDBS Version 5.3.1.3) Add-On to Microsoft Excel using the four parameter fit equation y=(A+ ((B−A)/(1+ ((C/x)\hat{ }D)))), which appears as equation number 205 (4 Parameter Logistic Model or Sigmoidal Dose-Response Model) in XLFit. IC50 was defined as the compound concentration at which 50% of enzyme activity was inhibited when compared to the no-compound control.

### Results

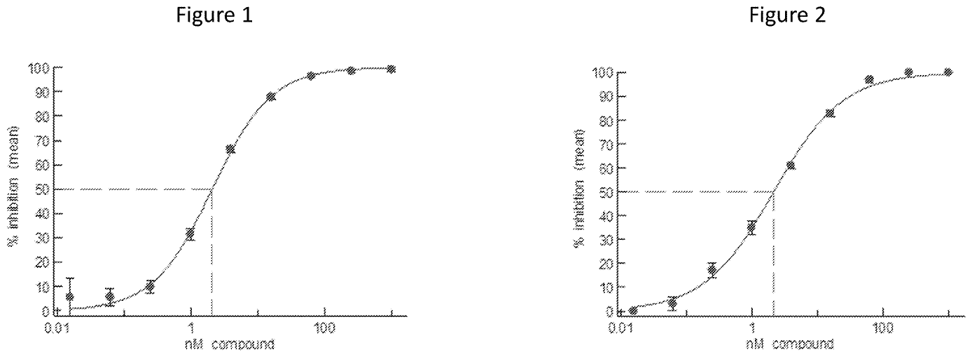

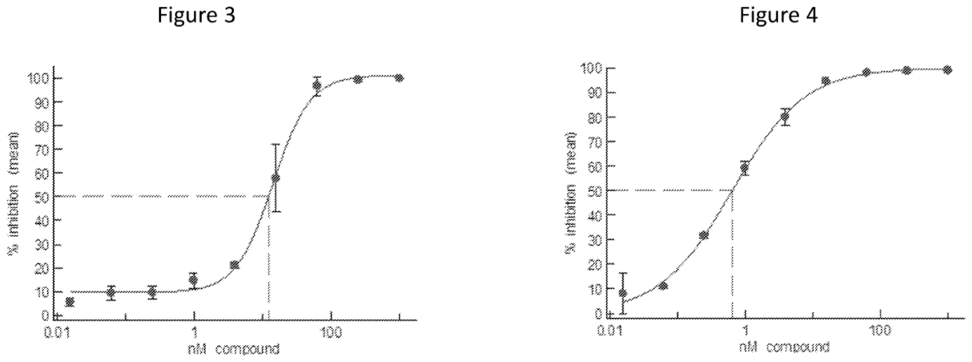

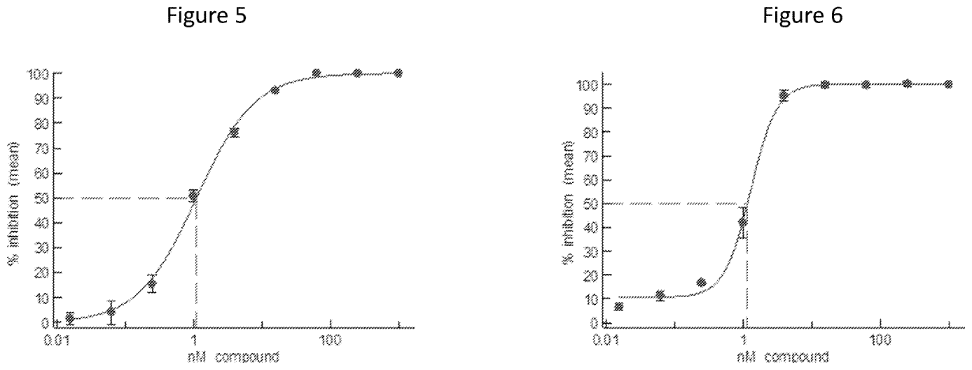

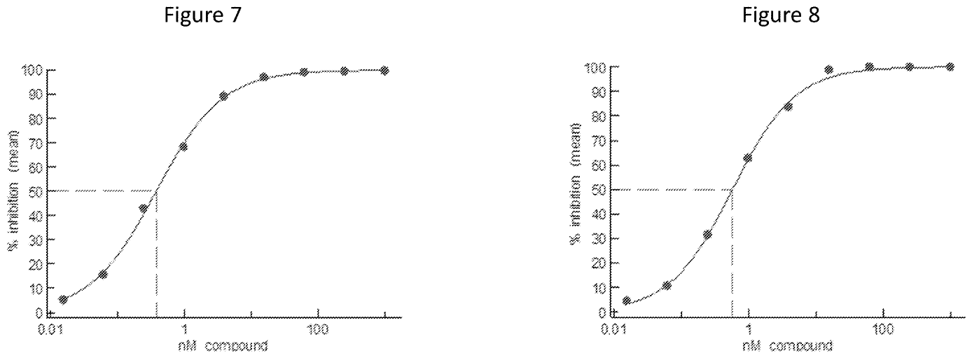

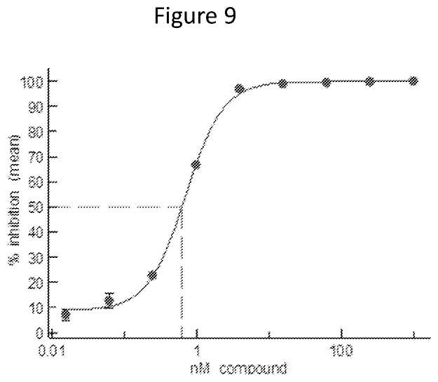

IC50 values for INSM-201, INSM-202, INSM-203 and INSM-204 are provided in Table 1. IC50 curves are also provided in FIGS. 1-9 for INSM-201 (FIGS. 1-3), INSM-202 (FIGS. 4-6), INSM-203 (FIGS. 7-9).

TABLE 1

| Com- | Mouse Enzyme | Human Enzyme | Cell assay |
|---|---|---|---|
| pound | IC50 (nM) | IC50 (nM) | IC50 (nM) |
| INSM-201 | 2.3 ± 0.4 (n = 3) | 2.03 ± 0.2 (n = 3) | 13.4 ± 4.1 (n = 3) |
| INSM-202 | 0.7 ± 0.2 (n = 3) | 1.03 ± 0.1 (n = 3) | 1.1 ± 0.2 (n = 3) |
| INSM-203 | 0.3 ± 0.1 (n = 3) | 0.6 ± 0.1 (n = 3) | 0.6 ± 0.01 (n = 3) |
| INSM-204 | 5.6 ± 0.4 (n = 2) | 8.7 ± 0.02 (n = 2) | 1.3 ± 0.3 (n = 3) |

### Example 4-In Vivo Activity of Compounds of Formula (I)

### Bone Marrow Cell Pellet Collection:

Male or female C57BL/6 mice at the age of 8-14 weeks were used.

The two femurs and two tibias were removed out of a single mouse and gauze was used to remove the muscles and residue tissues. A sterile environment for surgical instruments and surgical sites is maintained. The bones were put into one 15-mL tube with cold sterile RPMI medium and placed on ice until further processing is performed.

A fresh 50-mL tube is placed on ice and a fresh 40/70 μm cell strainer (CLS431750, Sigma) is inserted into the top of the Falcon tube. Both ends of two femurs and two tibias are cut with bone cutters and flush bone marrow of all bones from one single mouse are placed into the cell strainer. 5-mL of ice-cold RPMI (RPMI medium containing 10 mM HEPES, pH 7.4) was used for each femur or tibia using a blunt needle (25 G needle), totaling about 20 mL per tube.

The cell suspension is centrifuged at 600×g for 5 min at 4° C. and the supernatant is carefully discarded.

### Bone Marrow Lineage Cell Depletion Protocol

The cell pellet is resuspended in 400 μL of ice-cold MACS buffer (PH7.2 PBS plus 0.5% BSA and 2 mM EDTA) for one adult mouse (optimized for 4 bones/mouse).

100 μL Direct Lineage Cell Depletion Cocktail (which is enough for 100 million cells depletion) is added. The components are mixed well and incubated for 10 min. at 2° C.-8° C.

Magnetic separation LS Column (MACS, Miltenyi, 130 042 401) is prepared by placing it in the magnetic field of MACS Separator and rinsing with 3 mL of ice-cold MACS buffer.

Cell suspension is applied onto the column. Flow-through containing unlabeled cells, representing the enriched lineage negative cells, is collected.

Column is washed with 3×3 mL of ice-cold MACS buffer. Unlabeled cells that pass through are collected, representing the enriched lineage negative cells, and combined with the flow-through from prior step.

The cell suspension is centrifuged at 600×g for 5 min at 4° C., and the supernatant is carefully discarded. The pellet is washed once with 10 mL ice cold IMDM medium (IMDM GlutaMax (Gibco, 31980030) plus 10% FCS and 1% PS).

The cell pellet is resuspended with 2 mL ice-cold complete IMDM medium and the cells are counted. The cells are then ready for neutrophil differentiation.

Treatment of Bone Marrow Progenitor Cells with DPP1 Inhibitors and Differentiation into Neutrophils

The resulting undifferentiated bone marrow cells are in complete IMDM medium (IMDM with 10% FBS and 1% antibiotics); and the concentration of cells is adjusted to 1×10⁵ cells/mL, supplemented with 50 ng/ml of SCF (BioLegend, 579704) and 50 ng/ml of IL 3 (BioLegend, 575504). The cells are subgrouped and the respective DPP1 inhibitors are added accordingly. Cells are then cultivated in a flask at 37° C. for 3 days.

Cells are checked each day for expansion and maintaining a final concentration between 2-10×10⁵ cells/ml. On day 3, cells are counted. 3-4 million cells for each group are harvested and lystates prepared for NSPs activity detection. The left cells are resuspended at 2× 10⁵ cells/mL in fresh medium supplemented with 50 ng/ml of SCF, 50 ng/ml of IL 3 and 50 ng/ml of G-CSF (BioLegend, 574604). The DPP1 inhibitors are refreshed in the medium. Cells are then cultivated in a flask at 37° C. for another 2 days.

On day 5, cells are counted. 3-4 million cells/condition are harvested and the cells are lysed to prepare samples for NSPs activity detection. The remaining cells are collected by centrifugation at 600×g for 5 min. The left cells are washed once in 1×PBS. After the wash, the pelleted cells are resuspended in complete IMDM medium at 4-6×10⁵ cells/mL supplemented with 50 ng/ml of G-CSF only. The DPP1 inhibitors in the medium are refreshed. Cells incubate for additional 2 days, for a total of 7 days from day of harvest. On day 7, cells are counted. 3-4 million cells/condition are harvested and the cell lysed to prepare samples for NSPs activity detection.

### Store Cell Lysate for NSPs Activity Evaluation Protocol

3-4 million cells are collected into Eppendorf tubes. Samples are spun down at 600×g, 4° C. for 5 min. The supernatant is removed and resuspended in 1 mL PBS. The samples are spun down at 600×g, 4° C. for 5 min, and the supernatant is removed. The samples are lysed with 100 μL of ice-cold 1% (v/v) triton X-100, PBS and incubated on ice for 15 min. The samples are spun down at 16,000×g, 4° C. for 10 minutes. The lysate is collected and stored at −80° C. after snap freezing with liquid nitrogen.

### Enzyme Activities

Neutrophil elastase (NE), proteinase 3 (PR3), and cathepsin G (CatG) enzyme activities were determined in mouse progenitor cell lysates via kinetic assays using the following peptide substrates (final concentrations are indicated): for NE, 100 μM N-methoxysuccinyl-Ala-Ala-Pro-Val-7-amido-4-methylcoumarin (Sigma, St. Louis, MO; excitation/emission at 350/450 nm); for PR3, 40 μM (7-methoxycoumarin-4-yl) acetyl-lysyl-(picolinoyl)-Tyr-Asp-Ala-Lys-Gly-Asp-N-3-(2-4-dinitrophenyl)-2-3-diaminopropyonyl-NH₂) (GenScript, Piscataway, NJ; excitation/emission at 340/430 nm); and for CatG, 200 μM N-succinyl-Ala-Ala-Pro-Phe p nitroanilide (Sigma; absorbance at 405 nm).

Fluorescence or absorbance was quantified using a Synergy microplate reader (BioTek; Winooski, VT). The specific NSP activity in each sample was calculated as total activity minus the activity measured in the presence of a specific NSP inhibitor-elastase inhibitor (Abcam) for NE, sivelestat (Abcam) for PR3, and cathepsin G inhibitor I (Cayman Chemical; Ann Arbor, MI) for CatG. Active NSP concentrations were interpolated based on their activities relative to the standard curves created using active human NE protein (Sigma), active human PR3 protein (Sigma), and active human CatG protein (Sigma), respectively. Due to the unavailability of commercial mouse NE, PR3, and CatG proteins, the corresponding human proteins were used since their catalytic properties are predominantly conserved across species. A portion of each cell lysate sample was also set aside for protein quantitation using a Pierce BCA Protein Assay Kit (Thermo Fisher). NSP activities were normalized for the cell lysate protein concentrations. Data analysis was performed by first determining the linear portion of the kinetic run using an internally developed macro-Excel program. Slope values were calculated from the linear portion of the run, and standard curves were created using the standard slope values and their respective known concentrations. The unknown sample concentrations were then calculated using the second-degree polynomial line of best fit formula from the appropriate standard curves.

Results of the enzyme assays are provided in Tables 2-4, below.

INSM-201 observed concentration-dependent reduction in NE activity, and the maximum inhibition observed was ~85% at 0.1 μM regardless of day. The low dose INSM-201 observed fluctuations in inhibition depending on the day (range: 42-78%). INSM-202 appeared to reach NE inhibition plateau by 0.02 μM, and the maximum inhibition observed was ~85%, regardless of day. INSM-203 appeared to reach inhibition plateau by 0.02 μM, and the max inhibition observed was ~85%, regardless of day.

Results of the PR3 activity assays are provided in Table 3. INSM-201 observed concentration-dependent reduction in PR3 activity, and the max inhibition observed was ~95% at 1 μM on D5 and D7. Low dose fluctuations in inhibition were observed for INSM-201, depending on the day (range: 36%-72%). INSM-202 appeared to reach an inhibition plateau by 0.0 μM, and the max inhibition observed was ~95%, regardless of day. INSM-203 appeared to reach inhibition plateau by 0.02 μM; and the max inhibition observed was ~90%, regardless of day.

Results of the CatG activity assays are provided in Table 4. INSM-201 observed concentration-dependent reduction in CatG activity, and the max inhibition observed was ~95% at 1 μM, regardless of day. Low dose INSM-201 observed fluctuations in inhibition depending on the day (range: 27-78%). INSM-202 appeared to reach inhibition plateau by 0.02 μM, and the max inhibition observed was ~95%, regardless of day. INSM-203 appeared to reach an inhibition plateau by 0.02 μM, and the max inhibition was observed to be ~95%, regardless of day.

TABLE 2
Neutrophil Elastase Activity (% reduction from control
in test article (TA) treated vs. control cells.

| | INSM- | INSM- | INSM- | INSM- | INSM- | INSM- |
|---|---|---|---|---|---|---|
| | 201 | 201 | 202 | 202 | 203 | 203 |
| | (0.02 | (0.1 | (0.02 | (0.1 | (0.02 | (0.1 |
| | μM) | μM) | μM) | μM) | μM) | μM) |
| Day 3 | 67% | 83% | 86% | 78% | 89% | 91% |
| Day 5 | 42% | 83% | 80% | 90% | 82% | 84% |
| Day 7 | 78% | 84% | 89% | 88% | 84% | 86% |

TABLE 3
PR3 Activity (% reduction from control in test
article (TA) treated vs. control cells.

| | INSM- | INSM- | INSM- | INSM- | INSM- | INSM- |
|---|---|---|---|---|---|---|
| | 201 | 201 | 202 | 202 | 203 | 203 |
| | (0.02 | (0.1 | (0.02 | (0.1 | (0.02 | (0.1 |
| | μM) | μM) | μM) | μM) | μM) | μM) |
| Day 3 | 45% | 79% | 94% | 94% | 93% | 94% |
| Day 5 | 36% | 96% | 95% | 96% | 93% | 94% |
| Day 7 | 72% | 96% | 93% | 98% | 85% | 84% |

TABLE 4
CatG Activity (% reduction from control in test
article (TA) treated vs. control cells.

| | INSM- | INSM- | INSM- | INSM- | INSM- | INSM- |
|---|---|---|---|---|---|---|
| | 201 | 201 | 202 | 202 | 203 | 203 |
| | (0.02 | (0.1 | (0.02 | (0.1 | (0.02 | (0.1 |
| | μM) | μM) | μM) | μM) | μM) | μM) |
| Day 3 | 46% | 90% | 93% | 100% | 93% | 99% |
| Day 5 | 27% | 94% | 94% | 100% | 97% | 100% |
| Day 7 | 78% | 98% | 99% | 100% | 94% | 96% |

All, documents, patents, patent applications, publications, product descriptions, and protocols which are cited throughout this application are incorporated herein by reference in their entireties for all purposes.

The embodiments illustrated and discussed in this specification are intended only to teach those skilled in the art the best way known to the inventors to make and use the invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

1. A method of treating a disorder mediated by dipeptidyl peptidase 1 (DPP1) in a subject in need of treatment, comprising administering a composition comprising an effective amount of a compound of Formula (I), or a pharmaceutically acceptable salt thereof, to the subject for an administration period: 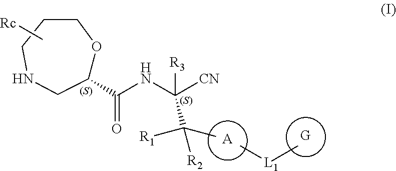
wherein,
G is a 5-12 membered carbon ring, a 5-12 membered monocyclic heterocycle containing 1-3 heteroatoms selected from N, S or O or the fused ring of formula 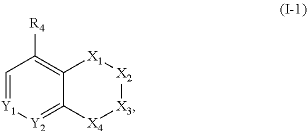
L₁ bonds G by replacing any hydrogen atom on ring G, and is a bond, C₁₋₃ alkylene, —NH—, —N(C₁₋₄alkyl)-, —O—, —S—, C₂₋₆ortho-alkenyl, C₂₋₆ortho-alkynyl, —CO— or —CONH—, wherein the alkylene, ortho-alkenyl or ortho-alkynyl is optionally substituted with 1-3 halogen, C₁₋₄ alkyl, cyano, hydroxyl, NH₂ and —COOH groups;
R₁, R₂ and R₃ are independently selected from H, deuterium, halogen, C₁₋₄alkyl, C₁₋₄alkoxy, C₂₋₆ alkenyl, C₂₋₆alkenyl, C₂₋₆alkynyl, C₃₋₆cycloalkyl and a 4-7 membered heterocycle containing 1-3 heteroatoms selected from N, S or O, the alkyl, alkoxy, alkenyl, alkynyl, cycloalkyl and heterocycle having arbitrarily undergone substitution with 1-3 groups selected from halogen, C₁₋₄alkyl, cyano, hydroxyl, NH₂ and COOH; alternatively, R₁ and R₂ form a C₃₋₆cycloalkyl or a 4-7 membered heterocycle containing 1-3 heteroatoms selected from N, S or O, wherein the cycloalkyl or heterocycle is optionally substituted with 1-3 groups selected from ═O, halogen, cyano, hydroxyl, NH₂, COOH, C₁₋₄ alkyl, C₂₋₆ alkenyl, C₂₋₆alkynyl and C₃₋₆cycloalkyl;
Y₁ and Y₂ are both independently selected from CR₄ or N;
each R₄ is independently selected from H, deuterium, halogen, C₁₋₄ alkyl, C₁₋₄ alkoxy, C₂₋₆ alkenyl, C₂₋₆ alkynyl, C₃₋₆cycloalkyl, cyano, hydroxyl, NH₂, NHC₁₋₄ alkyl, N(C₁₋₄ alkyl) 2, COOH, COC₁₋₄ alkyl, COOC₁₋₄ alkyl, CONHC₁₋₄ alkyl, CON(C₁₋₄ alkyl) 2, NHCOC₁₋₄ alkyl, and a 4-7 membered heterocycle containing 1-3 heteroatoms selected from N, S or O; wherein the alkyl, alkenyl, alkynyl, cycloalkyl and heterocycle groups within R₄ are optionally substituted with 1-3 groups selected from deuterium, halogen, cyano, hydroxyl, NH₂ and COOH;
X₁, X₂, X₃ and X₄ are each independently selected from a bond, NR₅, O, CR₆R₇, S, S(O) and S(O) 2, and wherein at most one of X₁, X₂, X₃ and X₄ is a bond;
Rc is H, —O, halogen, cyano, hydroxyl, NH₂, —COOH, C₁₋₄ alkyl, halogenated C₁₋₄ alkyl, hydroxyl C₁₋₄ alkyl, C₁₋₄ alkoxy, C₁₋₆ alkoxyalkyl or halogenated C₁₋₄ alkoxy;
each R₅ is independently selected from H, C₁₋₄ alkyl, —COC₁₋₄ alkyl, C₂₋₆ alkenyl, C₂₋₆alkynyl and C₃₋₆cycloalkyl; wherein the alkyl, alkenyl, alkynyl and cycloalkyl is optionally substituted with 1-₃ groups selected from deuterium, halogen, cyano, hydroxyl, NH₂ and COOH;
R₆ and R₇ are each independently selected from H, deuterium, halogen, C₁₋₄ alkyl, C₁₋₄alkoxy, C₂-₆ alkenyl, C₂₋₆ alkynyl, C₃₋₆cycloalkyl, cyano, hydroxyl, NH₂, COOH, and a 4-7 membered heterocycle containing 1-3 heteroatoms selected from N, S or O, wherein the alkyl, alkoxy, alkenyl, alkynyl, cycloalkyl and heterocycle is optionally substituted with 1-3 groups arbitrarily selected from deuterium, halogen, cyano, hydroxyl, NH₂ and —COOH;
alternatively, R₆ and R₇ form ═O;
alternatively, two R₅ on the atoms in adjacent rings or two R₆ on the atoms in adjacent rings within X₁, X₂, X₃ and X₄, or the R₅ and R₆ of atoms on adjacent rings and the atoms binding to them form double-bonds;
alternatively, the R₆ and R₇ on the same carbon atom form a C₃₋₁₂ carbon ring with the carbon atoms linked to them or a 4-7 membered heterocycle containing 1-3 heteroatoms selected from N, S or O, said carbon ring or heterocycle having arbitrarily undergone substitution with 1-3 groups selected from ═O, halogen, cyano, hydroxyl, NH₂, —COOH, C₁₋₄ alkyl, C₂₋₆ alkenyl, C₂₋₆ alkynyl and C₃₋₆ cycloalkyl;
Z is CH or N;
A is 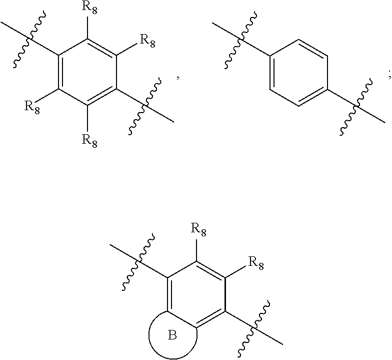
a hexatomic heteroaryl or 5-7 membered non-aromatic monocyclic heterocycle, wherein the heteroaryl and non-aromatic monocyclic heterocycle contains 1-3 heteroatoms selected from N, S or O;
B is a C₄₋₆ carbon ring or a 5-6 membered heterocycle containing 1-3 heteroatoms selected from N, S or O; and
each R₈ is independently selected from H, ═O, deuterium, halogen, C₁₋₄ alkyl, C₁₋₄ alkoxy, SC₁₋₄ alkyl, C₂₋₆ alkenyl, C₂₋₆ alkynyl, cyano, hydroxyl, —COOH, NH₂ and C₃₋₆ cycloalkyl; said alkyl, alkoxy, alkenyl, alkynyl or cycloalkyl having arbitrarily undergone substitution with 1-3 groups selected from deuterium, halogen, cyano, hydroxyl, NH₂ and COOH.

2. The method of claim 1, wherein the compound of Formula (I) is a compound of Formula (1a), or a pharmaceutically acceptable salt thereof: 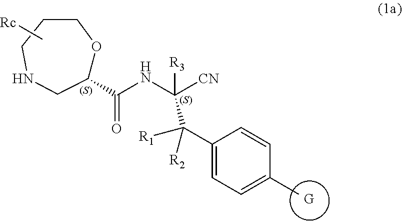
wherein
Rc is ═O, halogen, cyano, hydroxyl, NH₂, —COOH, C₁₋₄ alkyl, halogenated C₁₋₄ alkyl, hydroxyl C₁₋₄ alkyl, C₁₋₄ alkoxy, C₁₋₆ alkoxyalkyl or halogenated C₁₋₄ alkoxy.

3. The method of claim 1, wherein the compound of Formula (I) is a compound of Formula (1b), or a pharmaceutically acceptable salt thereof: 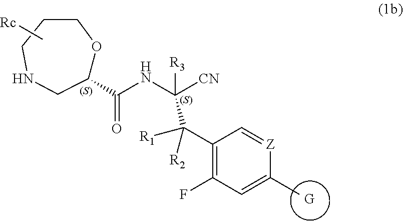
wherein
Rc is ═O, halogen, cyano, hydroxyl, NH₂, —COOH, C₁₋₄ alkyl, halogenated C₁₋₄ alkyl, hydroxyl C₁₋₄ alkyl, C₁₋₄ alkoxy, C₁₋₆ alkoxyalkyl or halogenated C₁₋₄ alkoxy.

4. The method of claim 1, wherein G is

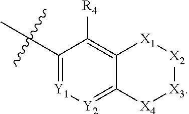

5. The method of claim 1, wherein G is

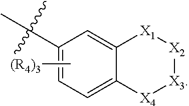

6. The method of claim 1, wherein G is

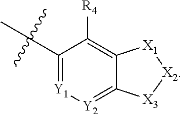

7. The method of claim 1, wherein G is

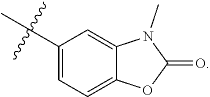 [SMILES: *c1ccc2oc(=O)n(C)c2c1]

8. The method of claim 1, wherein G is a substituted cyclopentane, cyclohexane, cycloheptane or benzene,

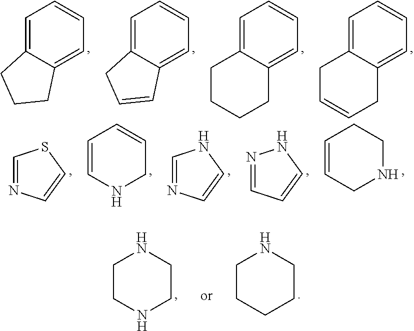

9. The method of claim 1, wherein G is:

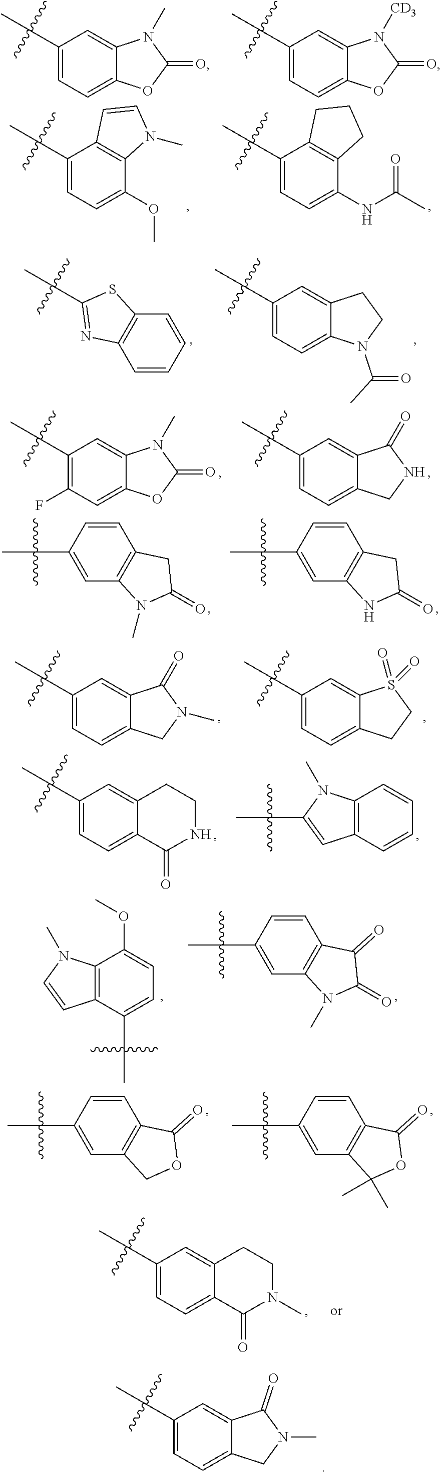

10. The method of claim 1, wherein G is

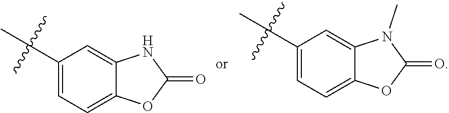 [SMILES: *c1ccc2oc(=O)[nH]c2c1 ; *c1ccc2oc(=O)n(C)c2c1]

11. The method of claim 1, wherein G is

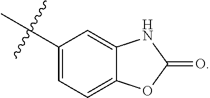 [SMILES: *c1ccc2oc(=O)[nH]c2c1]

12. The method of claim 1, wherein G is

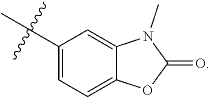 [SMILES: *c1ccc2oc(=O)n(C)c2c1]

13. The method of claim 1, wherein R₁ is H.

14. The method of claim 1, wherein R₂ is H.

15. The method of claim 1, wherein R₃ is H.

16. The method of claim 1, wherein Rc is hydroxyl, C₁₋₄ alkyl, or C₁₋₄ alkoxy.

17. (canceled)

18. The method of claim 1, wherein Rc is methoxy.

19.-36. (canceled)

37. The method of claim 1, wherein the compound of Formula (I) is one of the following compounds: 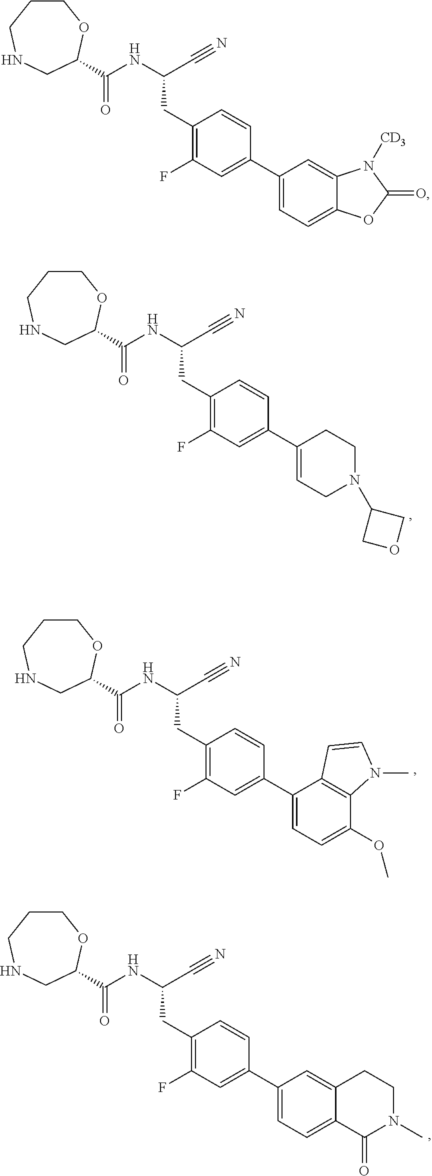 [SMILES: [2H]C([2H])([2H])n1c(=O)oc2ccc(-c3ccc(C[C@@H](C#N)NC(=O)[C@@H]4CNCCCO4)c(F)c3)cc21 ; N#C[C@H](Cc1ccc(C2=CCN(C3COC3)CC2)cc1F)NC(=O)[C@@H]1CNCCCO1 ; COc1ccc(-c2ccc(C[C@@H](C#N)NC(=O)[C@@H]3CNCCCO3)c(F)c2)c2ccn(C)c12 ; CN1CCc2cc(-c3ccc(C[C@@H](C#N)NC(=O)[C@@H]4CNCCCO4)c(F)c3)ccc2C1=O] 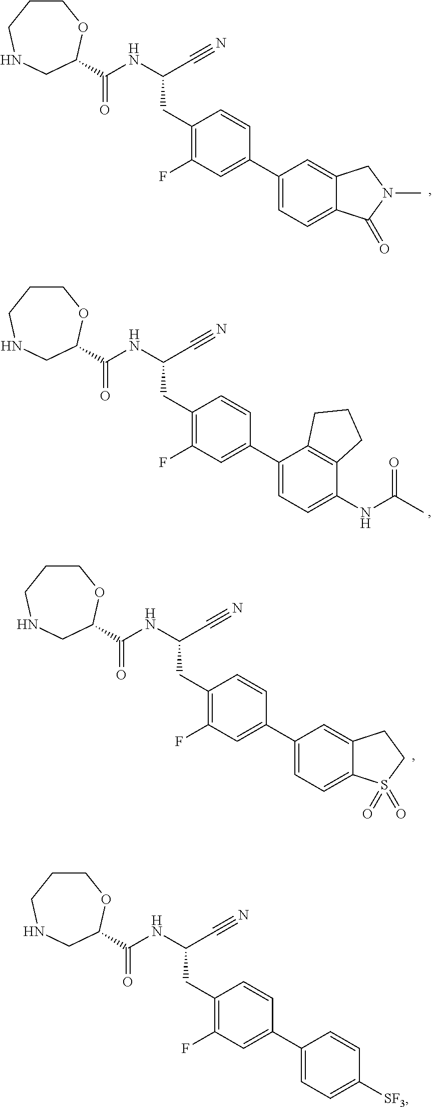 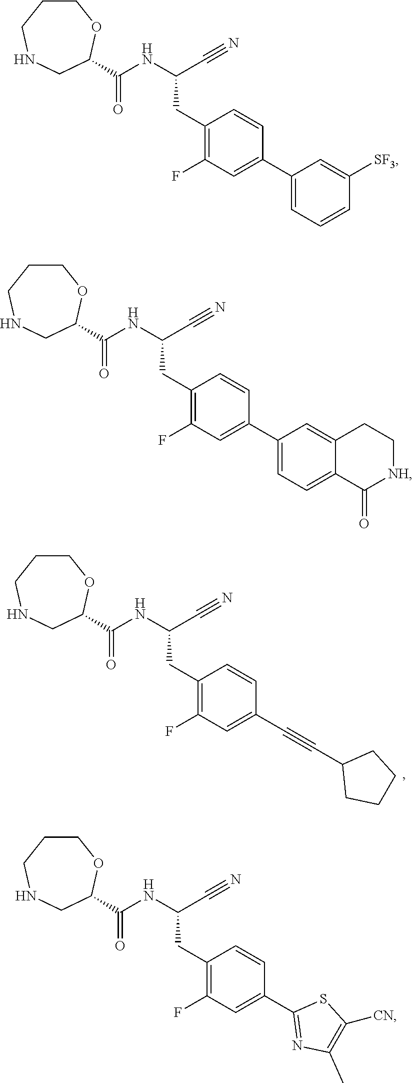 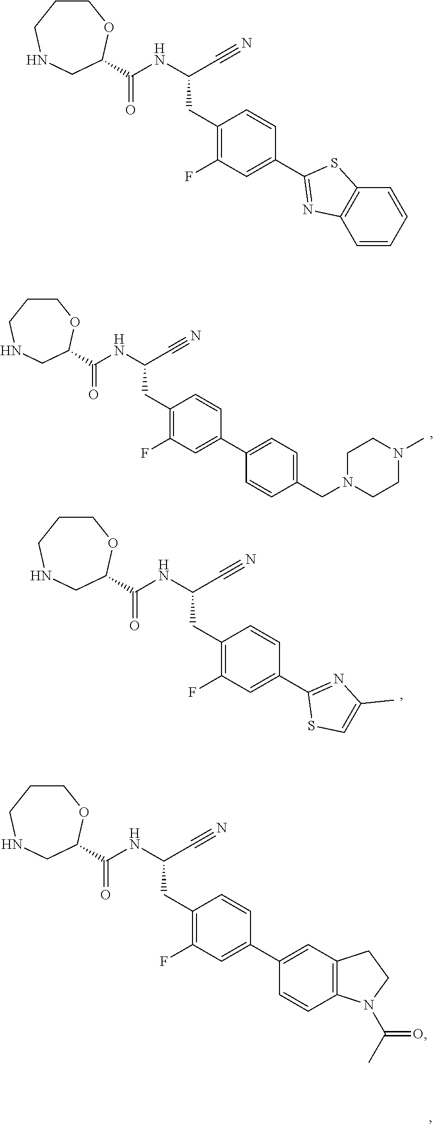 [SMILES: N#C[C@H](Cc1ccc(-c2nc3ccccc3s2)cc1F)NC(=O)[C@@H]1CNCCCO1 ; CN1CCN(Cc2ccc(-c3ccc(C[C@@H](C#N)NC(=O)[C@@H]4CNCCCO4)c(F)c3)cc2)CC1 ; Cc1csc(-c2ccc(C[C@@H](C#N)NC(=O)[C@@H]3CNCCCO3)c(F)c2)n1 ; CC(=O)N1CCc2cc(-c3ccc(C[C@@H](C#N)NC(=O)[C@@H]4CNCCCO4)c(F)c3)ccc21] 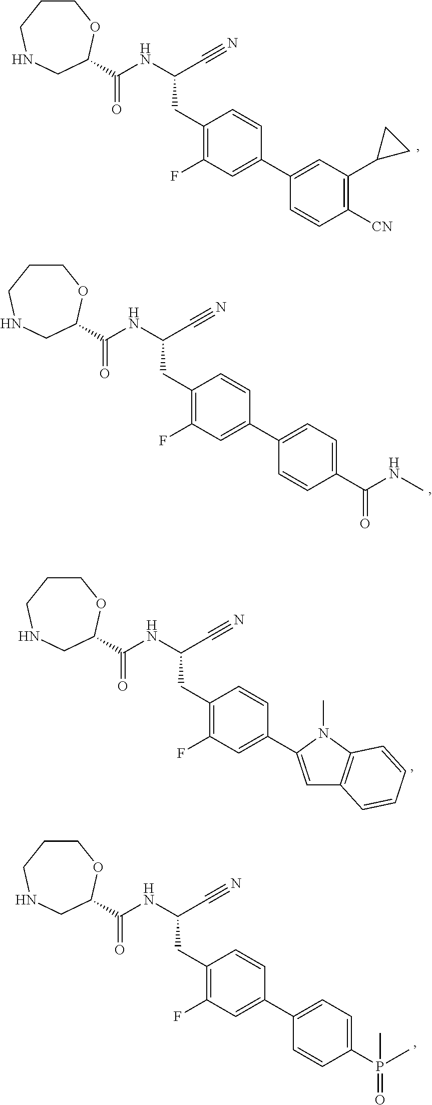 [SMILES: N#Cc1ccc(-c2ccc(C[C@@H](C#N)NC(=O)[C@@H]3CNCCCO3)c(F)c2)cc1C1CC1 ; CNC(=O)c1ccc(-c2ccc(C[C@@H](C#N)NC(=O)[C@@H]3CNCCCO3)c(F)c2)cc1 ; Cn1c(-c2ccc(C[C@@H](C#N)NC(=O)[C@@H]3CNCCCO3)c(F)c2)cc2ccccc21 ; CP(C)(=O)c1ccc(-c2ccc(C[C@@H](C#N)NC(=O)[C@@H]3CNCCCO3)c(F)c2)cc1] 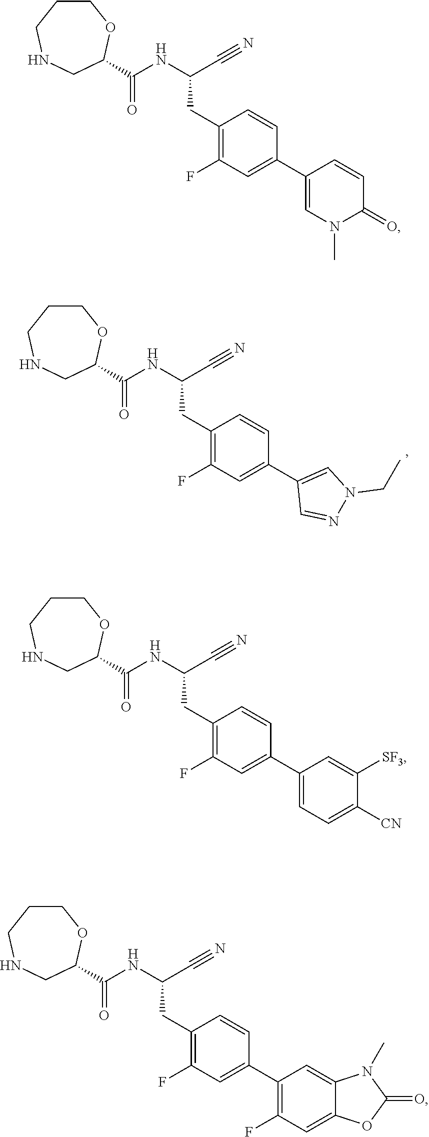 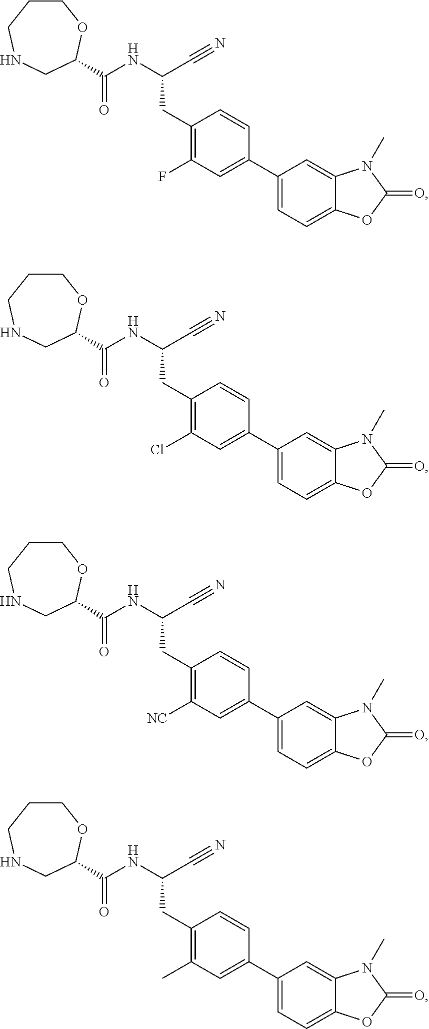 [SMILES: Cn1c(=O)oc2ccc(-c3ccc(C[C@@H](C#N)NC(=O)[C@@H]4CNCCCO4)c(F)c3)cc21 ; Cn1c(=O)oc2ccc(-c3ccc(C[C@@H](C#N)NC(=O)[C@@H]4CNCCCO4)c(Cl)c3)cc21 ; Cn1c(=O)oc2ccc(-c3ccc(C[C@@H](C#N)NC(=O)[C@@H]4CNCCCO4)c(C#N)c3)cc21 ; Cc1cc(-c2ccc3oc(=O)n(C)c3c2)ccc1C[C@@H](C#N)NC(=O)[C@@H]1CNCCCO1] 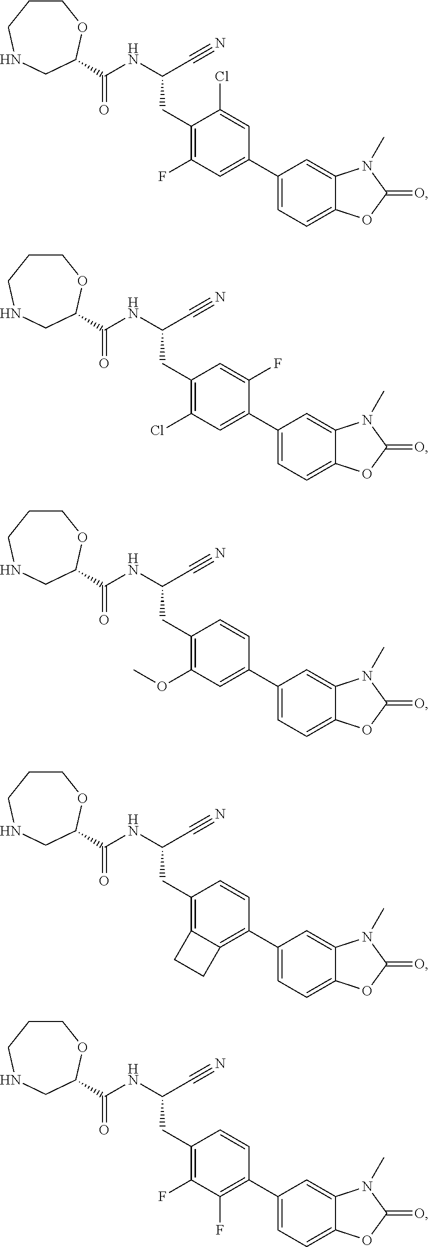 [SMILES: Cn1c(=O)oc2ccc(-c3cc(F)c(C[C@@H](C#N)NC(=O)[C@@H]4CNCCCO4)c(Cl)c3)cc21 ; Cn1c(=O)oc2ccc(-c3cc(Cl)c(C[C@@H](C#N)NC(=O)[C@@H]4CNCCCO4)cc3F)cc21 ; COc1cc(-c2ccc3oc(=O)n(C)c3c2)ccc1C[C@@H](C#N)NC(=O)[C@@H]1CNCCCO1 ; Cn1c(=O)oc2ccc(-c3ccc(C[C@@H](C#N)NC(=O)[C@@H]4CNCCCO4)c4c3CC4)cc21 ; Cn1c(=O)oc2ccc(-c3ccc(C[C@@H](C#N)NC(=O)[C@@H]4CNCCCO4)c(F)c3F)cc21] 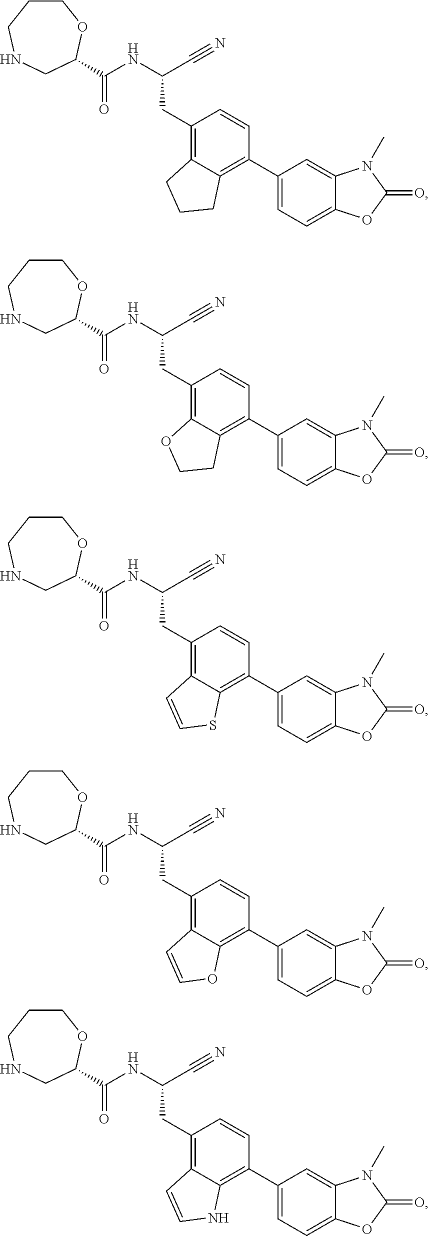 [SMILES: Cn1c(=O)oc2ccc(-c3ccc(C[C@@H](C#N)NC(=O)[C@@H]4CNCCCO4)c4c3CCC4)cc21 ; Cn1c(=O)oc2ccc(-c3ccc(C[C@@H](C#N)NC(=O)[C@@H]4CNCCCO4)c4c3CCO4)cc21 ; Cn1c(=O)oc2ccc(-c3ccc(C[C@@H](C#N)NC(=O)[C@@H]4CNCCCO4)c4ccsc34)cc21 ; Cn1c(=O)oc2ccc(-c3ccc(C[C@@H](C#N)NC(=O)[C@@H]4CNCCCO4)c4ccoc34)cc21 ; Cn1c(=O)oc2ccc(-c3ccc(C[C@@H](C#N)NC(=O)[C@@H]4CNCCCO4)c4cc[nH]c34)cc21] 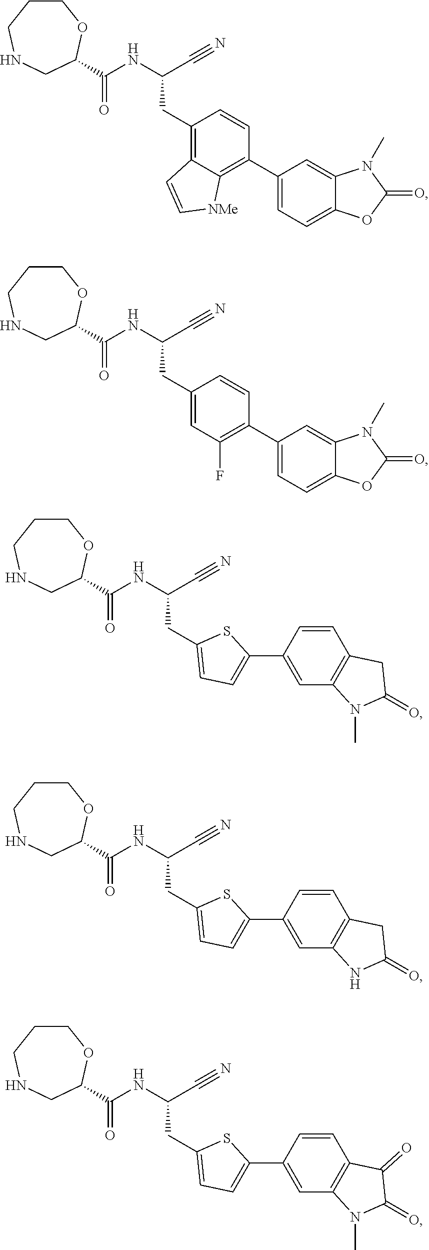 [SMILES: Cn1ccc2c(C[C@@H](C#N)NC(=O)[C@@H]3CNCCCO3)ccc(-c3ccc4oc(=O)n(C)c4c3)c21 ; Cn1c(=O)oc2ccc(-c3ccc(C[C@@H](C#N)NC(=O)[C@@H]4CNCCCO4)cc3F)cc21 ; CN1C(=O)Cc2ccc(-c3ccc(C[C@@H](C#N)NC(=O)[C@@H]4CNCCCO4)s3)cc21 ; N#C[C@H](Cc1ccc(-c2ccc3c(c2)NC(=O)C3)s1)NC(=O)[C@@H]1CNCCCO1 ; CN1C(=O)C(=O)c2ccc(-c3ccc(C[C@@H](C#N)NC(=O)[C@@H]4CNCCCO4)s3)cc21] 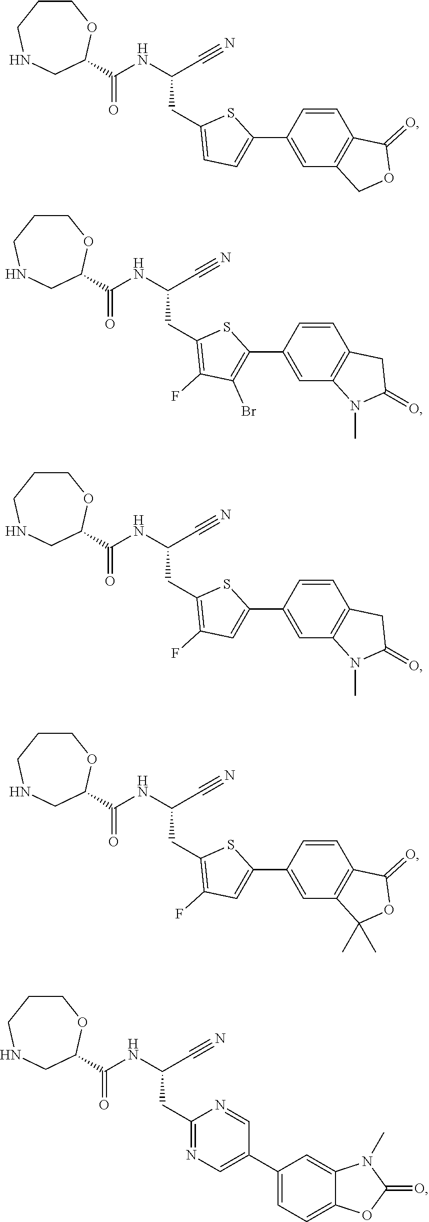 [SMILES: N#C[C@H](Cc1ccc(-c2ccc3c(c2)COC3=O)s1)NC(=O)[C@@H]1CNCCCO1 ; CN1C(=O)Cc2ccc(-c3sc(C[C@@H](C#N)NC(=O)[C@@H]4CNCCCO4)c(F)c3Br)cc21 ; CN1C(=O)Cc2ccc(-c3cc(F)c(C[C@@H](C#N)NC(=O)[C@@H]4CNCCCO4)s3)cc21 ; CC1(C)OC(=O)c2ccc(-c3cc(F)c(C[C@@H](C#N)NC(=O)[C@@H]4CNCCCO4)s3)cc21 ; Cn1c(=O)oc2ccc(-c3cnc(C[C@@H](C#N)NC(=O)[C@@H]4CNCCCO4)nc3)cc21] 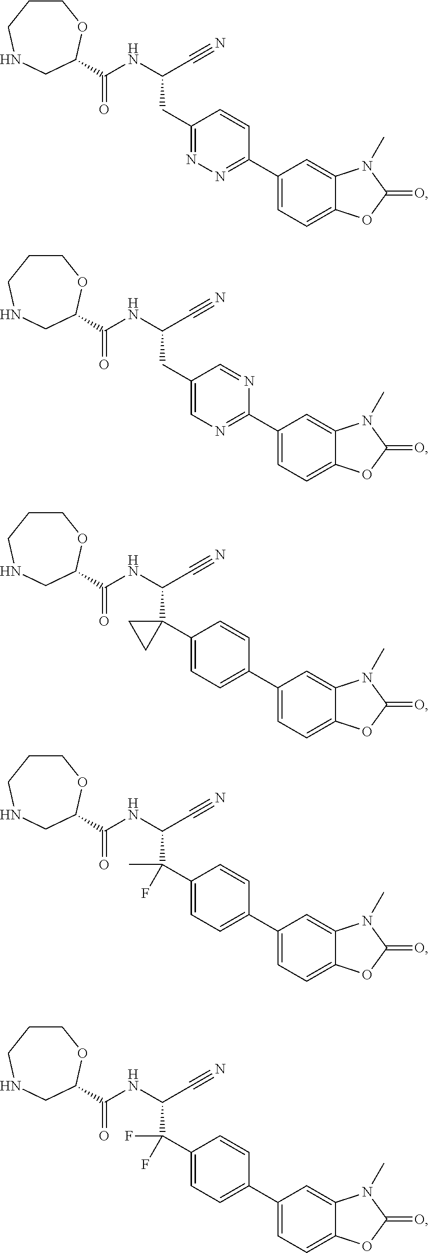 [SMILES: Cn1c(=O)oc2ccc(-c3ccc(C[C@@H](C#N)NC(=O)[C@@H]4CNCCCO4)nn3)cc21 ; Cn1c(=O)oc2ccc(-c3ncc(C[C@@H](C#N)NC(=O)[C@@H]4CNCCCO4)cn3)cc21 ; Cn1c(=O)oc2ccc(-c3ccc(C4([C@@H](C#N)NC(=O)[C@@H]5CNCCCO5)CC4)cc3)cc21 ; Cn1c(=O)oc2ccc(-c3ccc(C(C)(F)[C@@H](C#N)NC(=O)[C@@H]4CNCCCO4)cc3)cc21 ; Cn1c(=O)oc2ccc(-c3ccc(C(F)(F)[C@@H](C#N)NC(=O)[C@@H]4CNCCCO4)cc3)cc21] 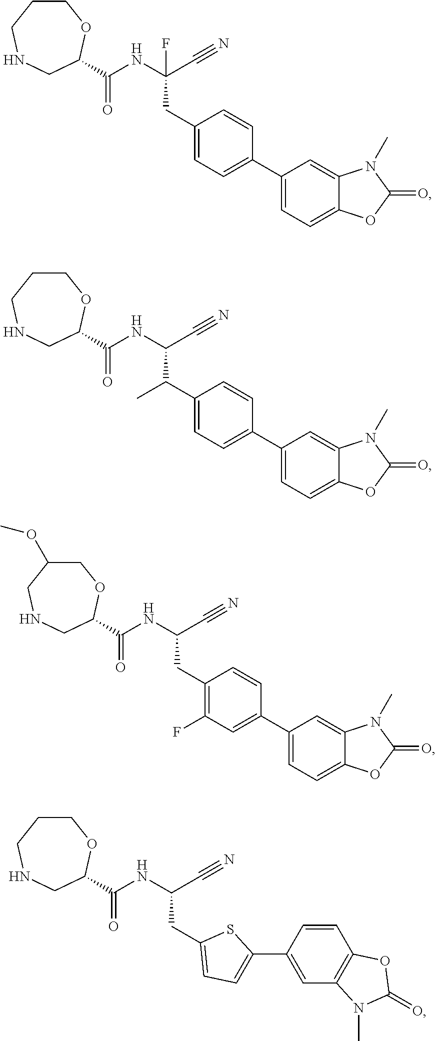 [SMILES: Cn1c(=O)oc2ccc(-c3ccc(C[C@](F)(C#N)NC(=O)[C@@H]4CNCCCO4)cc3)cc21 ; CC(c1ccc(-c2ccc3oc(=O)n(C)c3c2)cc1)[C@@H](C#N)NC(=O)[C@@H]1CNCCCO1 ; COC1CNC[C@@H](C(=O)N[C@H](C#N)Cc2ccc(-c3ccc4oc(=O)n(C)c4c3)cc2F)OC1 ; Cn1c(=O)oc2ccc(-c3ccc(C[C@@H](C#N)NC(=O)[C@@H]4CNCCCO4)s3)cc21] 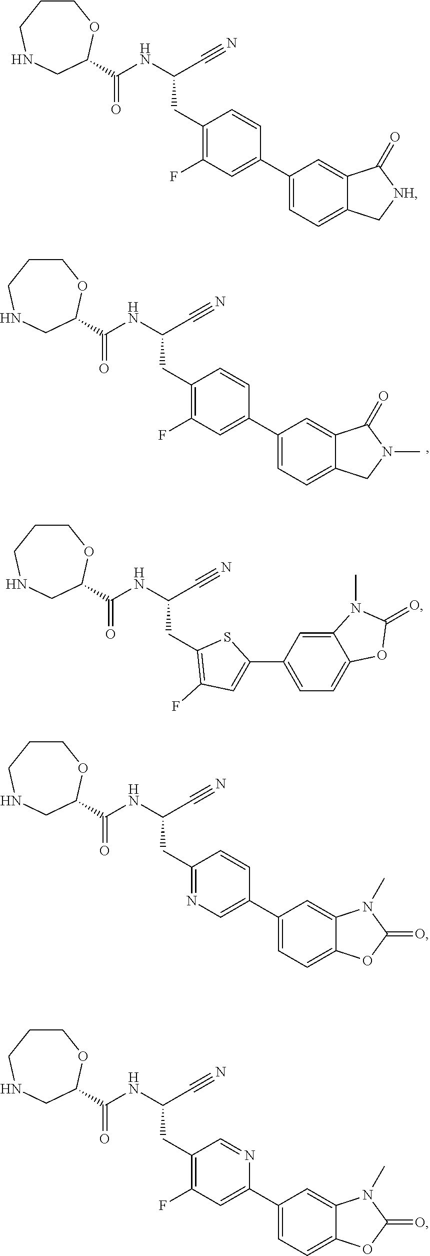 [SMILES: N#C[C@H](Cc1ccc(-c2ccc3c(c2)C(=O)NC3)cc1F)NC(=O)[C@@H]1CNCCCO1 ; CN1Cc2ccc(-c3ccc(C[C@@H](C#N)NC(=O)[C@@H]4CNCCCO4)c(F)c3)cc2C1=O ; Cn1c(=O)oc2ccc(-c3cc(F)c(C[C@@H](C#N)NC(=O)[C@@H]4CNCCCO4)s3)cc21 ; Cn1c(=O)oc2ccc(-c3ccc(C[C@@H](C#N)NC(=O)[C@@H]4CNCCCO4)nc3)cc21 ; Cn1c(=O)oc2ccc(-c3cc(F)c(C[C@@H](C#N)NC(=O)[C@@H]4CNCCCO4)cn3)cc21] 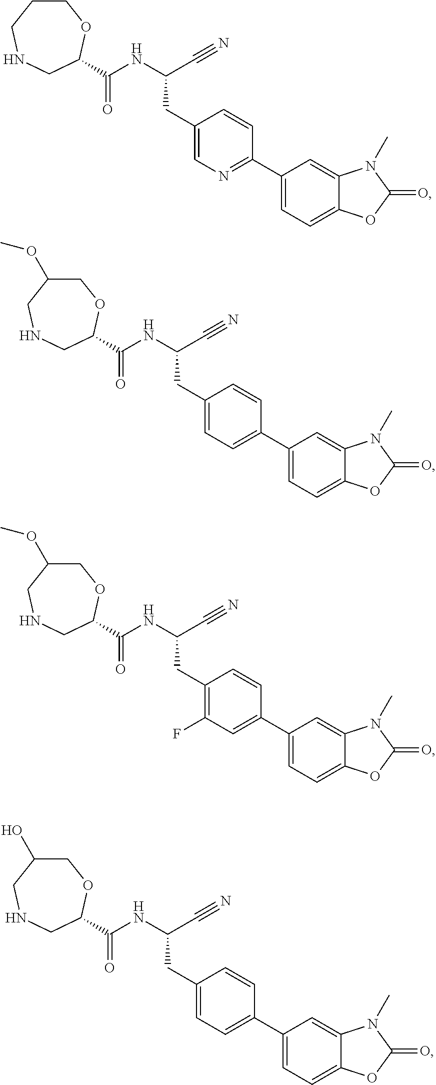 [SMILES: Cn1c(=O)oc2ccc(-c3ccc(C[C@@H](C#N)NC(=O)[C@@H]4CNCCCO4)cn3)cc21 ; COC1CNC[C@@H](C(=O)N[C@H](C#N)Cc2ccc(-c3ccc4oc(=O)n(C)c4c3)cc2)OC1 ; COC1CNC[C@@H](C(=O)N[C@H](C#N)Cc2ccc(-c3ccc4oc(=O)n(C)c4c3)cc2F)OC1 ; Cn1c(=O)oc2ccc(-c3ccc(C[C@@H](C#N)NC(=O)[C@@H]4CNCC(O)CO4)cc3)cc21] 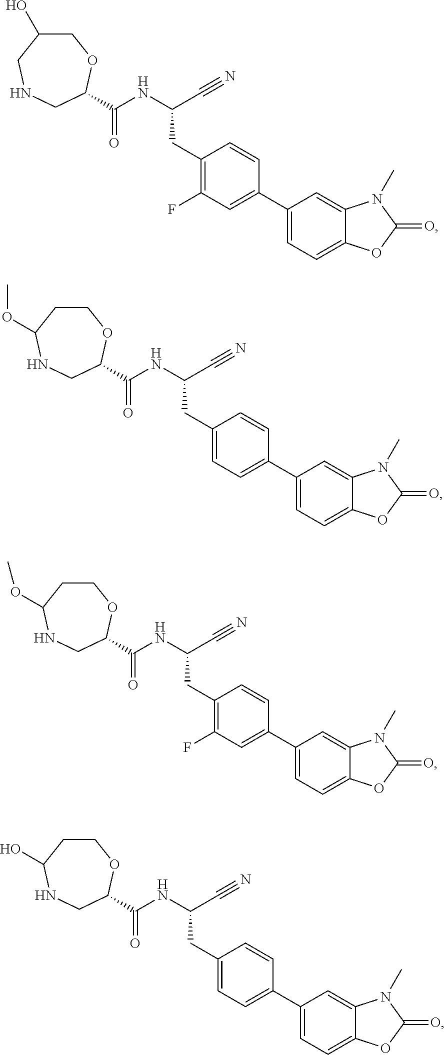 [SMILES: Cn1c(=O)oc2ccc(-c3ccc(C[C@@H](C#N)NC(=O)[C@@H]4CNCC(O)CO4)c(F)c3)cc21 ; COC1CCO[C@H](C(=O)N[C@H](C#N)Cc2ccc(-c3ccc4oc(=O)n(C)c4c3)cc2)CN1 ; COC1CCO[C@H](C(=O)N[C@H](C#N)Cc2ccc(-c3ccc4oc(=O)n(C)c4c3)cc2F)CN1 ; Cn1c(=O)oc2ccc(-c3ccc(C[C@@H](C#N)NC(=O)[C@@H]4CNC(O)CCO4)cc3)cc21] 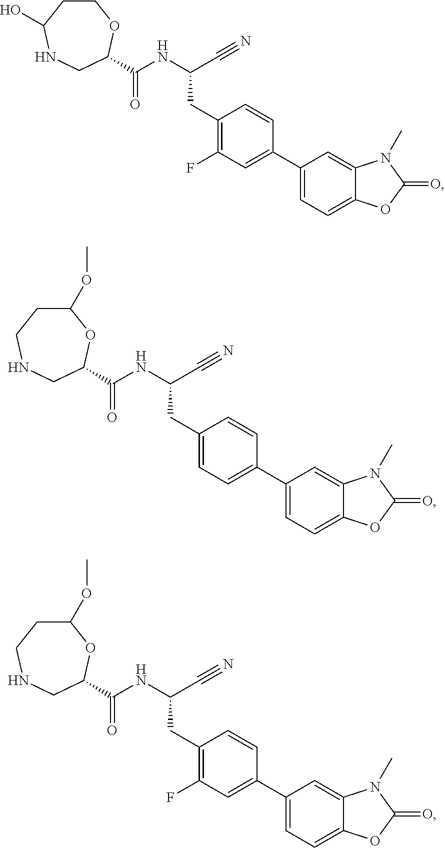 [SMILES: Cn1c(=O)oc2ccc(-c3ccc(C[C@@H](C#N)NC(=O)[C@@H]4CNC(O)CCO4)c(F)c3)cc21 ; COC1CCNC[C@@H](C(=O)N[C@H](C#N)Cc2ccc(-c3ccc4oc(=O)n(C)c4c3)cc2)O1 ; COC1CCNC[C@@H](C(=O)N[C@H](C#N)Cc2ccc(-c3ccc4oc(=O)n(C)c4c3)cc2F)O1] 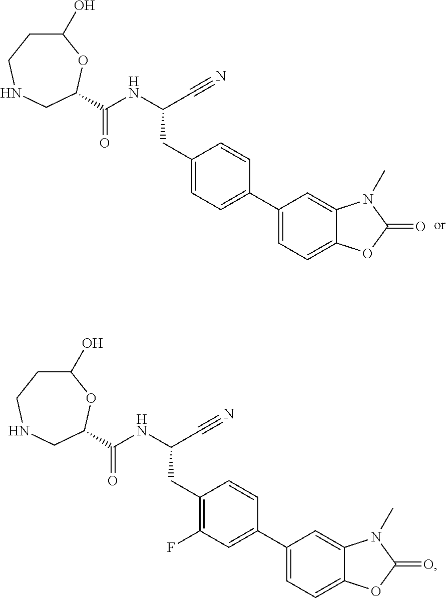 [SMILES: Cn1c(=O)oc2ccc(-c3ccc(C[C@@H](C#N)NC(=O)[C@@H]4CNCCC(O)O4)cc3)cc21 ; Cn1c(=O)oc2ccc(-c3ccc(C[C@@H](C#N)NC(=O)[C@@H]4CNCCC(O)O4)c(F)c3)cc21]
or
a pharmaceutically acceptable salt thereof.

38. The method of claim 1, wherein the compound of Formula (I) is 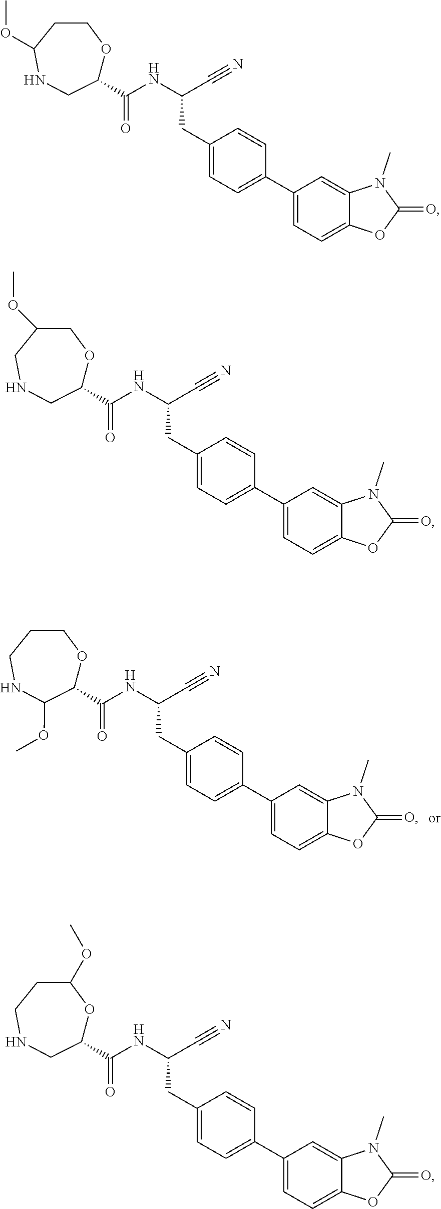 [SMILES: COC1CCO[C@H](C(=O)N[C@H](C#N)Cc2ccc(-c3ccc4oc(=O)n(C)c4c3)cc2)CN1 ; COC1CNC[C@@H](C(=O)N[C@H](C#N)Cc2ccc(-c3ccc4oc(=O)n(C)c4c3)cc2)OC1 ; COC1NCCCO[C@@H]1C(=O)N[C@H](C#N)Cc1ccc(-c2ccc3oc(=O)n(C)c3c2)cc1 ; COC1CCNC[C@@H](C(=O)N[C@H](C#N)Cc2ccc(-c3ccc4oc(=O)n(C)c4c3)cc2)O1]
or a pharmaceutically acceptable salt thereof.

39.-48. (canceled)

49. The method of claim 1, wherein the compound of Formula (I) is 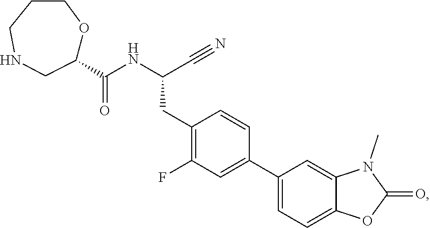 [SMILES: Cn1c(=O)oc2ccc(-c3ccc(C[C@@H](C#N)NC(=O)[C@@H]4CNCCCO4)c(F)c3)cc21]
or a pharmaceutically acceptable salt thereof.

50.-149. (canceled)
